(19) United States
(12) Patent Application Publication
(10) Pub. No.: US 2010/0196290 A1
(43) Pub. Date: Aug. 5, 2010

(54) COMPOSITIONS COMPRISING BETULONIC ACID

(75) Inventors: Jari Yli-Kauhaluoma, Helsinki (FI); Salme Koskimies, Helsinki (FI); Sami Alakurtti, Vantaa (FI); Pia Bergström, Kirkkonummi (FI); Thomas Ahlnäs, Kotka (FI); Kristian Meinander, Helsinki (FI); Päivi Tammela, Helsinki (FI)

Correspondence Address: BIRCH STEWART KOLASCH & BIRCH, PO BOX 747, FALLS CHURCH, VA, 22040-0747, US

(73) Assignee: Valtion teknillinen tutkimuskeskus, Espoo (FI)

(21) Appl. No.: 12/303,665

(22) PCT Filed: Jun. 6, 2007
(86) PCT No.: PCT/FI07/50333

§ 371 Date: Apr. 6, 2009

(30) Foreign Application Priority Data
Jun. 7, 2006 (FI) 20065388

(51) Int. Cl. A61K 8/63; A61Q 17/04; A61Q 1/06; C07J 53/00

(52) U.S. Cl. 424/59; 424/64; 552/510; 540/47

(57) ABSTRACT

The invention relates to compositions of cosmetic and pharmaceutical industries comprising betulonic acid for humans and animals, and further, to the use of betulonic acid in compositions of cosmetic and pharmaceutical industries. The invention is also directed to compositions containing, besides betulonic acid, optionally other compounds derived from betulin.

### FIELD OF THE INVENTION

The invention relates to compositions of cosmetic and pharmaceutical industries comprising betulonic acid for humans and animals, and further, to the use of betulonic acid in compositions of cosmetic and pharmaceutical industries. The invention is also directed to compositions containing besides betulonic acid optionally other compounds derived from betulin. Moreover, the invention relates to methods for the preparation of said compositions.

### PRIOR ART

Betulin having the structure 1 shown below is a naturally occurring pentacyclic triterpene alcohol of the lupane family, also known as betulinol and lup-20(29)-ene-3β,28-diol. Betulin is found in the bark of some tree species, particularly in the birch (Betula sp.) bark at best in amounts up to 40% of the bark dry weight. In addition to betulin, also minor amounts of betulin derivatives are obtained from tree bark. There are known methods mainly based on extraction for the isolation of betulin from bark material.

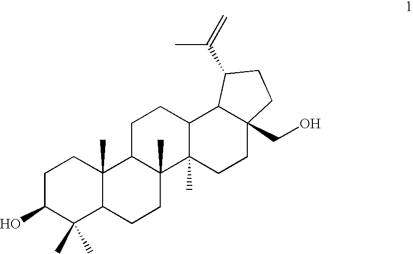

In some applications, poor solubility of betulin causes problems with respect to use and formulation, and accordingly, betulin is converted to its derivatives to improve the solubility. In the production of said derivatives, reactivities of the functional groups of betulin, that is, the primary and secondary hydroxyl groups and the double bond are typically utilized. Both hydroxyl groups may be esterified, thus obtaining mono- or diesters. Glycoside derivatives may be produced from betulin using known procedures, and betulin may be subjected to oxidation, reduction and rearrangement reactions in the presence of a suitable oxidation reagent, reducing reagent, or an acid catalyst, respectively.

Betulinic acid having the structure 3 shown in the reaction scheme below may be isolated e.g. from birch (Betula sp.) bark or cork of cork oak (Quercus suber L.) by extraction, and further, it may be produced by several methods mainly based on direct oxidation of the betulin or birch bark material. The reaction scheme shows the direct oxidation of betulin 1 according to U.S. Pat. No. 6,280,778 as Jones oxidation in the presence of a chromium(VI) oxide catalyst to give betulonic acid 2, followed by the selective reduction of the betulonic acid 2 thus obtained with sodium borohydride to give betulinic acid 3.

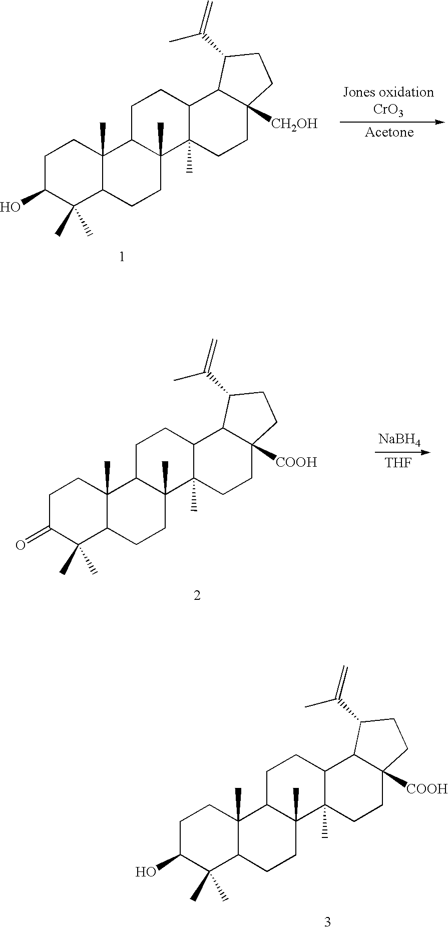

An alternative process for the production of betulinic acid is disclosed in U.S. Pat. No. 5,804,575, comprising an oxidation step where the 3-beta-hydroxyl of betulin is protected by acetylation. Isomerization and oxidation of the secondary hydroxyl group of betulin is thus prevented.

Suitability of betulin and the derivatives thereof for medical and cosmetic applications and for industrial chemicals is known to some extent, and further, antimicrobial activities of some of the compounds have also been studied.

Use of betulin and betulinic acid in cosmetic applications such as promoters of hair growth and thickness and as components in skin creams is already known for instance from WO 0003749. The document WO 0174327 discloses the use of betulinic acid in sun creams for the prevention of detrimental effects of the UV light.

Use of betulinic acid in pharmaceutical compositions or in cosmetic compositions for skin care, either as the sole active agent or in combination with ascorbic acid and conventional, pharmaceutically acceptable carriers is disclosed in EP 0 717 983. The betulinic acid in the composition stimulates collagen synthesis of the skin, the composition being suitable for care of wrinkled and flabby skin damaged by light and for treatment of cellulite. Betulinic acid may be a pure compound, or plant extract obtained from birch.

U.S. Pat. No. 6,207,711 discloses triterpenoid derivatives and salts thereof to be used for the prevention of aging due to light. In said derivatives, hydrogen at the position 28 of betulinic acid is replaced with the group —CHR₁R₂ where R₁ represents a phenyl, methoxyphenyl, ethoxyphenyl, butoxyphenyl, nitrophenyl, diphenyl or naphthyl group and R₂ represents a hydrogen atom or a phenyl group. Activities reducing wrinkles of the skin were found for said compounds in form of mixtures with additives conventionally used in skin formulations.

Compositions for the prevention and treatment of dry skin, aging and irritation of skin due to light, skin damages by UV radiation and for the improvement of self-tanning formulations are presented in WO 01/74327. Said compositions contain a protease inhibitor to prevent the decomposition of collagen and elastan by the protease enzyme, and a promoter of cellular differentiation. Suitable protease inhibitors include plant extracts containing triterpenoids such as extracts from birch, as well as betulin and betulinic acid compounds present in the extract. Suitable promoters of cellular differentiation include sclareolide, forskolin, 7-dehydrocholesterol, and vitamin D₃ analogs.

The document WO 2006/050158 discloses cosmetic preparations for skin and hair care containing additives and esters or ethers of betulin soluble in oil conventionally used in preparations for skin and hair care. Said preparations are endowed with properties protecting and treating skin and hair.

Use of betulin and some derivatives thereof as antifungal and anti-yeast agents is described in U.S. Pat. No. 6,642,217.

Antibacterial properties of betulin and some derivatives thereof are presented in WO 026762 (=US 2002/0119935). Said compounds are particularly active against the bacteria Escherichia coli, Staphylococcus aureus and Enterococcus faecalis.

WO 03/062260 discloses novel quaternary amine derivatives of betulin and antibacterial, antifungal and surfactant activities thereof.

In cosmetic and dermatological formulations or in products for animals, it is important to minimize the amount of cytotoxic substances to prevent problems or detrimental effects for the user caused by use of the product. Several antimicrobial, antifungal and anti-mold agents are already as such very cytotoxic. Particularly in case of preparations of cosmetic and pharmaceutical industries for external use, more concern is directed to behaviour and activity of conventional antimicrobial preserving agents either alone or as a combination with other constituents of the compositions at the site of application such as on skin. For instance, chemical reactions are potentially caused by UV light, resulting in decomposition of the compounds or reactions thereof with other constituents possibly yielding detrimental or even toxic compounds and free radicals, said radicals being very dangerous to the skin and further to the organism following penetration thereof through the skin and to the circulation system of the user. This may result in damages of the skin of the user, said damages potentially inducing melanoma, skin aging and irritation reactions.

Betulin and several betulin derivatives may be dissolved, emulsified and/or formulated in water only with difficulty, and poorly converted into stable and acceptable preparations for pharmaceutical and cosmetic industries.

Thus, there is an obvious need to provide novel cosmetic and pharmaceutical compositions for humans and animals to be used externally, particularly on skin and hair, enabling to avoid or substantially reduce potential problems of cytotoxicity and at the same time to improve other desired properties and performance of the products.

Compounds derived from betulin refer here to pentacyclic triterpenoids, particularly to betulinic acid and betulin derivatives and particularly to those derivatives comprising natural compounds and/or compounds with known low toxicity as substituents, and especially to alcohol, phenol and/or carboxylic acid and/or ester and/or amide and/or ether derivatives of betulin and/or derivatives having a partial heterocyclic structure and/or carbamate derivatives.

Antibacterial compounds refer here to compounds with activity against bacteria, viruses, yeasts, molds, and fungi.

The term microbe refers to bacteria, viruses, yeasts, fungi, and molds.

### OBJECTS OF THE INVENTION

An object of the invention is to provide a composition of cosmetic or pharmaceutical industry for humans and animals, comprising betulonic acid.

Another object of the invention is to provide a composition of cosmetic or pharmaceutical industry for humans and animals, comprising betulonic acid, to be used externally.

Still another object of the invention is to provide a composition of cosmetic or pharmaceutical industry for humans and animals, comprising betulonic acid, to be used externally, said composition also containing one or more compound(s) derived from betulin.

Further, an object of the invention is the use of betulonic acid in composition of cosmetic or pharmaceutical industry for humans and animals.

An object of the invention is also the use of betulonic acid in composition of cosmetic or pharmaceutical industry for humans and animals in combination with one or more compound(s) derived from betulin.

An object of the invention is also a method for the preparation of compositions of cosmetic or pharmaceutical industry for humans and animals, said compositions containing betulonic acid.

Still another object of the invention is to provide a sun protective product comprising betulonic acid and optionally one or more compound(s) derived from betulin.

Still another object of the invention is to provide a skin care product comprising betulonic acid and optionally one or more compound(s) derived from betulin.

Still another object of the invention is to provide a lip care product comprising betulonic acid and optionally one or more compound(s) derived from betulin.

Still another object of the invention is to provide a coloured cosmetic product comprising betulonic acid and optionally one or more compound(s) derived from betulin.

Characteristic features of the compositions, the use thereof, and the methods according to the invention are disclosed in the claims.

### GENERAL DESCRIPTION OF THE INVENTION

The present invention relates to compositions of cosmetic and pharmaceutical industries for humans and animals, containing betulonic acid, which compositions may in addition contain one or more compound(s) derived from betulin. Preferably, said compositions are to be used externally for instance on skin or hair.

In addition to betulonic acid, the compounds may also contain other compounds derived from betulin. Cytotoxicity of betulonic acid and other betulin derivatives is low, and further, said compounds penetrate the skin only poorly, they have antimicrobial activity, they prevent detrimental effects of the UV light, and are stable and environmentally acceptable. Thus, they are very suitable for preparations that will be used externally and exposed to solar UV radiation and other environmental stresses at the site of application.

The invention is also directed to compositions comprising besides betulonic acid novel betulin derivatives comprising natural compounds and/or known compounds with low toxicity as substituents such as to alcohol, phenol and/or carboxylic acid and/or ester and/or amide and/or ether derivatives of betulin and/or derivatives with heterocyclic structural moieties and/or carbamate derivatives, particularly to carboxylic acid and ester and amide derivatives of betulin and/or derivatives with partial heterocyclic structures and/or carbamate derivatives. Said betulin derivatives have improved solubilities and/or emulsifiabilities in solvents or media used in cosmetic and pharmaceutical industries, and may be readily formulated into stable preparations with desired acitivities.

### DETAILED DESCRIPTION OF THE INVENTION

In products of the cosmetic and pharmaceutical industry for humans and animals and particularly in preparations for external use, such as products for skin and hair, it is important to minimize the amount of cytotoxic substances while the desired activity is obtained. It is also important to minimize the amount of cytotoxic substances at the site of use once the product is applied for instance on skin. It has surprisingly been found that the compositions are provided with the desired properties particularly by using betulonic acid. The activity may also be modified by the addition of, besides betulonic acid, one or more compounds derived from betulin defined below.

Betulonic acid and compounds derived from betulin are endowed with low cytotoxicity and simultaneously with superior antimicrobial activity particularly against bacteria, as may be seen from the results described in the examples. Moreover, these compounds are endowed with considerable antioxidant and anti-viral activities as well as inhibitory activity with respect to the apoptose of melanoma cells. Said compounds effectively prevent detrimental effects of UV light. In addition, the skin is only poorly penetrated by said compounds, and thus no silicone compounds are needed in the preparations for the prevention of said penetration. The compounds are stable and environmentally acceptable.

A composition of the cosmetic and pharmaceutical industry for humans and/or animals comprises between 0.01 and 20 and preferably 0.1 and 10% by weight of betulonic acid. Moreover, the composition may optionally contain between 0.01 and 20 and preferably 0.1 and 10% by weight of one or more compound(s) derived from betulin selected from the following group.

According to the invention, useful compounds derived from betulin include the following betulin derivatives having the general formula I shown below, and salts thereof

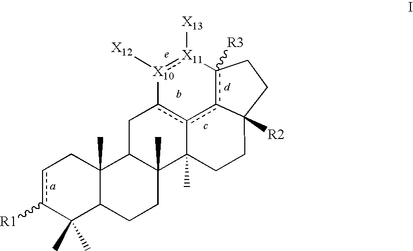

where R1=H, —OH, —ORₐ, —O(C═O)R_{b}, —NRₐR_{z}, —CN, —CHO, —(C═O)ORₐ, —SRₐ, —O(C═O)NHRₐ, ═O or ═S where Rₐ, R_{b} and R_{z}, independently represent H, C₁-C₂₂ aliphatic, unbranched or branched, saturated or unsaturated hydrocarbon residue, with the proviso that X₁₀═X₁₁ is not H; C₃-C₈ cyclic or heterocyclic residue; substituted or unsubstituted phenyl or benzyl residue; amine, amide or amino acid; substituted or unsubstituted 1,2,3-triazol, 1,2,4-triazol, tetrazol, pyrrole, isoxazol, pyrazol, imidazol, or oxazol; a carboxymethyl, carboxymethylester or carboxymethylamide derivative or a salt thereof;
R2=—CH₂ORₐ, —CH₂OH, —CH₂O(C═O)R_{b}, —(C═O)OR_{b}, —CH₂NRₐR_{z}, —CH₂CN, —CH₂CHO, —CH₂(C═O)ORₐ, —CH₂SRₐ, —CH₂O(C═O)NHRₐ, —CH═O or —CH═S where Rₐ, R_{b} and R_{z}, independently represent H, C₁-C₂₂ aliphatic, unbranched or branched, saturated or unsaturated hydrocarbon residue, with the proviso that X₁₀═X₁₁ is not H; C₃-C₈ cyclic or heterocyclic residue; substituted or unsubstituted phenyl or benzyl residue; amine, amide or amino acid; substituted or unsubstituted 1,2,3-triazol, 1,2,4-triazol, tetrazol, pyrrole, isoxazol, pyrazol, imidazol, or oxazol; a carboxymethyl, carboxymethylester or carboxymethylamide derivative or a salt thereof;
R3=isopropenyl, isopropyl, isopropylphenyl, isopropylhydroxyphenyl, or isopropylsuccinic acid derivative or a salt thereof;

### X₁₀═X₁₁═H, C Or N;

X₁₂═X₁₃=“absent”; (C═O)OR, (C═O)NHR where R═H or a C₁-C₆ linear or branched alkyl or alkenyl group or substituted or unsubstituted phenyl or benzyl residue or X₁₂—X₁₃ forms a cyclic partial structure of the form —(X₁₂═X₁₄)—X₁₅—(X₁₃═X₁₆)— where X₁₂═X₁₃═C, X₁₄═X₁₆=“absent”, O or S, X₁₅═C, O, S or N—X₁₇ where X₁₇═H, C₁-C₆ linear or branched alkyl or alkenyl group, substituted or unsubstituted phenyl or benzyl residue;
a, b, c and d independently represent a double or single bond; and
e=“absent” or represents a double or single bond.

In case X₁₀═X₁₁═H, X₁₂═X₁₃=“absent”, a, b, c and d each represent a single bond and e=“absent”, then R1, and Rₐ and R_{z} present in R2 independently represent a C₃-C₈ cyclic or heterocyclic residue, C₁-C₂₂ aliphatic, unbranched or branched, saturated or unsaturated hydrocarbon residue, with the proviso that at the same time R1 represents ═O (oxo) or ═S, a C₃-C₈ cyclic or heterocyclic residue, substituted or unsubstituted phenyl residue, substituted or unsubstituted 1,2,3-triazol, 1,2,4-triazol, tetrazol, pyrrole, isoxazol, pyrazol, imidazol, or oxazol, a carboxymethyl, carboxymethylester or carboxymethylamide derivative or a salt thereof and R_{b} represents a C₁₀-C₂₂ aliphatic, unbranched or branched, saturated or unsaturated hydrocarbon residue, C₃-C₈ cyclic or heterocyclic residue, substituted or unsubstituted phenyl or benzyl residue, substituted or unsubstituted 1,2,3-triazol, 1,2,4-triazol, tetrazol, pyrrole, isoxazol, pyrazol, imidazol, or oxazol, a carboxymethyl, carboxymethylester or carboxymethylamide derivative or a salt thereof.

According to the invention, preferable compounds derived from betulin include the compounds having the following structures IA-IQ:

### IA:

### R1=OH;

R2=CH₂O(C═O)R_{f} or —CH₂ORₐ(C═O)OR_{f} where R_{f}═C₃-C₈ cyclic or heterocyclic residue, substituted or unsubstituted phenyl or benzyl residue and Rₐ═C₁-C₂₂ linear or branched alkenyl or alkylene group;
R3=CH₂═CCH₃ (isopropenyl group);

### X₁₀═X₁₁═H;

X₁₂═X₁₃=“absent”;
a, b, c, and d each represent a single bond; and
e=“absent”.

### IB:

### R1=OH;

R2=CH₂O(C═O)(CHR_{g})CH₂COOY where R_{g}═C₄-C₂₂ linear or branched alkyl or alkenyl group, Y═H, Na, K, Ca, Mg, C₁-C₄-alkyl group, or NRₕ where Rₕ═H or C₁-C₄-alkyl group;

### R3=CH₂═CCH₃;

### X₁₀═X₁₁═H;

X₁₂═X₁₃=“absent”;
a, b, c, and d each represent a single bond; and
e=“absent”.

### IC:

### R1=OH;

R2=CH₂ORᵢ where Rᵢ=ornithine, N-acetylanthranilic acid or trimethylglycin ester (or betain ester);

### R3=CH₂═CCH₃;

### X₁₀═X₁₁═H;

X₁₂═X₁₃=“absent”;
a, b, c, and d each represent a single bond; and
e=“absent”.

### ID:

### R1=OH;

R2=CH₂O(C═O)CHRⱼ(NHZ) or —CH₂ORₐ(C═O)NHRⱼ where Rₐ═C₁-C₂₂ linear or branched alkylene or alkenyl group; Rⱼ═H, C₁-C₄-alkyl, benzyl, 4-hydroxybenzyl, —CH₂CH₂CH₂CH₂NH₂, 4-imidazolylmethyl or 3-indolylmethyl group, and Z═H, Rₖ, (C═O)Rₖ or COORₖ where Rₖ═C₁-C₂₂ branched or unbranched alkyl or alkenyl group, or a phenyl, benzyl or 4-hydroxybenzyl group;

### R3=CH₂═CCH₃;

### X₁₀═X₁₁═H;

X₁₂═X₁₃=“absent”;
a, b, c, and d each represent a single bond; and
e=“absent”.

### IE:

### R1=OH;

R2=CH₂ORₐ where Rₐ=an ester of carboxymethoxy substituted verbenol, terpineol, thymol, carvacrol, menthol, cinnamic alcohol, curcumin, eugenol, borneol, isoborneol, longifolol, isolongifolol, globulol, epiglobulol, sedrol, or episedrol, or an ester of chrysanthemic acid, cinnamic acid, or retinolic acid;

### R3=CH₂═CCH₃;

### X₁₀═X₁₁═H;

X₁₂═X₁₃=“absent”;
a, b, c, and d each represent a single bond; and
e=“absent”.

### IFa:

R1=O(C═O)Rₘ or —ORₐ(C═O)ORₘ, where Rₘ═C₃-C₈ cyclic or heterocyclic residue, substituted or unsubstituted phenyl residue, Rₐ═C₁-C₂₂ linear or branched alkylene or alkenyl group;
R2=CH₂O(C═O)Rₒ or —CH₂ORₐ(C═O)Rₒ where Rₒ═C₃-C₈ cyclic or heterocyclic residue, substituted or unsubstituted phenyl residue, Rₐ═C₁-C₂₂ linear or branched alkylene or alkenyl group;

### R3=CH₂═CCH₃;

### X₁₀═X₁₁H;

X₁₂═X₁₃=“absent”;
a, b, c, and d each represent a single bond; and
e=“absent”.

### IFb:

R1=O(C═O)(CHR_{c})CH₂COOY where R_{c}═C₄-C₂₂ linear or branched alkyl or alkenyl group, Y═H, Na, K, Ca, Mg, C₁-C₄ alkyl group or NRₕ, where Rₕ═H or a C₁-C₄ alkyl group;
R2=CH₂O(C═O)(CHR_{d})CH₂COOY where R_{d}═C₄-C₂₂ linear or branched alkyl or alkenyl group, Y═H, Na, K, Ca, Mg, C₁-C₄ alkyl group or NRₖ where Rₖ═H or a C₁-C₄ alkyl group;

### R3=CH₂═CCH₃;

### X₁₀═X₁₁═H;

X₁₂═X₁₃=“absent”;
a, b, c, and d each represent a single bond; and
e=“absent”.

### IFc:

R1=ORᵣ where Rᵣ=an ornithine ester, an ester of N-acetylanthranilic acid, or a trimethylglycine ester;
R2=CH₂ORₚ where Rₚ=an ornithine ester, an ester of N-acetylanthranilic acid, or a trimethylglycine ester;

### R3=CH₂═CCH₃;

### X₁₀═X₁₁═H;

X₁₂═X₁₃=“absent”;
a, b, c, and d each represent a single bond; and
e=“absent”.

### IFd:

R1=O(C═O)CHR₅(NHZ) or —ORₐ(C═O)NHR₅, where Rₐ═C₁-C₂₂ linear or branched alkenyl or alkylene group; Rₛ═H, C₁-C₄-alkyl, benzyl, 4-hydroxybenzyl, —CH₂CH₂CH₂CH₂NH₂, 4-imidazolylmethyl or 3-indolylmethyl group, Z═H, Rₖ, (C═O)Rₖ or COORₖ where Rₖ═C₁-C₂₂ branched or unbranched alkyl or alkenyl group, or a phenyl, benzyl or 4-hydroxybenzyl group;
R2=CH₂O(C═O)CHRₓ(NHZ) or —CH₂ORₐ(C═O)NHRₓ where Rₐ═C₁-C₂₂ linear or branched alkenyl or alkylene group; Rₓ═H, C₁-C₄-alkyl, benzyl, 4-hydroxybenzyl, —CH₂CH₂CH₂CH₂NH₂, 4-imidazolylmethyl or 3-indolylmethyl group, Z═H, R_{y}, (C═O)R_{y} or COOR_{y} where R_{y}═C₁-C₂₂ branched or unbranched alkyl or alkenyl group, or a phenyl, benzyl or 4-hydroxybenzyl group;

### R3=CH₂═CCH₃;

### X₁₀═X₁₁═H;

X₁₂═X₁₃=“absent”;
a, b, c, and d each represent a single bond; and
e=“absent”.

### IFe:

R1=OR where Rᵥ=an ester of carboxymethoxy substituted verbenol, terpineol, thymol, carvacrol, menthol, cinnamic alcohol, curcumin, eugenol, borneol, isoborneol, longifolol, isolongifolol, globulol, epiglobulol, sedrol, or episedrol, or an ester of chrysanthemic acid, cinnamic acid, or retinolic acid;
R2=CH₂OR where Rᵤ=an ester of carboxymethoxy substituted verbenol, terpineol, thymol, carvacrol, menthol, cinnamic alcohol, curcumin, eugenol, borneol, isoborneol, longifolol, isolongifolol, globulol, epiglobulol, sedrol, or episedrol, or an ester of chrysanthemic acid, cinnamic acid, or retinolic acid; and

### R3=CH₂═CCH₃;

### X₁₀═X₁₁═H;

X₁₂═X₁₃=“absent”;
a, b, c, and d each represent a single bond; and
e=“absent”.

### IG:

### R1=OH;

R2=(C═O)NHCHRₓCOOY where Y═H, Na, K, Ca, Mg, C₁-C₄ alkyl group or NR_{y} where R_{y}═H or a C₁-Cₐ alkyl group, and Rₓ═H, C₁-C₄-alkyl, benzyl, 4-hydroxybenzyl, —CH₂CH₂CH₂CH₂NH₂, 4-imidazolylmethyl or 3-indolylmethyl group or L-aspartate, L-histidine, L-glutamine or L-lysine;

### R3=CH₂═CCH₃;

### X₁₀═X₁₁═H;

X₁₂═X₁₃=“absent”;
a, b, c, and d each represent a single bond; and
e=“absent”.

### IH:

### R1=OH;

R2=(C═O)R_{w}, where R_{w}=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol;

### R3=CH₂═CCH₃;

### X₁₀═X₁₁H;

X₁₂═X₁₃=“absent”;
a, b, c, and d each represent a single bond; and
e=“absent”.

### IIa:

R1=OR where R═H, C₁-C₄ alkyl, benzyl, 4-hydroxybenzyl, —CH₂CH₂CH₂CH₂NH₂, 4-imidazolylmethyl, 3-indolylmethyl, or CH₃SCH₂ group, or an ester of carboxymethoxy substituted verbenol, terpineol, thymol, carvacrol, menthol, cinnamic alcohol, curcumin, eugenol, borneol, isoborneol, longifolol, isolongifolol, globulol, epiglobulol, sedrol, or episedrol, or an ester of chrysanthemic acid, cinnamic acid, or retinolic acid;
R2=(C═O)NHCHRₓCOOY where Y═H, Na, K, Ca, Mg, C₁-C₄ alkyl group or NR_{y} where R_{y}═H or a C₁-C₄ alkyl group, and Rₓ═H, C₁-C₄-alkyl, benzyl, 4-hydroxybenzyl, —CH₂CH₂CH₂CH₂NH₂, 4-imidazolylmethyl or 3-indolylmethyl group or L-aspartate, L-histidine, L-glutamine or L-lysine;

### R3=CH₂═CCH₃;

### X₁₀═X₁₁═H;

X₁₂═X₁₃=“absent”;
a, b, c, and d each represent a single bond; and
e=“absent”.

### IIb:

R1=OR where R═H, C₁-C₄ alkyl, benzyl, 4-hydroxybenzyl, —CH₂CH₂CH₂CH₂NH₂, or CH₃SCH₂ group, or an ester of carboxymethoxy substituted verbenol, terpineol, thymol, carvacrol, menthol, cinnamic alcohol, curcumin, eugenol, borneol, isoborneol, longifolol, isolongifolol, globulol, epiglobulol, sedrol, or episedrol, or an ester of chrysanthemic acid, cinnamic acid, or retinolic acid;
R2=(C═O)R_{w} where R_{w}=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol;

### R3=CH₂═CCH₃;

### X₁₀═X₁₁═H;

X₁₂═X₁₃=“absent”;
a, b, c, and d each represent a single bond; and
e=“absent”.

### IJa:

R1=oxo(═O) group;
R2=(C═O)NHCHRₓCOOY where Y═H, Na, K, Ca, Mg, C₁-C₄ alkyl group or NR_{y} where R_{y}═H or a C₁-C₄ alkyl group, and Rₓ═H, C₁-C₄-alkyl, benzyl, 4-hydroxybenzyl, —CH₂CH₂CH₂CH₂NH₂, 4-imidazolylmethyl or 3-indolylmethyl group or 28-aspartate dimethylester;

### R3=CH₂═CCH₃;

### X₁₀═X₁₁H;

X₁₂═X₁₃=“absent”;
a, b, c, and d each represent a single bond; and
e=“absent”.

### IJb:

R1=oxo(═O) group;
R2=(C═O)R_{w} where R_{w}=an ester of verbenol, terpineol, thymol, carvacrol, menthol, cinnamic alcohol, curcumin, eugenol, borneol, isoborneol, longifolol, isolongifolol, globulol, epiglobulol, sedrol, or episedrol;

### R3=CH₂═CCH₃ or CH₃—CH—CH₃;

### X₁₀═X₁₁═H;

X₁₂═X₁₃=“absent”;
a, b, c, and d each represent a single bond; and
e=“absent”.

### 1K:

R1=OH or O—(C═O)R_{b} where R_{b}═C₃-C₈ cyclic or heterocyclic residue, C₁-C₂₂ alkyl or alkenyl group or a phenyl group;
R2=CH₂OH or CH₂O—(C═O)R_{f} where R_{f}═C₃-C₈ cyclic or heterocyclic residue, C₁-C₂₂ alkyl or alkenyl group or a phenyl group;
R₃=(CH₃)₂CR_{z}, or CH₃CHCH₂R_{z} where R_{z}═C₆H₅₋ₙ(OH)ₙ or C₆H₅₋ₙ₋ₘ(OH)ₙ(OCH₃)ₘ and n=0-5, m=0-5, n+m≦5;

### X₁₀═X₁₁═H;

X₁₂═X₁₃=“absent”;
a, b, c, and d each represent a single bond; and
e=“absent”.

### IL:

R1=OH or O—(C═O)R_{b} where R_{b}═C₃-C₈ cyclic or heterocyclic residue, C₁-C₂₂ alkyl or alkenyl group or a phenyl group;
R2=CH₂OH or CH₂O—(C═O)R_{f} where R_{f}═C₃-C₈ cyclic or heterocyclic residue, C₁-C₂₂ alkyl or alkenyl group or a phenyl group; and
R₃═H₂C═CCH₂R_{q} or CH₃CCH₂R_{q} where R_{q}=succinic anhydride, succinic imide or CH(COOR₀CH₂COOR_{z}, where Rₒ═H, Na, K, Ca, Mg or a C₁-C₂₂ linear or branched alkyl or alkenyl group and R_{z}═H, Na, K, Ca, Mg or a C₁-C₂₂ linear or branched alkyl or alkenyl group;

### X₁₀═X₁₁H;

X₁₂═X₁₃=“absent”;
a, b, c, and d each represent a single bond; and
e=“absent”.

### IM:

R1=H, OR_{z}, O(C═O)R_{b}, NRₐR_{z}, CN, ═NORₐ, CHO, (C═O)OR_{z}, SR_{z}, ═O, ═S, where R_{z}═H, C₁-C₆ linear or branched alkyl or alkenyl group, or an aromatic group ZZ shown below, and Rₐ═H, C₁-C₆ linear or branched alkyl or alkenyl group, or an aromatic group ZZ, and R_{b}═H, C₁-C₂₂ linear or branched alkyl or alkenyl group, or an aromatic group ZZ, or R1 corresponds to the partial structure XX shown below;
R2=CH₂OR_{z}, CH₂O(C═O)R_{b}, (C═O)OR_{b}, CH₂NRₐR_{z}, CH₂CN, CN, CH═NORₐ, CH₂CHO, CH₂(C═O)OR_{z}, CH₂SR_{z}, CH═O, CH═S where R_{z}═H, C₁-C₆ linear or branched alkyl or alkenyl group, or an aromatic group ZZ, and Rₐ═H, C₁-C₆ linear or branched alkyl or alkenyl group, or an aromatic group ZZ, and R_{b}═C₁-C₂₂ linear or branched alkyl or alkenyl group, or an aromatic group ZZ, or R2 corresponds to the partial structure YY shown below;
R3=CH₂═C—CH₃ or CH₃—CH—CH₃ (isopropyl group);

### X₁₀═X₁₁═H;

X₁₂═X₁₃=“absent”;
a, b, c, and d independently represent a single or a double bond; and
e=“absent”;
said partial structures XX and YY where YY═CH₂XX are selected from the group consisting of:

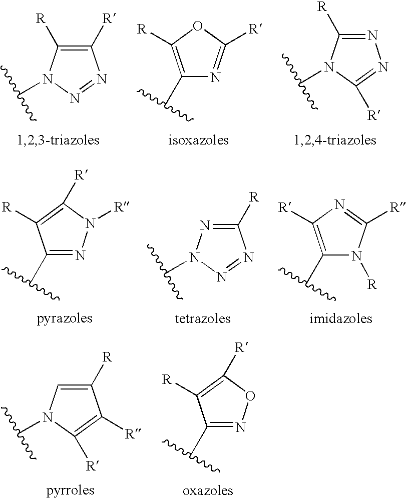

in which structures R, R′, and R″ independently represent H, an aromatic group ZZ, C₁-C₆ linear or branched alkyl or alkenyl group; the aromatic group ZZ being of the form:

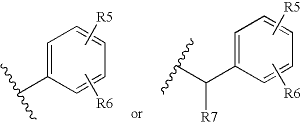

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxide group, sulfate, cyano, hydroxy or trifluoromethyl group

### IN:

R1=H, OR_{z}, NRₐR_{z}, CN, CHO, (C═O)OR_{z}, O(C═O)R_{b}, O(C═O)NHR_{f}, SR_{z}, ═O, ═S where R_{z}═H, C₁-C₆ linear or branched alkyl or alkenyl group, or an aromatic group ZZ, and Rₐ═H, C₁-C₆ linear or branched alkyl or alkenyl group, or an aromatic group ZZ, and R_{b}═H, C₁-C₂₂ linear or branched alkyl or alkenyl group, or an aromatic group ZZ, or R_{b} corresponds to the partial structure YX shown below, and R_{f}═H, C₁-C₆ linear or branched alkyl or alkenyl group, or an aromatic group ZZ or R_{f} corresponds to the partial structure YX shown below;
R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{f}, CH₂SR_{z}, CH═O, CH═S where R_{z}═H, C₁-C₆ linear or branched alkyl or alkenyl group, or an aromatic group ZZ, and Rₐ═H, C₁-C₆ linear or branched alkyl or alkenyl group, or an aromatic group ZZ, and R_{b}═C₁-C₂₂ linear or branched alkyl or alkenyl group, or an aromatic group ZZ, or R_{b} corresponds to the partial structure YX shown below, and R_{f}═H, C₁-C₆ linear or branched alkyl or alkenyl group, or an aromatic group ZZ or R_{f} corresponds to the partial structure YX shown below;

### R3=CH₂═C—CH₃ or CH₃—CH—CH₃;

### X₁₀═X₁₁═H;

X₁₂═X₁₃=“absent”;
a, b, c, and d independently represent a single or a double bond; and
e=“absent”; and said aromatic group ZZ is of the form:

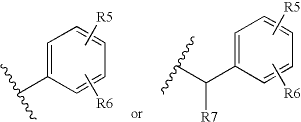

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl group; and
the partial structure R_{f} or R_{b} is of the form YX:

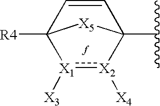

where R4=H or a C₁-C₂₀ linear or branched alkyl or alkenyl group, or an aromatic group ZZ;
X₅=“absent”, C, O, N, or S;
X₁—X₂ forms a cyclic partial structure of the form:
X₁—(X₃═X₆)—X₇—(X₄═X₈)—X₂ where

### X₁═X₂═C or N;

### X₃═X₄═C;

X₆═X₈═O, S or “absent”;
X₇═C, O, S, or N—X₉ where X₉═H, C₁-C₆ linear or branched alkyl or alkenyl group, or an aromatic group ZZ; and
f=a single or a double bond

### IO:

R1=H, OR_{z}, NRₐR_{z}, CN, CHO, (C═O)OR_{z}, O(C═O)R_{b}, O(C═O)NHR_{f}, SR_{z}, ═O, ═S where R_{z}═H, C₁-C₆ linear or branched alkyl or alkenyl group, or an aromatic group ZZ, and Rₐ═H, C₁-C₆ linear or branched alkyl or alkenyl group, or an aromatic group ZZ, and R_{b}═H, C₁-C₂₂ linear or branched alkyl or alkenyl group, or an aromatic group ZZ, or R_{b} corresponds to the partial structure YX shown below, and R_{f}═H, C₁-C₆ linear or branched alkyl or alkenyl group, or an aromatic group ZZ or R_{f} corresponds to the partial structure YX shown below;
R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{f}, CH₂SR_{z}, CH═O, CH═S where R_{z}═H, C₁-C₆ linear or branched alkyl or alkenyl group, or an aromatic group ZZ, and Rₐ═H, C₁-C₆ linear or branched alkyl or alkenyl group, or an aromatic group ZZ, and R_{b}═C₁-C₂₂ linear or branched alkyl or alkenyl group, or an aromatic group ZZ, or R_{b} corresponds to the partial structure YX shown below, and R_{f}H, C₁-C₆ linear or branched alkyl or alkenyl group, or an aromatic group ZZ or R_{f} corresponds to the partial structure YX shown below;

### R3=CH₂═C—CH₃ or CH₃—CH—CH₃;

### X₁₀═X₁₁═H;

X₁₂═X₁₃=“absent”;
a, b, c, and d independently represent a double or a single bond; and
e=“absent”; and said aromatic group ZZ is of the form:

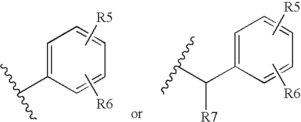

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl; and
the partial structure R_{f} or R_{b} is of the form YX:

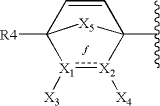

where R4=H or a C₁-C₂₀ linear or branched alkyl or alkenyl group, or an aromatic group ZZ;
X₅=“absent”, C, O, N, or S;

### X₁═X₂═C or N; and

X₃═X₄═R_{g}, (C═O)OR_{g} or (C═O)NHR_{g} where R_{g}═H, C₁-C₆ linear or branched alkyl or alkenyl group; and
f=a single or a double bond

### IP:

R1=H, OR, NRₐR_{z}, CN, CHO, (C═O)OR_{z}, O(C═O)R_{b}, O(C═O)NHR_{z}, SR_{z}, ═O, ═S where R_{z}═H, C₁-C₆ linear or branched alkyl or alkenyl group, or an aromatic group ZZ, and Rₐ═H, C₁-C₆ linear or branched alkyl or alkenyl group, or an aromatic group ZZ, and R_{b}═H, C₁-C₂₂ linear or branched alkyl or alkenyl group, or an aromatic group ZZ;
R2=CH₂OR_{b}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{z}, CH₂SR_{z}, CH═O, CH═S where R_{z}═H, C₁-C₆ linear or branched alkyl or alkenyl group, or an aromatic group ZZ, and Rₐ═H, C₁-C₆ linear or branched alkyl or alkenyl group, or an aromatic group ZZ, and R_{b}═C₁-C₂₂ linear or branched alkyl or alkenyl group, or an aromatic group ZZ;

### R3=CH₂═C—CH₃ or CH₃—CH—CH₃; and

group ZZ is of the form:

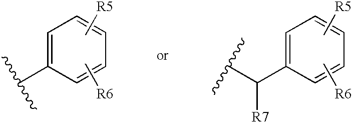

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl;
at X₁₀—X₁₁, a cyclic or heterocyclic partial structure having the form
X₁₀(X₁₂═X₁₄)—X₁₅—(X₁₃═X₁₆)—X₁₁ may be present where

### X₁₀═X₁₁═C or N;

### X₁₂═X₁₃═C;

X₁₄═X₁₆═O, S or “absent”;
X₁₅═C, O, S, or N—X₁₇ where X₁₇═H, a C₁-C₆ linear or branched alkyl or alkenyl group, or an aromatic group ZZ; and a, b, c, d and e independently represent double or single bonds

### IQ:

R1=H, OR_{z}, NRₐR_{z}, CN, CHO, (C═O)OR_{z}, O(C═O)R_{b}, O(C═O)NHR_{z}, SR_{z}, ═O, ═S where R_{z}═H, C₁-C₆ linear or branched alkyl or alkenyl group, or an aromatic group ZZ, and Rₐ═H, C₁-C₆ linear or branched alkyl or alkenyl group, or an aromatic group ZZ, and R_{b}═H, C₁-C₂₂ linear or branched alkyl or alkenyl group, or an aromatic group ZZ;
R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{z}, CH₂SR_{z}, CH═O, CH═S where R_{z}═H, C₁-C₆ linear or branched alkyl or alkenyl group, or an aromatic group ZZ, and Rₐ═H, C₁-C₆ linear or branched alkyl or alkenyl group, or an aromatic group ZZ, and R_{b}═H, C₁-C₂₂ linear or branched alkyl or alkenyl group, or an aromatic group ZZ;

### R3=CH₂═C—CH₃ or CH₃—CH—CH₃; and

said aromatic group ZZ is of the form:

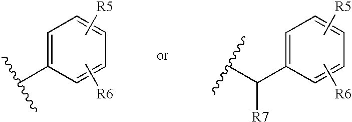

where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl; and
at X₁₀—X₁₁, a novel cyclic or heterocyclic partial structure may be present where X₁₀═X₁₁═C or N;
X₁₂═X₁₃═R, (C═O)OR or (C═O)NHR where R═H or a C₁-C₆ linear or branched alkyl or alkenyl group, or an aromatic group ZZ; and
a, b, c, d and e independently represent double or single bonds.

Preferable compounds derived from betulin for the inventive composition include compounds selected from the group consisting of betulin 3,28-C₁₋₈-dialkenylsuccinic acid diester, betulin 28-carvacrolacetic acid ester, betulin 3-acetate-28-mesylate, betulin 28-N-acetylanthralinic acid ester, betulin 3,28-dioxime, betulin 28-oxime, betulin 3-acetoxime-28-nitrile, betulin 28-acetic acid methylester, 20,29-dihydrobetulonic acid, betulonic acid, 28-aspartateamide dimethylester of betulonic acid, betulin 28-N-acetylanthranilic acid ester, Diels-Alder adduct of 3β-28-diacetoxylupa-12,18-diene and urazole, Diels-Alder adduct of 3β-28-diacetoxylupa-12,18-diene and 4-methylurazole, Diels-Alder adduct of 3β-28-diacetoxylupa-12,18-diene and p-fluoro-4-phenylurazole, Diels-Alder adduct of 3β-28-diacetoxylupa-12,18-diene and m-methoxy-4-phenylurazole, Diels-Alder adduct of 3β-28-diacetoxylupa-12,18-diene and 1-naphthylurazole, and Diels-Alder adduct of 3β-28-diacetoxylupa-12,18-diene and 1,3-dioxol-5-ylurazole.

Among the compounds derived from betulin, considerable antibacterial activity was found for betulonic acid and 28-N-acetylanthranilic acid ester of betulin already at a concentration of 1 μg/ml as shown by the examples below.

Novel betulin derivatives include amino acid, anthranilic acid, chrysanthemic acid, ornithine acid, cinnamic acid, retinolic acid, and trimethyl glycine, alpha-terpineol, verbenol, thymol, carvacrol, menthol, cinnamic alcohol, curcumin, eugenol, borneol, isoborneol, longifolol, isolongifolol, globulol, epiglobulol, sedrol, and episedrol derivatives of betulin, betulonic acid or betulinic acid.

Moreover, novel compounds of the invention include products and derivatives thereof obtained with subsequent reactions of betulin 29-olefins such as with an alkylation reaction or an ene reaction, such as betulin succinate, phenol, and polyphenol derivatives.

Here, useful compounds derived from betulin according to the invention also refer to salts, and particularly pharmaceutically acceptable salts thereof. Pharmaceutically acceptable salts are obtained from compounds by known methods using bases or acids.

In addition to the betulonic acid and the optional betulin derivative, the composition according to the invention comprises one or more constituent(s) or excipient(s) selected from the group of additives, fillers, carriers, vectors, surfactants, solvents, UV protectors, antioxidants, preserving agents, colouring agents, alcohols, waxes, oils, fats, perfumes, thickeners. The constituents and amounts thereof are selected according to the final product being prepared.

Further, the composition may comprise one or more pharmaceutically and/or cosmetically active agent(s) such as cortisone, cortisone derivative, vitamin, or a plant extract.

The formulation of the invention for topical use may be in liquid or semisolid form, or a foam, shampoo, spray, patch, stick, spreadable paste or sponge. Preferable formulations include liquid or semisolid formulations.

Various liquid formulations for topical use are preparations with varying viscosities to be applied on skin or nails for the provision of a local effect, or an effect after penetration of the skin. Said formulations are for instance solutions, emulsions, microemulsions, lotions, or suspensions that may contain one or more active agent(s) in a suitable vehicle. Said formulations may be in the form of aqueous, aqueous/alcoholic or oily solutions; in the form of dispersions of the lotion or serum type; in the form of oil-in-water emulsions obtained by dispersing a fatty phase in an aqueous phase, or vice versa, that is water-in-oil emulsions. Said formulations may also contain suitable microbicidal preserving agents, or antioxidants and other additives such as stabilizing and emulsifying agents, and thickeners.

Semisolid formulations for topical administration are used for the local delivery of the active agent or for the delivery thereof through the skin, or for emollifying or protecting purposes. The preparations consist of a simple or mixed base, and typically one or more active agent(s) dissolved or dispersed therein. According to the composition, the base may have an influence on the activity of the preparation. Said preparations may contain suitable additives such as antimicrobial preserving agents, antioxidants, stabilizing agents, emulsifying agents, thickeners and penetration promoters. Semisolid preparations for topical use may be of different types: cremes, gels, ointments, pastes and masks.

Lotions and cremes may be produced by conventional homogenizing methods known to those skilled in the art, but, however, also a microfluidization method is useful wherein aqueous and oil phases are mixed together in a high pressure homogenizer, thus considerably reducing the droplet size of the emulsion, to a value of about 1/400 of the droplet size in cremes and lotions prepared without high pressures. Using microfluidization, it is possible to prepare fine, stable cremes and lotions containing effective amounts of betulonic acid, or betulonic acid and other betulin derivatives, without using traditional emulsifying agents or surfactants.

Ointments consist of a base with a single phase containing solids or liquids dispersed therein. Typical bases to be used in formulations of hydrophilic ointments include hard, liquid and light liquid paraffins, vegetable oils, animal fats, synthetic glycerides, waxes and liquid polyalkyl siloxanes. Typical emulsifiers in ointments where water is emulsified include wool alcohols, sorbitan esters, monoglycerides and fatty alcohols, sulfate fatty alcohols, polysorbates, macrogel cetostearyl ether or fatty acid esters containing makrogols, whereas in hydrophilic ointments, mixtures of liquid and solid macrogols are used as emulsifiers.

The purpose of the carrier is to enhance the distribution of the composition when applied on the skin. Besides or instead of water, other useful carriers include liquid or solid emollients, solvents, emulsifiers, humectants, thickeners, powders, surface active agents, moisturizing agents, peeling agents, stabilizing agents, lubricants, chelating agents, agents enhancing penetration through the skin, fillers, perfumes and aromas, odour reducers, colouring agents and opacifying agents.

According to a preferable embodiment, said betulonic acid or betulin derivative is a powder used either as such, or as a dispersion or solution.

Suitable emollients include e.g. mineral oil, vaseline, paraffin, cerecine, ozocerite, microcrystalline wax, perhydrosqualene dimethylpolysiloxanes, methylphenyl-polysiloxanes, silicone-glycol-copolymers, triglyceride esters, acetylated monoglycerides, ethoxylated glycerides, alkylesters of fatty acids, fatty acids and alcohols, lanolin and lanolin derivatives, esters of polyhydric alcohols, sterols, derivatives of beeswax, polyhydric alcohols and polyethers, and fatty acid amides. Other suitable emollients are presented in Sgarin, Cosmetics, Science and Technology, 2. edition, vol. 1, pages 32-43 (1972).

Cationic, anionic, non-ionic, or amphotheric emulsifying agents or mixtures thereof may be used. Exemplary non-ionic emulsifying agents include commercially available sorbitans, alkoxylated fatty alcohols and alkylpolyglycosides. Anionic emulsifiers include soaps, alkyl sulfates, monoalkyl and dialkyl phosphates, alkyl-sulfonates and acyl isothionates. Other suitable emulsifiers are described in McCutcheon, Detergents and Emulsifiers, North American Edition, pages 317-324 (1986).

Preserving agents useful in the present formulations include alkanols, particularly ethanol and benzylic alcohol, parabens, sorbates, urea derivatives and isothiazolinones.

Suitable thickeners include starch derivatives, agar-agar, pectin, xantane gum, xanthane gum resistant to saline, cellulose derivatives such as hydroxypropyl cellulose and hydroxyethyl cellulose, carbopol and acacia gum, Sepigel 305 (available from Seppic Co., France), vec gum and magnesium aluminium silicate.

Urea, PCA, amino acids, some polyols, and other hygroscopic compounds may be mentioned as exemplary suitable humectants.

Preserving agents useful in the present formulations include alkanols, particularly ethanol and benzylic alcohol, parabens, sorbates, urea derivatives and isothiazolinones.

Suitable solvents include water and organic solvents, for instance alcohols selected from the group consisting of monoalcohols, glycols, diols and polyols. Suitable glycols to be used in the invention include glycerine, propylene glycol, butylene glycol, pentylene glycol (1,2-pentanal diol), neopentyl glycol (neopentane diol), caprylyl glycol (1,2-octane diol), ethoxy diglycol, butylene glycol monopropionate, diethylene glycol monobutylether, PEG-7 methylether, octacosanyl glycol, arachidyl glycol, benzyl glycol, cetyl glycol (1,2-hexane diol), C₁₄₋₁₈ glycol, C₁₅₋₁₈ glycol, lauryl glycol (1,2-dodecane diol), butoxy glycol, 1,10-decanediol, ethylhexanediol, or any mixtures thereof, without being limited to said compounds.

Suitable UV protectors include photoactive agents. A photoactive agent may be a UV filter, UV-A filter, UV-B filter, or a combination thereof. The UV filter is selected from the group consisting of p-aminobenzoic acid, salts and derivatives thereof such as ethyl, isobutyl, and glyceryl esters, and p-dimethylaminobenzoic acid; anthranilates (methyl, menthyl, phenyl, benzyl, phenylethyl, linalyl, terpinyl and cyclohexenyl esters of o-aminobenzoates); salicylates (octyl, amyl, phenyl, benzyl, menthyl (homosalate), glyceryl, and dipropylene glycol esters); derivatives of cinnamic acid (menthyl and benzyl esters, alpha-phenyl cirmamonitrile; butylcinnamoyl pyruvate); derivatives of dihydroxy cinnamic acid (umbellipherone, methyl umbellipherone, methyl acetoumbellipherone); camphoric derivatives (3-benzylidene, 4-methylbenzylidene, polyacrylamido methylbenzylidene, benzalconium methosulfate, benzylidene camphorsulfonic acid, and terephthalylidene dicamphorsulfonic acid); derivatives of trihydroxycinnamic acid (esculetin, methylesculetin, daphnetin, and esculin and daphnin glucosides); hydrocarbons (diphenylbutadien, stilben); dibenzal aceton and benzal acetophenon; naphtole sulfonates (sodium salts of 2-naphtole-3,6-disulfonic acid and 2-naphtole-6,8-disulphonic acid); dihydroxynaphtoic acid and salts thereof; o- and p-hydroxydiphenyl disulfonates; coumarin derivatives (7-hydroxy, 7-methoxy, 3-phenyl); diazoles (2-acetyl-3-bromo-indazole, phenylbenzoxazole, methylnaphtoxazole, various arylbenzothiazoles); chinine salts (bisulfate, sulfate, chloride, oleate, and tannate salts); chinoline derivatives (8-hydroxychinoline salts, 2-phenylchinoline); hydroxy or methoxysubstituted benzophenones; uric or vilouric acid; tannic acid and derivatives thereof; hydrochinone; benzophenones (oxybenzone, sulisobenzone, dioxybenzone, benzoresorcinole, 2,2′,4,4′-tetrahydroxy benzophenone, 2,2′-dihydroxy-4,4′-dimethoxybenzophenone, octabenzone); dibenzoylmethane derivatives, avobenzone, 4-isopropyldibenzoyl methane, butylmethoxy dibenzoylmethane, 4-isopropyl dibenzoylmethane, octocrylen, drometrizole trisoloxane, and metan oxides (titanium dioxide, zinc oxide, cerium dioxide).

Compositions of the invention may be prepared by mixing the constituents of the composition. The present betulin derived compounds may be emulsified, dissolved, or mixed in water, or in adjuvants and vehicles used in the art using known mixing and production processes and additives such as surfactants, emulsifiers, dispersants, and solvents, optionally while heating. Suitable vehicles include alcohols, polyols, and polyol esters, various gels and fats, vegetable oils and solid vehicles not hazardous to health such as starch, chitosan and cellulose and derivatives thereof, kaolin, talcum, and the like. Suitable vegetable oils include rapeseed, colza, tall, sunflower, palm, soybean, arachis, mandelic, poppy seed, corn, and olive oils.

Alternatively, a finely divided powder having a predetermined particle size distribution is produced from betulon and an optional betulin derivative by grinding as such or together with one or several of the above component(s), followed by the conversion of said powder into a solid powder, dispersion, emulsion, suspension, or a solution by means of a suitable solvent or vehicle to be selected among the above components and optionally with heating, to be mixed as desired with other components of the composition by methods and apparatuses known as such in the art.

Alternatively, birch bark or a fraction extracted from birch bark mainly containing betulin may be reactively ground under oxidizing conditions for instance by adding a low catalytic amount (0.2 to 2%) of hydrogen peroxide to the mixture, thus yielding betulonic acid and derivatives thereof in a powdery form that may be further used as presented above for the preparation of compositions.

It is also possible to prepare concentrates containing between 0.1 and 50% by weight of betulonic acid and optionally between 0.1 and 50% by weight of one or more betulon derivative(s) and at least one of the above components. Final products of the invention may be then prepared from the concentrate using known mixing methods.

Compositions of the invention are particularly suitable for use on the skin as a sun protection products since betulon and betulon derivatives act in the products as effective non-cytotoxic preserving agents, the performance of which may still be enhanced with glycols such as pentylene glycol and dioctyl glycol themselves microbicidal agents, and thus an activity with a wider spectrum may be provided even without preserving agents typically used in the art. Moreover, the compounds act as efficient UV-filters since part of the compounds may remain as a solid powder, thus providing slow dissolution, and slow effect of the compounds on the skin, and a coating made of particles of the active agent on the skin. The vehicle is selected to optimize the penetration of the active agent into the skin (may be evenly applicated, and moisturizing without a grassy feel).

Compositions according to the invention are also well suited for coloured cosmetic products, lipsticks, skin care products, creams, emulsions, sprays, hair care products, products for animals, such as sun protection products for bovine udder since penetration of betulonic acid into the skin may be prevented and thus no undesirable compounds may pass into milk.

Solubility/wettability of betulonic acid and betulin derivatives may be improved and penetration thereof into the skin may be controlled as desired by lactic acid and oligomers thereof.

In several betulin derivatives, substituents present are naturally occurring substances or known compounds with low toxicity, and thus said compounds are safe and environmentally acceptable. In addition, solubility and/or emulsifiability of many of these compounds in solvents and vehicles used in cosmetic and pharmaceutical industries is improved.

It was also surprisingly found that the active compound is released by some betulin derivatives in a controlled manner during a long period of time. This enables efficient desirable administration of the products of the invention.

It was surprisingly found that betulonic acid 2 may be used as an efficient bactericidal agent.

Substituents present in the novel betulin derivatives presented above are often derived from naturally occurring substances or known compounds with low toxicity, or both, or said substituents are typical heterocyclic moieties. Several of these compounds derived from betulin are environmentally acceptable compounds having only weak potential negative effects on the user and environment, said negative effects being also more predictable that those of synthetic compounds. Decomposition of compounds derived from betulin typically yields betulin or acid derivatives thereof, and further, constituents of substituents. Decomposition pathways of constituents, such as natural substances, present as structural moieties in the compounds and products thus generated are well known. Moreover, the toxicity of betulin derivatives is low as demonstrated by the cytotoxicity studies performed in the examples below.

Using compositions of the invention, it is possible to prevent potential microbial infections or contaminations, and simultaneously protect the skin against detrimental effects of UV light.

Betulonic acid and compounds derived from betulin are typically biodegradable, like betulin. Moreover, no bacteria with acquired resistance to betulin are known, and thus such acquired resistance to the present betulin derivatives or betulonic acid is not expected.

Particularly betulin derivatives of the invention having alkyl groups with long chains as substituents have a superior emulsifiability and/or solubility and/or miscibility in water or alcohols, polyols or polyol esters, various gels and fats, or vegetable oils or fatty acid derivatives thereof.

The solution according to the invention has several advantages. Being nontoxic, the betulin derivatives defined above and betulonic acid are very useful in pharmaceutical and cosmetic applications for humans and animals. The compounds are biodegradable leaving no detrimental decomposition residues in nature. In addition, only targeted organisms are very specifically affected by the compounds. According to the targeted application, the selectivity and decomposition rate of the agent may be controlled by substituents of betulin. If necessary, a compound decomposing more slowly, releasing the active component during decomposition, may be prepared, resulting in a uniform activity for a longer period or so-called “modified/controlled release” activity.

Betulin derivatives of the invention described above may be produced by methods I-XIV presented below.

### Method I

Betulin esters of the type IB or IFb described above may be produced by reacting 1 mol of betulin with 0.8-1.5 moles, preferably 1-1.2 moles of a C₄-C₂₂ alkyl or alkenyl derivative of maleic anhydride in the presence of imidazol (1-7 moles, preferably 3-5 moles), and a solvent at 0 to 100° C., preferably at 20 to 70° C., for 5 to 100 hours, preferably 10 to 50 h. C₁₋₈ alkenyl succinic anhydride (ASA) is preferably used. N-methyl-2-pyrrolidon (NMP), N,N-dimethylformamide (DMF), dimethylsulfoxide (DMSO), 1,4-dioxane, diethyl ether, tetrahydrofuran (THF), acetone, ethyl acetate, hydrocarbons and/or chlorinated hydrocarbons or mixtures thereof, preferably NMP, may serve as the solvent. After completion of the reaction, the reaction mixture is allowed to cool to room temperature, followed by separation of the product for instance by pouring the mixture into water, decanting, dissolving in a solvent, and then if necessary, washing the product with a diluted hydrochloric acid solution and water. The solvent is removed e.g. by evaporation to dryness, thus yielding desired betulin ester as the raw product that may be purified by crystallization, chromatography, or preferably by extraction using diethyl ether, tetrahydrofuran, 1,4-dioxane, 1,2-dimethoxy ethane, ethyl acetate, hydrocarbons and/or chlorinated hydrocarbons or mixtures thereof as the solvent. Esters corresponding to the structure IFb are obtained as the main product in case an excess of anhydride (1.6 to 5 moles, preferably 2 to 2.5 moles) is used, while the use of 1 to 1.2 moles of the anhydride yields esters corresponding to the structure IB.

### Method II

Betulin esters having structures of types IA, IC, ID, IE, IFa, IFd, and IFe described above may be produced from betulin (1 mol) and carboxylic acids (0.8 to 1.5 moles, preferably 1 to 1.2 moles) in the presence of N,N-dimethylamino pyridine (DMAP) (0.01 to 1 mol) and dicyclohexyl carbodiimide (DCC) (0.8 to 1.5 moles, preferably 1 to 1.2 moles), or N-(3-dimethylaminopropyl)-N′-ethylcarbodiimide hydrochloride (EDC) (0.8 to 1.5 moles, preferably 1 to 1.2 moles) and a solvent, by agitating at 0 to 60° C., preferably at 20 to 40° C. for 2 to 50 hours, preferably for 5 to 25 hours. The carboxylic acid is selected for different compound types as follows: IA: HO(C═O)Rᵢ where Rᵢ═C₁₁-C₂₂ linear or branched alkyl or alkenyl group; SIC: ornithine, nicotine, and N-acetylanthranilic acids or trimethyl glycine; ID: HO(C═O)CR_{X}(NHR_{Y}); R_{X}=alkyl, heteroalkyl, or arylalkyl group; R_{Y}═H or acyl group; and IE: a carboxymethoxy derivative of verbenol, terpineol, thymol, carvacrol, menthol, cinnamic alcohol, curcumin, eugenol, borneol, isoborneol, longifolol, isolongifolol, globulol, epiglobulol, sedrol, or episedrol; or chrysanthemic acid, cinnamic acid, or retinolic acid. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably dichloromethane, may serve as the solvent. After completion of the reaction, the reaction mixture is poured into water, organic layer is separated, followed by removing the solvent for instance by evaporation to dryness, thus yielding betulin ester as the raw product that may be purified if necessary by crystallization, chromatography, or extraction, preferably by extraction. Use of 0.8 to 1.5 moles of the carboxylic acid reagent results in compounds having the structures IA, IC, ID, IE or IFd while use of an excess of the carboxylic acid reagent (1.6 to 3 moles, preferably 2 to 2.5 moles) with dicyclohexyl carbodiimide (DCC) (1.6 to 3 moles, preferably 2 to 2.5 moles), or with N-(3-dimethylaminopropyl)-N′-ethylcarbodiimide hydrochloride (EDC) (1.6 to 3 moles, preferably 2 to 2.5 moles) yields compounds corresponding to structures IFa, IFc, IFd, or IFe. For the production of the compounds of the IE or IFe type, an acetic acid derivative of the alcohol used as starting material is first generated according to method V.

### Method III

Betulin esters having structures of types IA, IC, IE, IFa, IFc, and IFe described above may be produced from betulin (1 mol) with carboxylic acids (0.8 to 1.5 moles, preferably 1 to 1.2 moles) in the presence of a tetraisopropyl ortho titanate, tetrabutyl ortho titanate, p-toluenesulfonic acid monohydrate, or pyridine-p-toluenesulfonate catalyst (0.01 to 1 mol), or sulphuric acid or hydrochloric acid (1 to 6%, preferably 2 to 4%) and a solvent, by agitating at 80 to 160° C., preferably at 100 to 140° C. for 2 to 50 hours, preferably for 4 to 25 hours. The carboxylic acid is selected for different compound types as follows: IA: HO(C═O)Rᵢ where Rᵢ═C₁₁-C₂₂ linear or branched alkyl or alkenyl group; IC: ornithine, nicotine, and N-acetylanthranilic acids or trimethyl glycine; IE: a carboxymethoxy derivative of verbenol, terpineol, thymol, carvacrol, menthol, cinnamic alcohol, curcumin, eugenol, borneol, isoborneol, longifolol, isolongifolol, globulol, epiglobulol, sedrol, or episedrol; or chrysanthemic acid, cinnamic acid, or retinolic acid. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably toluene or xylene, may serve as the solvent. Water generated in the reaction is separated using a water separator tube, or vacuum. After completion of the reaction, the reaction mixture is poured into water, organic layer is separated, washed if necessary with a basic aqueous solution, preferably with an aqueous NaHCO₃ or Na₂CO₃ solution, followed by removing the solvent for instance by evaporation to dryness, thus yielding betulin ester as the raw product that may be purified if necessary by crystallization, chromatography, or extraction, preferably by extraction. Use of 0.8 to 1.5 moles of the carboxylic acid reagent results in compounds having the structures IA, IC, or IE while use of an excess of the carboxylic acid reagent (1.6 to 3 moles, preferably 2 to 2.5 moles) yields compounds corresponding to structures IFa, IFc, or IFe. For the production of the compounds of the IE or IFe type, an acetic acid derivative of the alcohol used as starting material is first generated according to method V.

### Method IV

Esters having structures of types IA, IC, ID, IE, IFa, Ifc, IFd, and IFe described above may be produced from betulin (1 mol) and carboxylic acids (0.8 to 1.5 moles, preferably 1 to 1.2 moles), first allowed to react with oxalyl chloride or thionyl chloride (1 to 10 moles, preferably 1 to 4 moles) without or in the presence of a solvent, by agitating at 0 to 80° C., preferably at 20 to 50° C. for 2 to 50 hours, preferably for 5 to 25 hours. The carboxylic acid is selected for different compound types as follows: IA: HO(C═O)Rᵢ where Rᵢ═C₁₁-C₂₂ linear or branched alkyl or alkenyl group; IC: ornithine, nicotine, and N-acetylanthranilic acids or trimethyl glycine; ID: HO(C═O)CR_{X}(NHR_{Y}); R_{X}=alkyl, heteroalkyl, or arylalkyl group; R_{Y}═H or acyl group; and IE: a carboxymethoxy derivatives of verbenol, terpineol, thymol, carvacrol, menthol, cinnamic alcohol, curcumin, eugenol, borneol, isoborneol, longifolol, isolongifolol, globulol, epiglobulol, sedrol, or episedrol; or chrysanthemic acid, cinnamic acid, or retinolic acid. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, the solvent is removed for instance by evaporation to dryness, if necessary, followed by purification of the desired acid chloride by crystallization, chromatography, or extraction, preferably by extraction. The acid chloride (0.8 to 1.5 moles, perferably 1 to 1.2 moles) thus obtained is reacted with betulin (1 mol), base (0.5 to 10 moles, preferably 1 to 5 moles) such as triethyl amine, tripropyl amine, diisopropylethyl amine, preferably triethyl amine in the presence of a solvent, or in the presence of the DMAP catalyst (0.01 to 1 mol), pyridine and solvent, or with a base (0.5 to 10 moles, preferably 1 to 5 moles) such as triethyl amine, tripropyl amine, diisopropylethyl amine, preferably triethyl amine, and pyridine by agitating at 0 to 80° C., preferably at 20 to 50° C. for 2 to 50 hours, preferably for 5 to 25 hours. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, betulin amide or betu-1M ester product is purified by crystallization, chromatography, or extraction, preferably by extraction, if necessary. Use of 0.8 to 1.5 moles of the acid chloride reagent results in compounds having the structures IA, IC, ID, or IE while use of an excess of the acid chloride reagent (1.5 to 3 moles, preferably 2 to 2.2 moles) yields compounds corresponding to structures IFa, We, IFd, or IFe. For the production of the compounds of the IE or IFe type, an acetic acid derivative of the alcohol used as starting material is first generated according to method V.

### Method V

For the production of betulin derivatives having structures of the IE and IFe type according to the methods II, III or IV, and betulin derivatives having structures of the IIa and IIb type according to the method VI, an acetic acid derivative of the alcohol is first generated as follows. Acetic acid derivative is produced by mixing an alcohol (1 mol) and chloroacetic acid (0.8 to 1.5 moles, preferably 1 to 1.2 moles) in water for 1 to 7 hours, preferably for 3 to 5 hours, at 100 to 150° C., preferably at 120-130° C., in the presence of lithium, potassium, sodium, or hydrides or hydroxides thereof (1.5 to 3 moles, preferably 1.8 to 2.2 moles), preferably sodium (Na), sodium hydride (NaH), or sodium hydroxide (NaOH). The alcohol is selected from the group consisting of verbenol, terpineol, thymol, carvacrol, menthol, cinnamic alcohol, curcumin, eugenol, borneol, isoborneol, longifolol, isolongifolol, globulol, epiglobulol, sedrol, and episedrol. The mixture is allowed to cool to room temperature, made acidic with concentrated hydrochloric acid, and extracter with a solvent. Hydrocarbons and/or chlorinated hydrocarbons, diethyl ether, tetrahydrofuran, 1,4-dioxane, 1,2-dimethoxy ethane, ethyl acetate, or mixtures thereof, preferably diethyl ether, may serve as the solvent. If necessary, the organic phase is washed with a basic aqueous solution, preferably with an aqueous NaHCO₃ or Na₂CO₃ solution. The solvent is removed for instance by evaporation to dryness, thus yielding a carboxymethoxy intermediate that may be purified if necessary by crystallization, chromatography, or extraction, preferably by extraction.

### Method VI

Derivatives of types IG, IH, II, and IJ described above may be produced from betulonic acid (1 mol) and natural alcohols (0.8 to 1.5 moles, preferably 1 to 1.2 moles), or amino acids (0.8 to 1.5 moles, preferably 1 to 1.2 moles), in the presence of a solvent and DMAP (0.01 to 1 moles) and DCC (0.8 to 1.5 moles, preferably 1 to 1.2 moles), or EDC (0.8 to 1.5 moles, preferably 1 to 1.2 moles), by agitating at 0 to 60° C., preferably at 20-50° C. for 2 to 50 hours, preferably for 5 to 25 hours. For the different compound types, the alcohol is selected as follows: IH: verbenol, terpineol, thymol, carvacrol, menthol, cinnamic alcohol, curcumin, eugenol, borneol, or isoborneol. For the different compound types, the amino acid is selected as follows: IG: HO(C═O)Rₜ where Rₜ═NHCHR_{X}COOY where Y═H, Na, K, Ca, Mg, C₁-C₄-alkyl group or NRₓ, where Rₓ═H, C₁-C₄-alkyl, benzyl, 4-hydroxybenzyl, —CH₂CH₂CH₂CH₂NH₂, 4-imidazolyl methyl, 3-indolyl methyl, or CH₃SCH₂ group; preferably dimethyl ester hydrochloride of aspartic acid, methyl ester hydrochloride of L-histidine, dimethyl ester hydrochloride of L-glutaminic acid, or methyl ester dihydrochloride of L-lysine. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, the desired betulonic acid amide or ester product (of the type IJa or IJb) may be purified by crystallization, chromatography, or extraction, preferably by extraction, if necessary. The betulonic acid amide or ester thus obtained may be reduced to the corresponding betulinic acid amide or ester product (of the type IG or 1H) if desired using sodium borohydride according to U.S. Pat. No. 6,280,778. After completion of the reaction, said betulinic acid amide or ester may be purified by crystallization, chromatography, or extraction, preferably by extraction, if necessary. Betulin derivatives of the IIa and IIb type are obtained by reacting the betulinic acid amide or ester thus obtained as described in the methods II, III or IV.

### Method VII

Compounds having structures of the types IG, IH, II, and IJ described above may be produced from betulonic acid (1 mol) by reacting with oxalyl chloride or thionyl chloride (1 to 10 moles, preferably 1 to 4 moles) without, or in the presence of a solvent by agitation at 0 to 80° C., preferably 20 to 50° C., for 2 to 50 hours, preferably for 5 to 25 hours. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, the desired acid chloride may be purified by crystallization, chromatography, or extraction, preferably by extraction, if necessary. Betulonic acid chloride thus obtained from the reaction (1 mol) is reacted with an amino acid (0.8 to 1.5 moles, preferably 1 to 1.2 moles), or an alcohol (0.8 to 1.5 moles, preferably 1 to 1.2 moles), with a base (0.5 to 10 moles, preferably 1 to 5 moles) such as triethyl amine, tripropyl amine, diisopropyl ethyl amine, pyridine, preferably triethyl amine in the presence of a solvent, or in the presence of the DMAP catalyst (0.01 to 1 mol), pyridine and solvent, or with a base (0.5 to 10 moles, preferably 1 to 5 moles) such as triethyl amine, tripropyl amine, diisopropylethyl amine, preferably triethyl amine, and pyridine by agitating at 0 to 80° C., preferably at 20 to 50° C. for 2 to 50 hours, preferably for 5 to 25 hours. For the different compound types, the amino acid is selected as follows: IG: HO(C═O)Rₜ where Rₜ═NHCHR_{X}COOY where Y═H, Na, K, Ca, Mg, C₁-C₄-alkyl group or NRₓ where Rₓ═H, C₁-C₄-alkyl, benzyl, 4-hydroxybenzyl, —CH₂CH₂CH₂CH₂NH₂, 4-imidazolyl methyl, 3-indolyl methyl, or CH₃SCH₂ group; preferably dimethyl ester hydrochloride of aspartic acid, methyl ester hydrochloride of L-histidine, dimethyl ester hydrochloride of L-glutaminic acid, and methyl ester dihydrochloride of L-lysine. For the different compound types, the alcohol is selected as follows: IH: verbenol, terpineol, thymol, carvacrol, menthol, cinnamic alcohol, curcumin, eugenol, borneol, isoborneol, longifolol, isolongifolol, globulol, epiglobulol, sedrol, episedrol, or eugenol. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, the reaction mixture is washed with dilute hydrochloric acid solution and water. The solvent is evaporated to dryness, and the reaction product (of the type IJa or IJb) is purified by crystallization, chromatography, or extraction, preferably by extraction, if necessary. The betulonic acid amide or ester product thus obtained may be reduced to the corresponding betulinic acid amide or ester product (of the type IG or III) using sodium borohydride according to U.S. Pat. No. 6,280,778. After completion of the reaction, the desired betulinic acid amide or ester is purifled by crystallization, chromatography, or extraction, preferably by extraction, if necessary. Betulin derivatives of the II type are obtained by reacting the betulinic acid amide or ester thus obtained as described in the methods II, III or IV.

### Method VIII

Compounds having structures of the type IK described above may be produced from betulin (1 mol) and aromatic compounds selected to have R_{z}═C₆H₅₋ₙ(OH)ₙ or C₆H₅₋ₙ₋ₘ(OH)ₙ(OCH₃)ₘ and n=0-5, m=0-5, n+m≦5 (4 to 20 moles) as the phenol residue in the IK group, in the presence of a polymeric acid catalyst, preferably a sulfonic acid derivative of polystyrene (0.1 to 1.5 g, preferably 0.5 to 1 g, 16 to 50 mesh) and a solvent. The reaction mixture is agitated in an inert atmosphere at 20 to 120° C., preferably at 75 to 110° C. for 1 to 5 hours, preferably for 2 to 4 hours. Water generated in the reaction is suitably separated using a water separating tube or vacuum. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably hydrocarbons and/or chlorinated hydrocarbons or an ether may serve as the solvent. After completion of the reaction, the mixture is allowed to cool to room temperature, filtered, the filtrate is washed with water, dried, and the solvent is separated. The betulin derivative thus obtained is purified by crystallization, chromatography, or extraction, preferably by extraction, if necessary.

### Method IX

Compounds having structures of the type IL described above may be produced from compounds having structures of the type IA or IFa prepared as described in the methods II, III, or IV, and maleic anhydride (0.8 to 10 moles, preferably 1 to 5 moles), in the presence of hydrochinone (0.05 to 0.5 moles, preferably 0.08 to 0.3 moles), and a solvent, or in a melt by heating the reaction mixture at 150 to 220° C., preferably at 160 to 180° C. for 1 to 5 hours, preferably for 2 to 4 hours. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof may serve as the solvent, preferably as a melt. After completion of the reaction, the desired product is purified by crystallization, chromatography, or extraction, preferably by extraction, if necessary. The maleic anhydride derivative of betulin thus obtained may be further converted into an imide or ester compound having the structure of the type IL using known methods.

### Method X

Betulin derivatives having structures of the types IM, IN, IO, IP and IQ described above may be produced by reacting betulin (1 mol) in the presence of triphenylphosphine (0.8 to 8 moles, preferably 2 to 5 moles), 3,3-dimethylglutaric imide (0.8 to 8 moles, preferably 2 to 5 moles), diethylazo dicarboxylate solution (0.8 to 8 moles, preferably 2 to 5 moles), and a solvent by agitating at 0 to 60° C., preferably at 20 to 40° C. for 2 to 5 hours, preferably for 5 to 25 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably tetrahydrofuran, may serve as the solvent. After completion of the reaction, the precipitate formed is filtered off. The solvent is removed for instance by evaporation to dryness, thus yielding 3-deoxy-2,3-dihydrobetulin as the raw product that may be purified by crystallization, chromatography, or extraction, preferably by extraction, if necessary.

### Method XI

Betulin derivatives having structures of the types IN and IO described above may be produced by reacting betulin (1 mol) with a Diels-Alder adduct (0.8 to 5 moles, preferably 1 to 2 moles), diphenylphosphoryl azide (DPPA) (0.8 to 5 moles, preferably 1 to 2 moles), and with a base, triethyl amine, tripropyl amine, diisopropylethyl amine, preferably triethyl amine (TEA) (0.8 to 5 moles, preferably 1 to 2 moles), in the presence of a solvent, by agitating at 0 to 150° C., preferably 60 to 120° C. for 1 to 48 hours, preferably for 2 to 24 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons or mixtures thereof, preferably toluene, may serve as the solvent. After completion of the reaction, the reaction mixture is washed with diluted aqueous basic solution, diluted acidic solution, water, if necessary, followed by removal of the solvent for instance by evaporating to dryness. 28-O-Diels-Alder adduct of betulin is obtained as the raw product that may be purified by crystallization, chromatography, or extraction, preferably by crystallization, if necessary. Use of an excess of the Diels-Alder adduct, diphenylphosphoryl azide (DPPA) and triethyl amine (1.5 to 3 moles, preferably 2 to 2.2 moles) results in 3,28-O-Diels-Alder diadduct of betulin.

Diels-Alder adducts may be produced from a C₅-C₂₂ diene acid (1 mol) that may be linear, branched, cyclic or heterocyclic comprising O, N or S as a hetero atom, preferably by reacting 2,4-pentadiene acid, sorbic acid, 2-furanoic acid or anthracene-9-carboxylic acid with a dienophile, preferably with 4-substituted triazolinedion, maleic anhydride, N-substituted maleimide, diethylazodicarboxylate, or dimethylacetylene dicarboxylate (0.5 to 5 moles, preferably 0.8 to 2 moles) in the presence of a solvent while agitating at 0 to 150° C., preferably at 20 to 120° C. for 1 to 48 hours, preferably for 2 to 24 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons or mixtures thereof, preferably toluene, may serve as the solvent. After completion of the reaction, the reaction mixture is washed with water, if necessary, followed by removal of the solvent by e.g. evaporation to dryness. A Diels-Alder adduct is obtained as the raw product that may be purified by crystallization, chromatography, or extraction, preferably by crystallization, if necessary.

### Method XII

Betulin derivatives having structures of the types IN and 10 described above may be produced by protecting the C28 hydroxyl group of betulin (1 mol) with a substituted methyl ether, substituted ethyl ether, substituted phenyl ether, silyl ether, ester, carbonate, or sulfonate using known methods, preferably with dihydropyran (DHP) (0.8 to 8 moles, preferably 1 to 2 moles), in the presence of pyridinium-p-toluene sulfonate (PPTS) (0.01 to 2 moles, preferably 0.05 to 0.5 moles) and a solvent while mixing at 0 to 60° C., preferably at 20 to 40° C. for 5 to 100 hours, preferably for 12 to 48 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably dichloromethane, may serve as the solvent. After completion of the reaction, the organic phase is washed with saturated aqueous solution of a base, and with water. The solvent is e.g. removed by evaporation to dryness yielding a betulin derivative as raw product having the C28 hydroxyl group protected with substituted methyl ether, substituted ethyl ether, substituted phenyl ether, silyl ether, ester, carbonate, or sulfonate, preferably with dihydropyran. The raw product, preferably betulin 28-tetrahydropyran ether may be purified by crystallization, chromatography, or extraction, preferably by extraction, if necessary.

Betulin derivative having the C28 hydroxyl group protected with substituted methyl ether, substituted ethyl ether, substituted phenyl ether, silyl ether, ester, carbonate, or sulfonate, preferably with dihydropyran (betulin 28-tetrahydropyran ether) (1 mol) and a Diels-Alder adduct (0.8 to 5 moles, preferably 1 to 2 moles) produced according to the method XI, diphenylphosphoryl azide (DPPA) (0.8 to 5 moles, preferably 1 to 2 moles), and a base, triethyl amine, tripropyl amine, diisopropyl ethyl amine, preferably triethyl amine (TEA) (0.8 to 5 moles, preferably 1 to 2 moles) are reacted in the presence of a solvent while mixing at 0 to 150° C., preferably at 60 to 120° C. for 1 to 48 hours, preferably 2 to 24 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably toluene, may serve as the solvent. After completion of the reaction, the reaction mixture is washed with a dilute basic solution, dilute acid solution, water, if necessary, followed by removal of the solvent e.g. by evaporation to dryness. As raw product, betulin derivative having the C28 hydroxyl group protected with substituted methyl ether, substituted ethyl ether, substituted phenyl ether, silyl ether, ester, carbonate, or sulfonate, preferably with dihydropyran, and having at C3 hydroxyl group a Diels-Alder adduct, preferably a Diels-Alder adduct of 2,4-pentadiene acid with 4-phenyl-1,2,4-triazolin-3,5-dion, is obtained. The raw product, preferably 3-O-Diels-Alder adduct of betulin 28-tetrahydropyran ether may be purified by crystallization, chromatography, or extraction, preferably by crystallization, if necessary.

C28 hydroxyl group of the betulin derivative having the C28 hydroxyl group protected with substituted methyl ether, substituted ethyl ether, substituted phenyl ether, silyl ether, ester, carbonate, or sulfonate, is deprotected using known methods, preferably the protecting group, tetrahydropyran, of the C28 hydroxyl of the 3-O-Diels-Alder adduct of 28-tetrahydropyran ether (1 mol) is cleaved using pyridinium-p-toluene sulfonate (PPTS) (0.02 to 1 mol, preferably 0.05 to 0.5 mol) by allowing said PPTS to react while agitating at 0 to 80° C., preferably at 20 to 40° C. for 24 to 240 hours, preferably 48 to 120 hours. NMP, DMF, DMSO, 1,4-dioxane, methanol, ethanol, 1-propanol, 2-propanol, diethyl ether, tetrahydrofuran, 1,2-dimethoxy ethane, acetone, ethyl acetate, hydrocarbons and/or chlorinated hydrocarbons, or mixtures thereof, preferably methanol or ethanol, may serve as the solvent. After completion of the reaction, the reaction mixture is diluted with an organic solvent, washed with a dilute aqueous solution of a base, dilute acidic solution, water, if necessary, followed by removal of the solvent for instance by evaporation to dryness. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably ethyl acetate, may serve as the solvent. Betulin 3-O-Diels-Alder adduct is obtained as raw product that may be purified by crystallization, chromatography, or extraction, preferably by crystallization.

### Method XIII

Heterocyclic betulin derivatives of the types IP and IQ described above may be produced by reacting betulin (1 mol) in the presence of an anhydride (1.6 to 5 moles, preferably 2 to 2.5 moles), N,N-dimethylamino pyridine (DMAP) (0.01 to 1 mol), a base, pyridine, triethyl amine, tripropyl amide, diisopropylethyl amine, preferably pyridine (1 to 100 moles, preferably 20 to 50 moles), and a solvent at 0 to 100° C., preferably at 20 to 50° C. for 5 to 100 hours, preferably 10 to 50 hours. The anhydride is preferably acetic anhydride, however, also other carboxylic anhydrides such as propionic anhydride, phthalic anhydride, or benzoic anhydride may be used. N-methyl-2-pyrrolidon (NMP), N,N-dimethylformamide (DMF), dimethylsulfoxide (DMSO), 1,4-dioxane, diethyl ether, tetrahydrofuran (THF), acetone, ethyl acetate, hydrocarbons and/or chlorinated hydrocarbons or mixtures thereof, preferably dichloromethane, may serve as the solvent. After completion of the reaction, the reaction mixture is washed, if necessary, with dilute hydrochloric acid solution, aqueous basic solution, and with water. Solvent is for instance removed by evaporation to dryness, giving 3,28-diester of betulin, preferably 3,28-diacetate of betulin as the raw product that may be purified by crystallization, chromatography, or extraction, preferably by extraction, if necessary.

The 3,28-diester of betulin (1 mol), preferably the 3,28-diacetate of betulin, may be isomerized to give 3β,28-diacetoxylup-18-enen in the presence of hydrochloric or hydrobromic acid, preferably hydrobromic acid (5 to 25%, preferably 10 to 15%), acetic acid (25 to 60%, preferably 35 to 50%), acetic anhydride (5 to 30%, preferably 10 to 20%), and a solvent at 0 to 60° C., preferably at 20 to 40° C. for 4 to 1200 hours, preferably for 10 to 24 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably toluene, may serve as the solvent. After completion of the reaction, the reaction mixture is washed, if necessary, with a basic aqueous solution and water, followed by removal of the solvent for instance by evaporation to dryness. 3,8,28-diacetoxylup-18-enen is obtained as raw product that may be purified by crystallization, chromatography, or extraction, preferably by crystallization, if necessary.

3β,28-diacetoxylup-18-enen (1 mol) may be epoxylated using hydrogen peroxide or a peracid, preferably ni-chloroperbenzoic acid (mCPBA) (0.8 to 3 moles, preferably 1 to 1.5 moles) in the presence of sodium carbonate, sodium hydrogen carbonate, sodium hydrogen phosphate, potassium carbonate, potassium hydrogen carbonate, potassium hydrogen phosphate, preferably sodium carbonate (1 to 15 moles, preferably 3 to 8 moles) and a solvent while agitating at 0 to 60° C., preferably at 20 to 40° C. for 0.5 to 10 hours, preferably 1 to 4 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably chloroform, may serve as the solvent. After completion of the reaction, the reaction mixture is washed, if necessary, with a basic aqueous solution and water, followed by removal of the solvent for instance by evaporation to dryness. 3β,28-diacetoxylup-18ε,19ε-epoxylupane is obtained as raw product that may be purified by crystallization, chromatography, or extraction, preferably by crystallization, if necessary.

3β,28-diacetoxylup-18ε,19ε-epoxylupane (1 mol) reacts to give 3β,28-diacetoxylupa-12,18-diene and 3β,28-diacetoxylupa-18,21-diene in the presence of p-toluenesulfonic acid (0.1 to 3 moles, preferably 0.3 to 1 moles) and acetic anhydride (0.5 to 5 moles, preferably 1 to 3 moles) and a solvent while agitating at 50 to 150° C., preferably at 90 to 130° C., for 0.5 to 12 hours, preferably for 2 to 5 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably toluene, may serve as the solvent. After completion of the reaction, the reaction mixture is washed, if necessary, with a basic aqueous solution and water, followed by removal of the solvent for instance by evaporation to dryness. 3β,28-diacetoxylupa-12,18-diene and 3β,28-diacetoxylupa-18,21-diene are obtained as raw products that may be purified by crystallization, chromatography, or extraction, preferably by crystallization, if necessary.

A heterocyclic Diels-Alder adduct may be produced from a mixture (1 mol) of 3β,28-diacetoxylupa-12,18-diene and 3β,28-diacetoxylupa-18,21-diene by reacting said mixture with a dienophile, preferably with 4-substituted triazolindion, maleic anhydride, N-substituted maleimide, diethylazodicarboxylate, or dimethylacetylene dicarboxylate (0.5 to 5 moles, preferably 0.8 to 2 moles) in the presence of a solvent while agitating at 0 to 150° C., preferably at 20 to 120° C., for 1 to 48 hours, preferably for 2 to 24 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably toluene, may serve as the solvent. After completion of the reaction, the reaction mixture is washed, if necessary, with water, followed by removal of the solvent for instance by evaporation to dryness. Heterocyclic Diels-Alder adduct of betulin is obtained as raw product that may be purified by crystallization, chromatography, or extraction, preferably by crystallization, if necessary.

### Method XIV

Substances having structures of the types IP described above may be produced by adding isocyanate (0.5 to 5 moles, preferably 0.8 to 1.5 moles) to ethylhydrazine (1 mol) in the presence of a solvent. The isocyanate R—N═C═O is selected from the group where R═H, C₁-C₆ linear or branched alkyl or alkenyl group or aromatic group ZZ of the formula

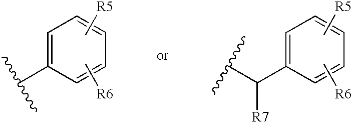

where R5, R6 and/or R7 may represent H, C₁-C₆ linear or branched alkyl or alkenyl group or C₁-C₆ linear or branched alkyl or alkenyl ether, R5-R6 forms a cyclic C₂-C₆-alkyl or alkenyl group, halogen (fluoro, chloro, bromo, iodo), nitro, carboxy, carboxyl, acetyl, R5-R6 forms a cyclic methylene dioxide group, sulfate, cyano, hydroxy, or trifluoromethyl. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably toluene, may serve as the solvent. The reaction mixture is agitated at 0 to 60° C., preferably at 0 to 40° C., for 0.5 to 12 hours, preferably for 1 to 5 hours, and 40 to 120° C., preferably at 60 to 100° C., for 0.5 to 12 hours, preferably for 1 to 5 hours. After completion of the reaction, the raw product formed is filtered and dried. The raw product, 4-substituted 1-carbethoxy semicarbazide may be purified by crystallization, chromatography, or extraction, preferably by extraction, if necessary.

Said 4-substituted 1-carbethoxy semicarbazide (1 mol) may be cyclized to give 4-substituted urazole by heating in an aqueous NaOH or KOH solution, preferably in aqueous KOH solution (1 to 10 M, preferably 2 to 6 M) at 40 to 100° C., preferably 50 to 80° C., for 0.5 to 6 hours, preferably 1 to 3 hours. The reaction mixture is filtered, followed by precipitation of the raw product with concentrated HCl solution, filtered and dried for instance in an oven or desiccator. The raw material, 4-substituted urazole, may be purified by crystallization, chromatography, or extraction, preferably by crystallization, if necessary.

Said 4-substituted urazole (1 mol) is oxidized using iodobenzene diacetate (0.5 to 6 moles, preferably 0.8 to 1.5 moles) in the presence of a solvent while agitating at 0 to 80° C., preferably at 20 to 40° C. for 0.1 to 4 hours, preferably 0.2 to 1 hours. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof, preferably tetrahydrofuran or dichloromethane, may serve as the solvent. A mixture of 3β,28-diacetoxylupa-12,18-diene and 3β,28-diacetoxylupa-18,21-diene produced according to the method XIII (0.2 to 2 moles, preferably 0.8 to 1.2 moles) is added to the reaction mixture, followed by agitating said reaction mixture at 0 to 60° C., preferably at 0 to 40° C., for 1 to 48 hours, preferably for 2 to 24 hours, and then, the solvent is removed e.g. by evaporation to dryness. The raw product, a Diels-Alder adduct of the 4-substituted urazole, may be purified by crystallization, chromatography, or extraction, preferably by crystallization.

The invention is now illustrated by the following examples without wishing to limit the scope thereof.

### EXAMPLES

### Example 1

### Preparation of the 28-C₁₋₈ alkylene succinic ester of betulin

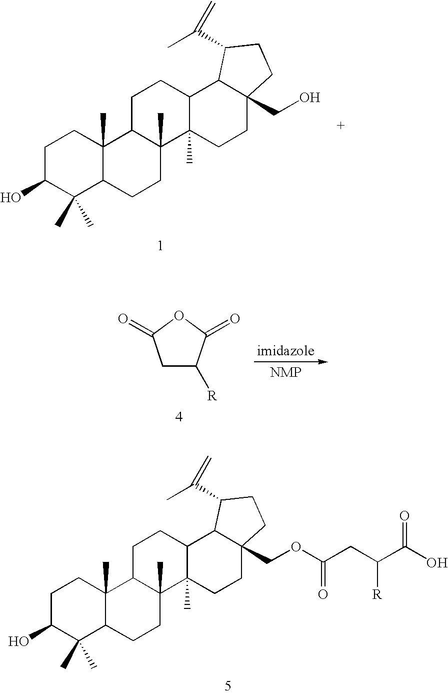

Imidazole (38.8 mmol) and C₁₋₈ alkylene succinic anhydride (ASA) 4 (11.6 mmol) were agitated in NMP (25 ml). Betulin 1 (9.7 mmol) was added, followed by further agitation at room temperature for 3 days. The organic phase was poured into water, decanted, dissolved in dichloromethane, and washed. The solvent was evaporated, thus yielding 28-C₁₋₈ alkylene succinic ester of betulin 5 with a yield of 73%.

### Example 2

### Preparation of the 3,28-C₁₋₈ alkylene succinic diester of betulin

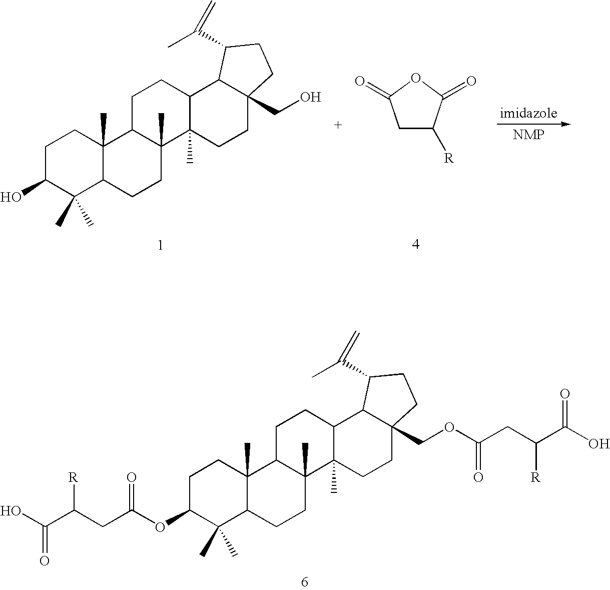

Imidazole (54.2 mmol) and C₁₋₈ alkylene succinic anhydride (ASA) 4 (32.5 mmol) were agitated in NMP (30 ml). Betulin 1 (13.5 mmol) was added, followed by further agitation at room temperature for 3 days. The organic phase was poured into water, decanted, dissolved in dichloromethane, and washed. The solvent was evaporated, thus yielding 3,28-C₁₋₈ alkylene succinic diester of betulin 6 (yield: 40%).

### Example 3

### Preparation of the 28-Carboxymethoxy Mentholester of Betulin

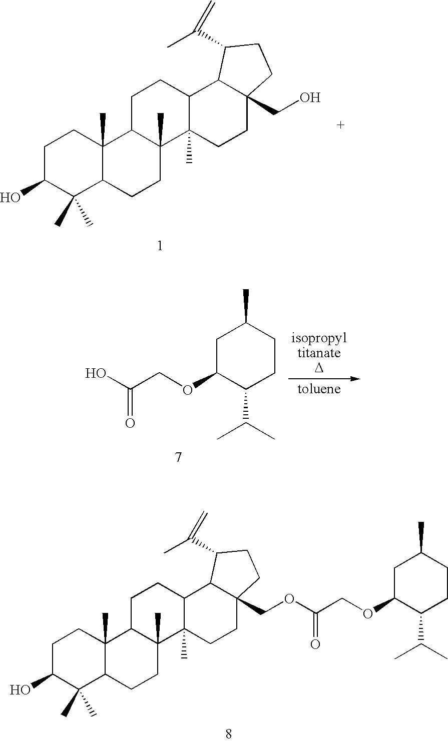

Betulin 1 (11.7 mmol) and menthoxyacetic acid 7 (11.7 mmol) were weighed in a flask, followed by the addition of toluene (120 ml) as the solvent. The mixture was heated to 120° C., and added with isopropyl titanate (1.4 mmol). The reaction mixture was refluxed for 3 h until water was separated to the water separation tube. The mixture was cooled to room temperature and the precipitate formed was filtered. The organic phase was washed and the solvent was evaporated, yielding 28-carboxymethoxy mentholester of betulin 8 (yield: 60%).

### Example 4

### Preparation of the 28-Carboxymethoxy Carvacrolester of Betulin

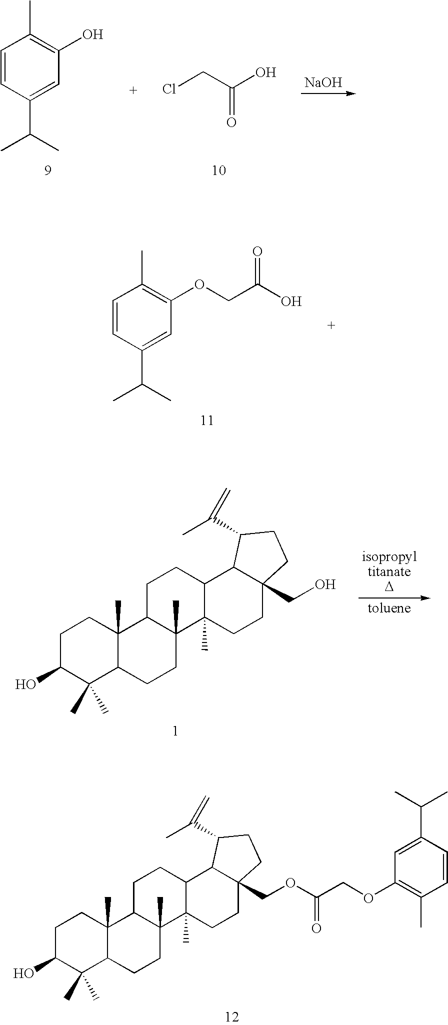

NaOH pellets dissolved in water (66.6 mmol) were added to a mixture of carvacrol 9 (33.3 mmol), chloroacetic acid 10 (33.3 mmol) and water (50 ml). The mixture was refluxed at 120° C. for 3 h. The mixture was cooled to room temperature and acidified with hydrochloric acid. The raw product was extracted with diethyl ether and washed with water. The solvent was evaporated, thus giving carvacrol oxyacetic acid 11 with a yield of 83%. The raw product was purified by dissolving in diethyl ether, followed by extraction with water and NaHCO₃ solution, which were pooled, acidified with hydrochloric acid and extracted with diethyl ether. The ether phase was dried, followed by evaporation of the solvent to dryness, thus giving carvacrol acetic acid 11 (yield: 45%). Betulin 1 (7.2 mmol) and carvacrol oxyacetic acid 11 (7.2 mmol) wer weighed into a flask, and toluene (80 ml) was added. The bath was heated to 160° C., and then isopropyl titanate (1.4 mmol) was added. The reaction mixture was refluxed for 6 h until all water was separated to the water separation tube. The mixture was cooled to room temperature and the precipitate formed was filtered. The organic phase was washed with NaHCO₃ solution and the solvent was evaporated. The raw product was recrystallized from boiling solution of cyclohexane and toluene. The solvent was evaporated to dryness, thus yielding 28-carboxymethoxy carvacrolester of betulin 12 (yield: 55%).

### Example 5

### Preparation of the 28-Cinnamon Alcohol Acetic Acid Ester of Betulin

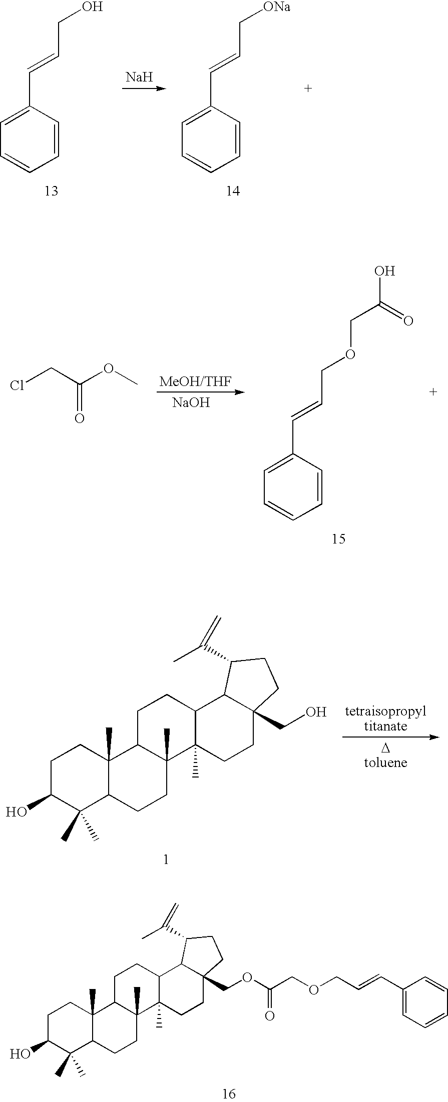

A mixture of sodium hydride (8.2 mmol) and tetrahydrofuran was added with cinnamon alcohon 13 (7.5 mmol), and agitation was continued for 1 h at room temperature. Methylchloroacetate (7.5 mmol) was added to the reaction flask, and agitation was continued for 24 hours. After completion of the reaction, the reaction mixture was diluted with diethyl ether, and then the organic phase was washed with water and dried. The solvent was evaporated to dryness, and the precipitate was dissolved in a solution of methanol and tetrahydrofuran. Sodium hydroxide solution (10.9 mmol) was added, and the reaction mixture was refluxed for 4 hours. The solvent was evaporated. Water was added to the flask, acidified with hydrochloric acid, and extracted with diethyl ether. The organic phase was washed with water, and the solvent was evaporated, thus giving cinnamic acid 15 with a yield of 23%. Betulin 1 (0.9 mmol) and cinnamic acid 15 (0.9 mmol) were weighed into a flask, and toluene (40 ml) was added. The bath was heated to 160° C., and then isopropyl titanate (0.2 mmol) was added. The reaction mixture was refluxed for 4.5 h until all water was separated to the water separation tube. The mixture was cooled to room temperature and the precipitate formed was filtered. The organic phase was washed with NaHCO₃ solution and the solvent was evaporated. The raw product was recrystallized from boiling solution of cyclohexane and toluene. After the mixture was cooled, the crystallized precipitate was filtered. The solvent was evaporated to dryness, thus giving 28-cinnamon alcohol acetic acid ester of betulin 16 (yield: 14%).

### Example 6

### Preparation of 28-Eugenolester of Betulonic Acid

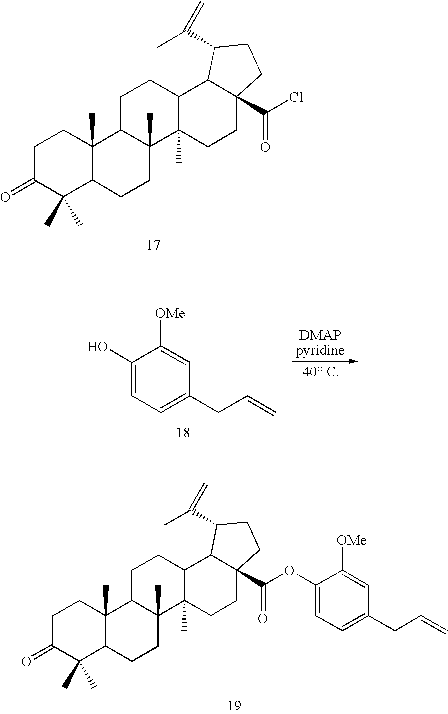

A mixture of betulonic acid chloride 17 (1.4 mmol) (prepared as described in example 12), eugenol 18 (1.1 mmol), DMAP (1.1 mmol), and pyridine was heated for 48 hours at 40° C. The reaction mixture was diluted with toluene, washed with dilute hydrochloric acid solution, and water and then dried over sodium sulfate. The solvent was evaporated, thus giving 28-eugenol ester of betulonic acid 19 (yield: 81%).

### Example 7

### Preparation of 28-Carboxymethoxythymol Ester of Betulin

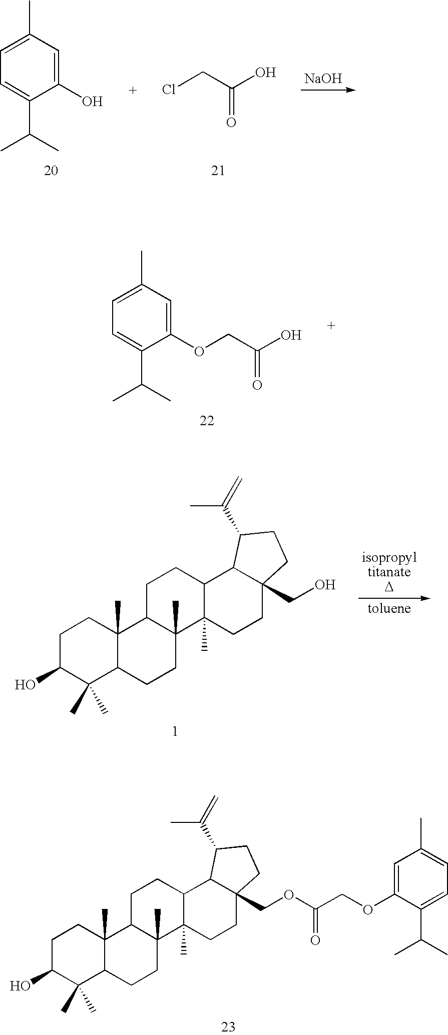

NaOH pellets dissolved in water (66.6 mmol) were added to a mixture of thymol 20 (33.3 mmol), chloroacetic acid 21 (33.3 mmol) and water. The mixture was refluxed at 120° C. for 3 h. The mixture was cooled to room temperature, acidified, extracted with diethyl ether and washed. The solvent was evaporated thus giving precipitated thymolacetic acid 22 with a yield of 29%. Betulin 1 (7.2 mmol), thymolacetic acid 22 (7.2 mmol), and toluene (80 ml) were heated to 160° C., followed by the addition of isopropyl titanate (1.4 mmol). The reaction mixture was refluxed for 4.5 h until all water was separated to the water separation tube. The mixture was cooled to room temperature and the precipitate formed was filtered. The organic phase was washed and the solvent was evaporated. The raw product was recrystallized from solution of cyclohexane and toluene (3.5:1), thus giving 28-carboxymethoxythymol ester of betulin 23 (yield: 61%).

### Example 8

### Preparation of 28-Chrysanthemate of Betulin

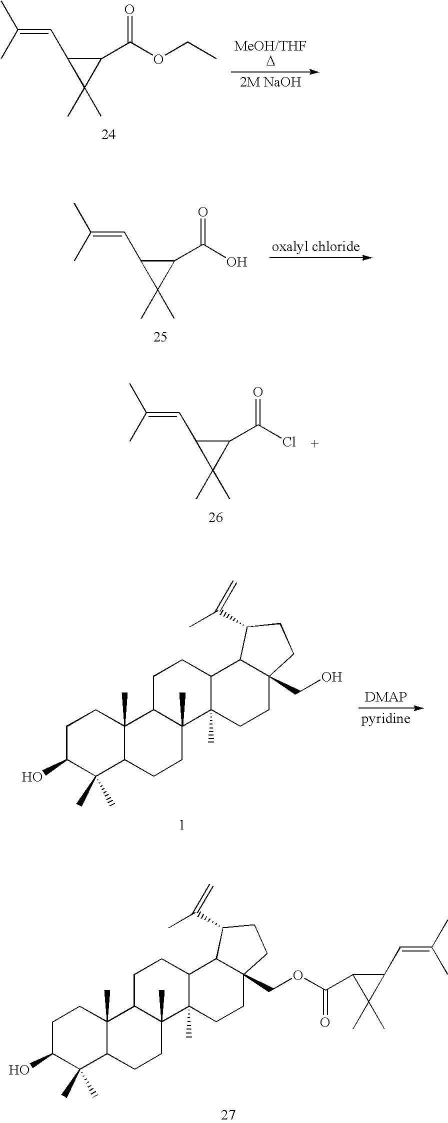

Ethyl chrysanthemate 24 (23.3 mmol) was mixed to a THF/MeOH solution (1:2) under an inert atmosphere. 2 M NaOH solution (93 ml) was slowly added to the mixture, and then, the reaction mixture was heated in a bath at 80° C. for 4 hours until no starting material was present as determined by TLC (hexane:ethyl acetate 6:1, 5% by volume of acetic acid). The solvent was evaporated, the raw product obtained was dissolved in water (400 ml) and extracted with diethyl ether. The aqueous phase was acidified with hydrochloric acid, and diluted with diethyl ether. The ether phase was washed and the solvent was evaporated in vacuum, thus giving chrysanthemic acid 25 (yield: 90%).

Chrysanthemic acid 25 (5.9 mmol) in anhydrous dichloromethane (30 ml) was added with oxalyl chloride (11.8 mmol) at room temperature under inert atmosphere. After six hours, the solvent was evaporated, and then the evaporation residue was taken up in dry dichloromethane, which was again evaporated. The procedure was repeated three times, thus giving chrysanthemic acid chloride 26 (yield: 81%).

Betulin 1 (0.9 mmol), chrysanthemic acid chloride 26 (1.1 mmol) and DMAP (0.9 mmol) were agitated in pyridine at 40° C. under inert atmosphere for 48 hours. EtOAc (100 ml) was added, organic phase was washed with water, the solvent was evaporated, and the residue was recrystallized in cyclohexane. 28-chrysanthemate of betulin 27 was obtained with a yield of 63%.

### Example 9

### Preparation of 28-Cinnamic Acid Ester of Betulin

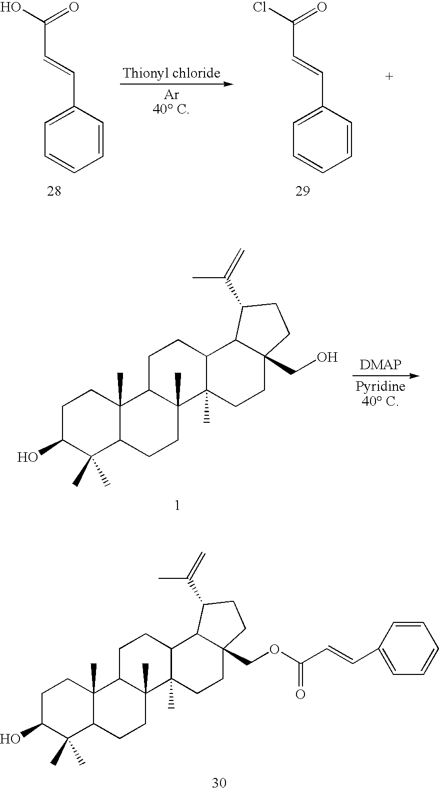

Cinnamic acid 28 (18.06 mmol) and thionyl chloride (180.6 mmol) were mixed under inert argon atmosphere at 40° C. for 24 hours. Solvent was evaporated under vacuum, followed by dissolving the evaporation residue twice in dichloromethane and evaporation, thus giving cinnamic acid chloride 29 (yield: 99%).

Betulin 1 (5.4 mmol) and cinnamic acid chloride 29 (5.6 mmol) were agitated in dry pyridine (80 ml) in the presence of DMAP (5.6 mmol) under inert argon atmosphere at 40° C. for 24 hours. Toluene (100 ml) was added, and the organic phase was washed. Solvent was evaporated, followed by purification of the raw product by recrystallization in a cyclohexane/toluene solution. 28-cinnamic acid ester of betulin 30 was obtained with a yield of 67%.

### Example 10

### Preparation of Fatty Acid Esters of Betulin

Betulin 1 (5 mmol) and a fatty acid (5 mmol) were weighed in a flask equipped with a water separation tube. Toluene and a catalytic amount of isopropyl titanate were added, followed by refluxing the reaction mixture in an oil bath for about 5 hours. The reaction mixture was allowed to cool to room temperature, the organic layer was washed with sodium hydrogen carbonate solution, separated, dried over sodium sulfate, and then the solvent was evaporated to dryness. The raw product obtained, betulin monoester, was purified by chromatography, if necessary. In case>2 equivalents of the fatty acids and 1 equivalent of betulin were used, also betulin diesters were obtained as the product as shown in table 1. Table 1 shows yields of the esterification reactions of betulin with fatty acids, and degrees of esterification.

TABLE 1

| | | Reflux | Total | C₃ degree of | C₂₈ degree of |
|---|---|---|---|---|---|
| | | time | yield | esterification | esterification |
| Fatty acid | Catalyst | (h) | (%) | (%) | (%) |
| Isostearic | Isopropyl | 3 | 81 | 0 | 40 |
| acid | titanate | | | | |
| Isostearic | p-toluene- | 4.5 | 99 | 10 | 95 |
| acid | sulfonic acid | | | | |
| Oleic acid | p-toluene- | 18.5 | 93 | 40 | 100 |
| | sulfonic acid | | | | |

### Example 11

### Preparation of 28-Amide Derivatives of Betulin

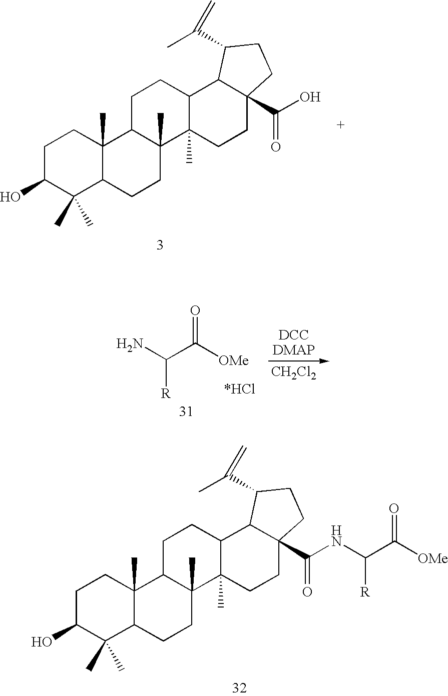

Betulinic acid 3 was prepared by oxidizing betulin 1 according to the document U.S. Pat. No. 6,280,778. Betulinic acid 3 (5 mmol) and aminoacid methyl ester hydrochloride 31 (5 mmol) were weighed in a flask and dissolved in dichoromethane. The flask was purged with argon, dichloromethane (5 mmol) and DMAP (2.5 mmol) were added and mixing was continued for 20 hours. The reaction mixture was diluted with ethyl acetate, washed with water, dried over sodium sulfate, and the solvent was evaporated to dryness. The betulinic acid amide 32 raw product may be purified by chromatography, if necessary. Reaction conditions and raw yields of the products are shown in Table 2.

TABLE 2

| Amino acid | Reaction time (h) | Total yield (%) |
|---|---|---|
| L-aspartate dimethyl ester, HCl | 19 | >95 |
| L-histidine methyl ester, HCl | 18 | >95 |
| L-glutaminic acid methyl ester, HCl | 19 | >95 |
| L-lysine methyl ester, HCl | 19 | >95 |

### Example 12

### Preparation of 28-Aspartateamide Dimethyl Ester of Betulonic Acid

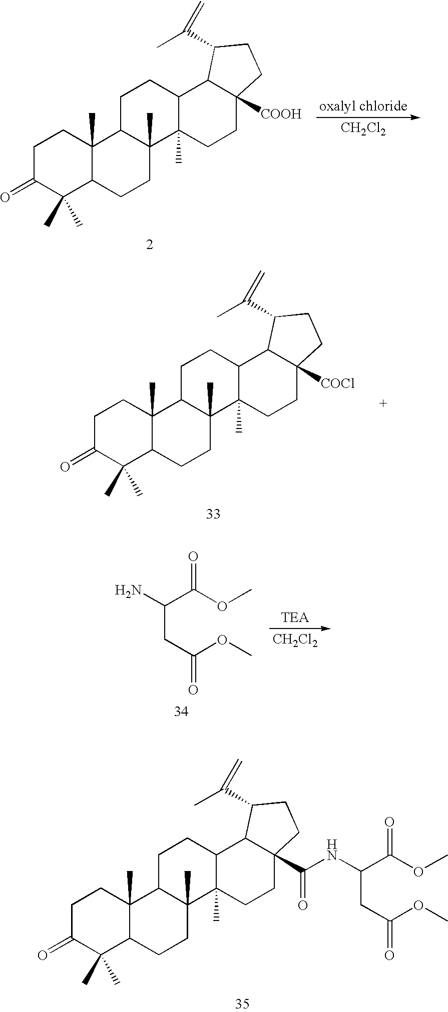

Betulonic acid 2 (8.8 mmol) was dissolved in dichloromethane under inert atmosphere, followed by the addition of oxalyl chloride (18.6 mmol). The reaction mixture was agitated at room temperature for 20 hours. After completion of the reaction, the solvent was evaporated to dryness, the residue was again dissolved in dichloromethane, which was once more evaporated to dryness. The raw product obtained was washed with diethyl ether. The yield was 7.5 mmol (85%) of betulonic acid chloride 33. Betulonic acid chloride 33 (4.2 mmol) and L-aspartic acid dimethyl ester hydrochloride 34 (5.5 mmol) were dissolved in dichloromethane, and triethyl amine (11 mmol) was added. The reaction mixture was agitated at room temperature for 20 hours. The reaction mixture was washed with diluted hydrochloric acid solution, water and dried over sodium sulfate. The solvent was evaporated to dryness, followed by purification of the raw product by chromatography, if necessary. Yield was 1.8 mmol (43%) of the 28-aspartateamide dimethyl ester of betulonic acid 35.

### Example 13

### Preparation of 28-N-Acetylanthranilic Acid Ester of Betulin

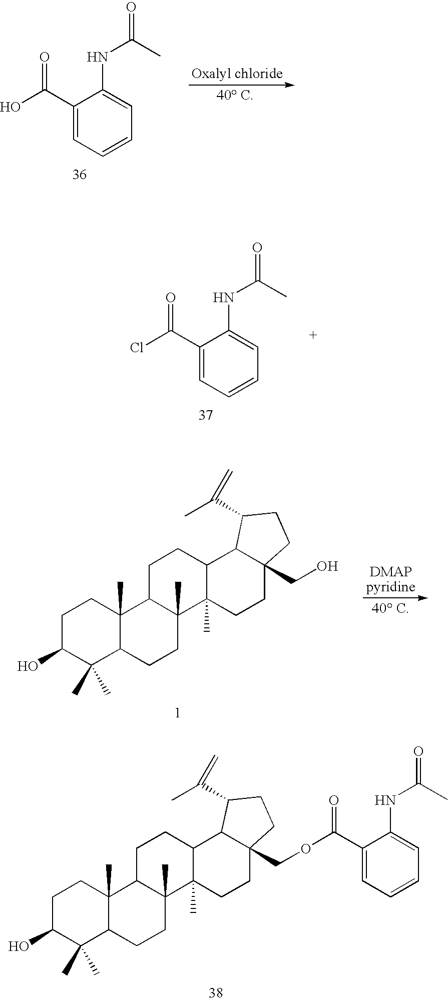

A mixture of N-acetylanthranilic acid 36 (25.0 mmol) and oxalyl chloride (250 mmol) was mixed for 16 hours at 40° C. Excessive oxalyl chloride was removed by evaporating the reaction mixture to dryness. The residue was twice dissolved in dichloromethane, which was evaporated to dryness, thus giving N-acetylanthranilic acid chloride 37 with a quantitative yield. A mixture of betulin 1 (11.29 mmol), DMAP (11.29 mmol), N-acetylanthranilic acid chloride 37 and pyridine (80 ml) was agitated for 24 hours at 40° C. After completion of the reaction, the reaction mixture was diluted with ethyl acetate and washed with dilute hydrochloric acid solution, and water and dried over sodium sulfate. The solvent was evaporated, followed by purification of the raw product by chromatography, thus giving 28-N-acetylanthranilic acid ester of betulin 38 (yield: 25%).

### Example 14

### Preparation of 28-Nicotinic Acid Ester of Betulin (Comparative)

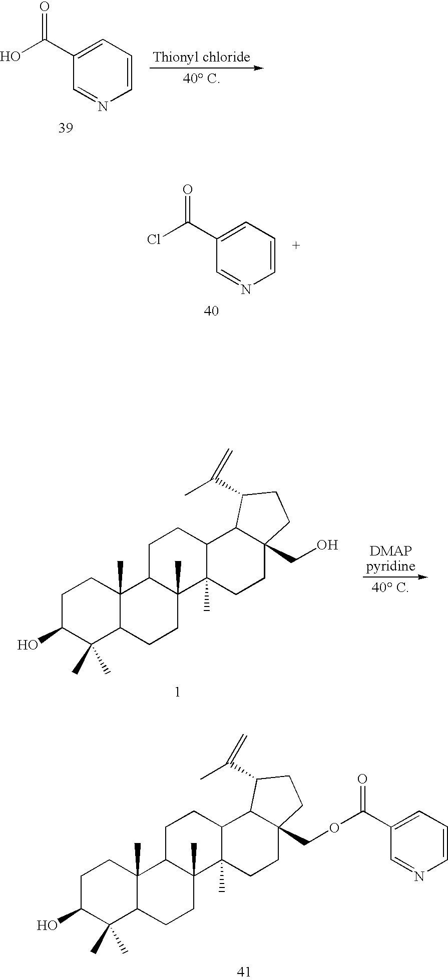

A mixture of nicotinic acid 39 (25.0 mmol) and thionyl chloride (250 mmol) was mixed for 24 hours at 40° C. Excessive thionyl chloride was removed by evaporating the reaction mixture to dryness. The residue was twice dissolved in dichloromethane, which was evaporated to dryness, thus yielding nicotinic acid chloride 40. A mixture of betulin 1 (2.26 mmol), DMAP (2.26 mmol), nicotinic acid chloride 40 (2.71 mmol) and pyridine (10 ml) was agitated for 24 hours at 40° C. After completion of the reaction, the reaction mixture was diluted with ethyl acetate and washed with dilute hydrochloric acid solution, and water and dried over sodium sulfate. The solvent was evaporated, followed by purification of the raw product by recrystallization in cyclohexane, thus giving 28-nicotinic acid ester of betulin 41 with a yield of 88%.

### Example 15

### Preparation of 3,28-diacetoxy-19,20-ene-29-succinic anhydride of betulin

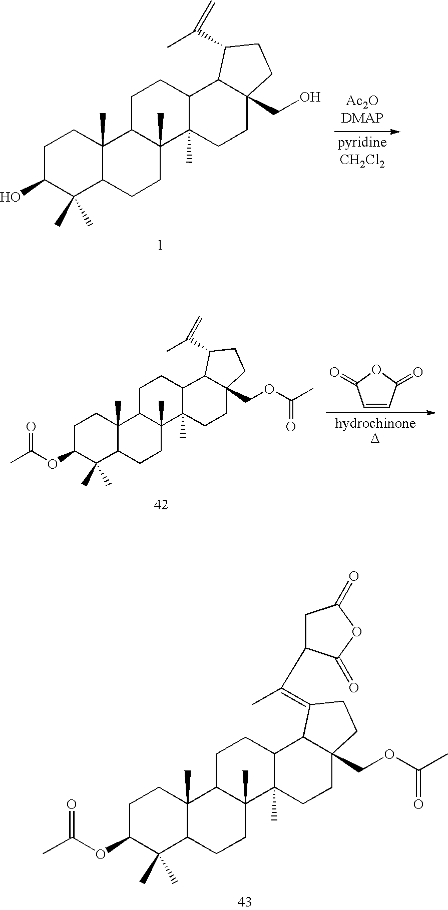

a) Acetic anhydride (19.2 ml, 203 mmol) was added to a mixture of betulin 1 (15.0 g, 33.88 mmol), DMAP (0.41 g, 3.39 mmol), pyridine (25 ml, 309 mmol), and dichloromethane (150 ml). The reaction mixture was agitated at room temperature for 17 hours. The organic phase was washed with 10% hydrochloric acid solution (200 ml), saturated NaHCO₃ solution (400 ml), water (100 ml), and dried over Na₂SO₄. The solvent was evaporated in vacuum, thus giving 3,28-diacetoxy betulin 42 (yield: 97%).
b) A mixture of 3,28-diacetoxy betulin 42 (4.57 g, 8.68 mmol) and hydrochinone (96 mg, 0.87 mmol) was heated at 200° C., followed by the addition of succinic anhydride (2.50 g, 25.02 mmol) during 2 hours to the reaction flask. After completion of the reaction, the raw product, 3,28-diacetoxy-19,20-ene-29-succinic anhydride of betulin 43 was obtained with a yield of 100% (5.41 g, 8.65 mmol).

### Example 16

### Preparation of 3-deoxy-2,3-dihydrobetulin (comparative)

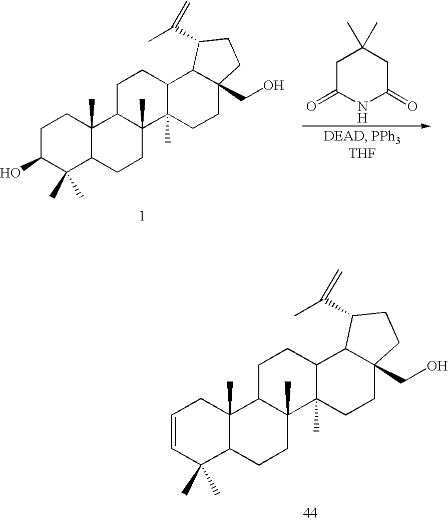

A solution of diethylazo dicarboxylate (DEAE, 20.71 ml, 45.18 mmol) was added dropwise under a nitrogen atmosphere to a mixture of betulin 1 (5.00 g, 11.29 mmol), triphenyl phosphine (PPh₃, 11.85 g, 45.18 mmol), and 3,3-dimethyl glutarimide (6.38 g, 45.18 mmol) in dry THF (100 ml) in an ice bath. The reaction mixture was allowed to warm to room temperature, and agitating was continued for 24 hours. The precipitate formed was separated by filtering, followed by evaporating the solvent in vacuum. The raw product was purified by chromatography, thus giving 3-deoxy-2,3-dihydrobetulin 44 (1.47 g, 3.45 mmol, 31%).

### Example 17

### Preparation of 3-O-Diels-Alder adduct of betulin

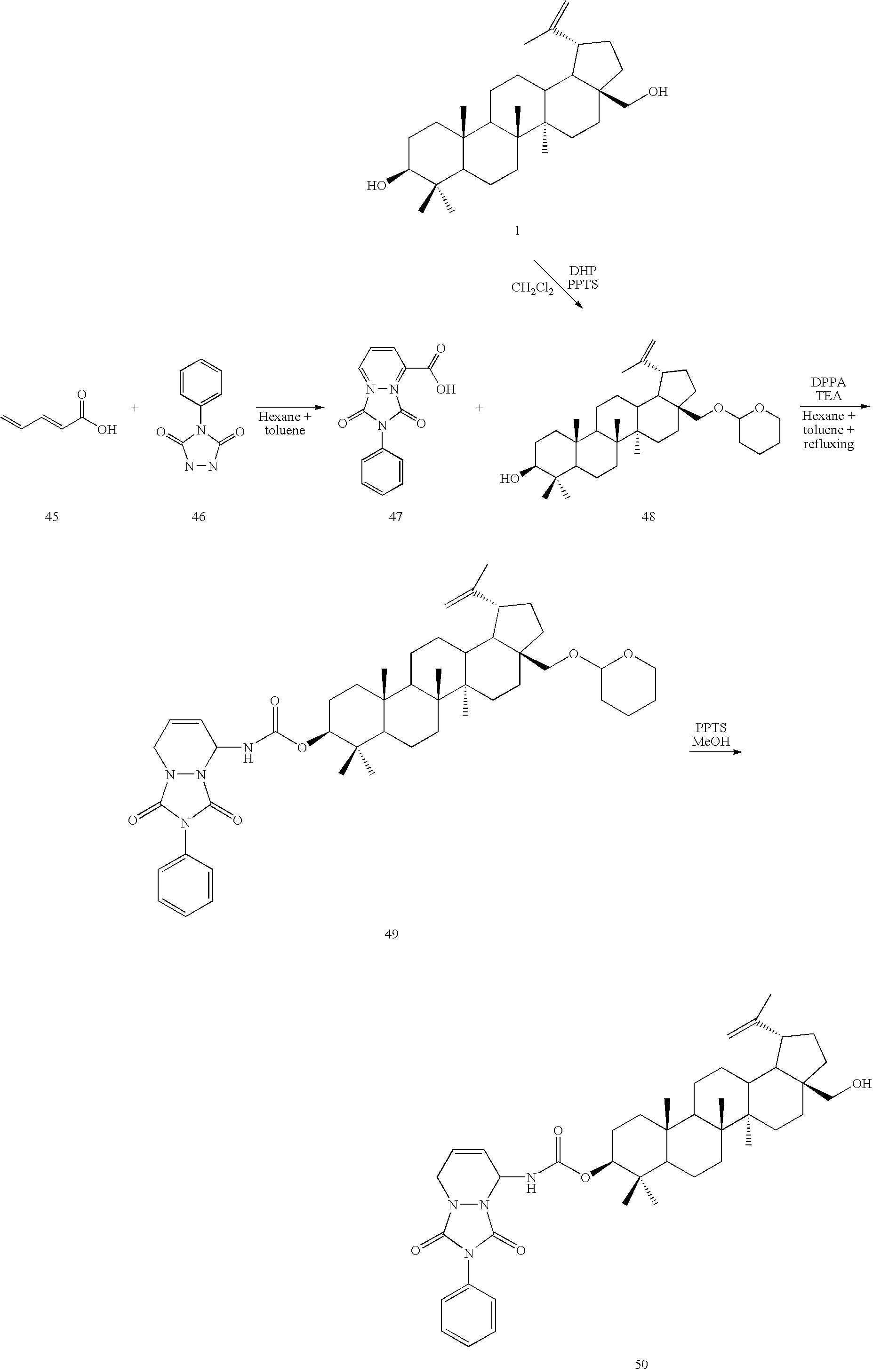

2,4-pentadiene acid 45 (196 mg, 2.0 mmol) and 4-phenyl-1,2,4-triazolin-3,5-dion 46 (350 mg, 2.0 mmol) were dissolved in a mixture of hexane and toluene. The reaction mixture was agitated under inert atmosphere at room temperature for 3 days. After completion of the reaction, the solvent was evaporated, thus giving the Diels-Alder adduct 47 (493 mg, 1.80 mmol, 90%).

Pyridinium-p-toluenesulfonate (PPTS) (0.68 g, 2.71 mmol) and dihydropyran (DHP) (2.09 g, 24.9 mmol) were added in betulin 1 (10.0 g, 22.6 mmol) in dichloromethane (330 ml) under inert atmospere, and then the reaction mixture was agitated at room temperature for 5 days. After completion of the reaction, the organic phase was washed with saturated NaHCO₃ solution (150 ml) and water (150 ml), followed by drying over Na₂SO₄. The solvent was evaporated in vacuum, and the raw product obtained was purified by chromatography, thus giving the 28-tetrahydropyran ether of betulin 48 (3.46 g, 6.55 mmol, 29%).

28-tetrahydropyran ether of betulin 48 (116 mg, 0.22 mmol) and the Diels-Alder adduct 47 (60 mg, 0.22 mmol) were dissolved in a mixture of hexane and toluene. Diphenylphosphoryl azide (DPPA) and triethylamine (TEA) were added to the reaction mixture, which was refluxed for 24 hours. After completion of the reaction, the reaction mixture was diluted with ethyl acetate, the organic phase was washed with water, NaHCO₃ solution, diluted hydrochloric acid solution and water, followed by drying over Na₂SO₄. The solvent was evaporated in vacuum, thus giving raw product (419 mg) that was purified by chromatography, thus giving the 3-O-Diels-Alder adduct of the 28-tetrahydropyran ether of betulin 49 with a yield of 50%.

A mixture of the 3-O-Diels-Alder adduct of the 28-tetrahydropyran ether of betulin 49 (50 mg, 0.063 mmol), pyridinium-p-toluene sulfonate (PPTS) (3 mg, 0.013 mmol), and methanol (10 ml) was agitated at room temperature under an inert atmosphere for two weeks. After completion of the reaction, NaHCO₃ solution (10 ml) was added to the reaction mixture. The aqueous phase was extracted with ethyl acetate (40 ml), which was washed with water (80 ml), dried over Na₂SO₄, followed by evaporation of the solvent in vacuum. The raw product was purified by chromatography. 3-O-Diels-Alder adduct of betulin 50 was thus obtained with a yield of 50%.

### Example 18

### Preparation of the 4-methylurazole-Diels-Alder-adduct of betulin

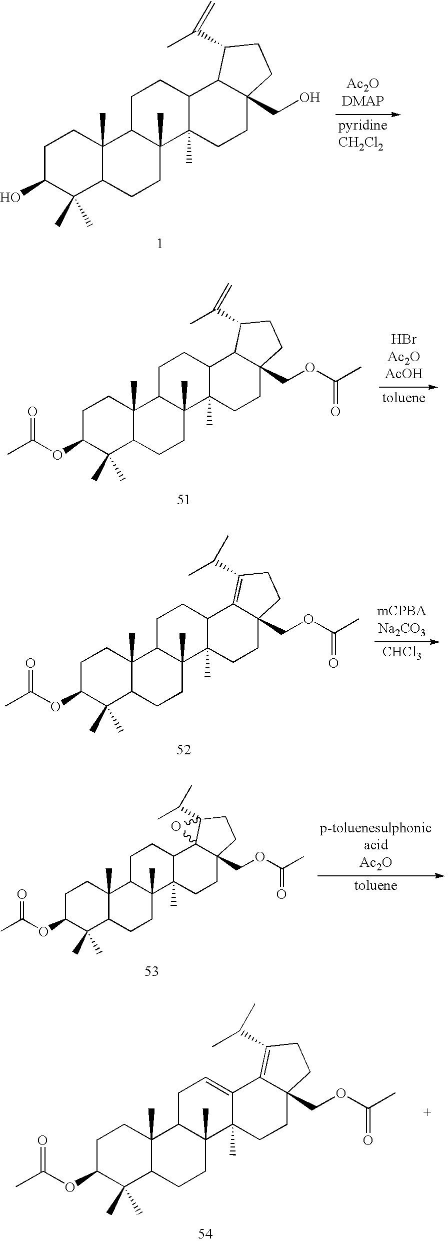

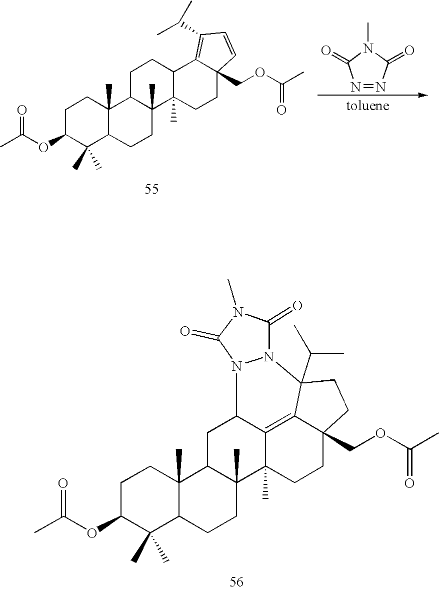

To a mixture of betulin 1 (15.0 g, 33.88 mmol), N,N-dimethylamino pyridine (DMAP, 0.41 g, 3.39 mmol), pyridine (25 ml, 309 mmol), and dichloromethane (150 ml), acetic anhydride (19.2 ml, 203 mmol) was added. The reaction mixture was mixed at room temperature for 17 hours. Organic phase was washed with 10% hydrochloric acid solution (200 ml), saturated NaHCO₃ solution (400 ml), and water (100 ml) and dried over Na₂SO₄. The solvent was evaporated in vacuum, thus giving betulin 3,28-diacetate 51 (yield: 97%).

To a mixture of hydrobromic acid (HBr) (47%, 250 g), acetic anhydride (100 g), and acetic acid (300 g), betulin 3,28-diacetate 51 (17.41 g, 33.05 mmol) dissolved in toluene (200 ml) was added. The reaction mixture was allowed to stand at room temperature for three weeks. The reaction mixture was diluted with water (400 ml). The aqueous phase was separated and extracted with toluene (400 ml). Pooled organic phases were washed with water (30 ml), saturated NaHCO₃ solution (600 ml), dried over Na₂SO₄, and the solvent was evaporated in vacuum. The raw product was purified by chromatography, thus giving 3β,28-diacetoxylup-18-ene 52 (7.36 g, 13.97 mmol, 42%).

To a mixture of 3β,28-diacetoxylup-18-ene 52 (4.91 g, 9.33 mmol) and Na₂CO₃ (4.94 g, 46.65 mmol) in chloroform (120 ml), m-chloroperbenzoic acid (mCPBA, 3.69 g, 14.92 mmol) was added, followed by agitation of the reaction mixture at room temperature for two hours. The organic phase was washed with water (150 ml), saturated NaHSO₃ solution (150 ml), saturated NaHCO₃ solution (150 ml), dried over Na₂SO₄, and the solvent was evaporated in vacuum. The raw product was recrystallized in ethanol, thus giving 3β,28-diacetoxylup-18,19-epoxylupane 53 (3.31 g, 6.09 mmol, 65%).

3β,28-diacetoxylup-18,19-epoxylupane 53 (2.00 g, 3.68 mmol) and p-toluenesulfonic acid (0.42 g, 2.21 mmol) were dissolved in toluene (80 ml), and then acetic anhydride (0.56 ml, 5.90 mmol) was added. The reaction mixture was refluxed for four hours. Organic phase was washed with saturated NaHCO₃ solution (150 ml), and water (100 ml), dried over Na₂SO₄, and the solvent was evaporated in vacuum. The raw product was purified by chromatography and crystallized in ethanol, thus giving a mixture of 3β,28-diacetoxylupa-12,18-diene 54 and 3β,28-diacetoxylupa-18,21-diene 55 (4:1) (1.31 g, 2.50 mmol, 68%).

3β,28-diacetoxylupa-12,18-diene 54, 3β,28-diacetoxylupa-18,21-diene 55 (total amount of 100 mg, 0.19 mmol), and 4-methyl-1,2,4-triazolin-3,5-dion (32 mg, 0.29 mmol) were dissolved in toluene (5 ml), and then the reaction mixture was agitated at room temperature for 24 hours. The solvent was evaporated in vacuum and the raw product was purified by chromatography, thus giving Diels-Alder-adduct of 4-methylurazole with betulin 56 (60 mg, 0.09 mmol, 49%).

### Example 19

### Preparation of Diels-Alder adduct of p-acetyl-4-phenylurazole with betulin

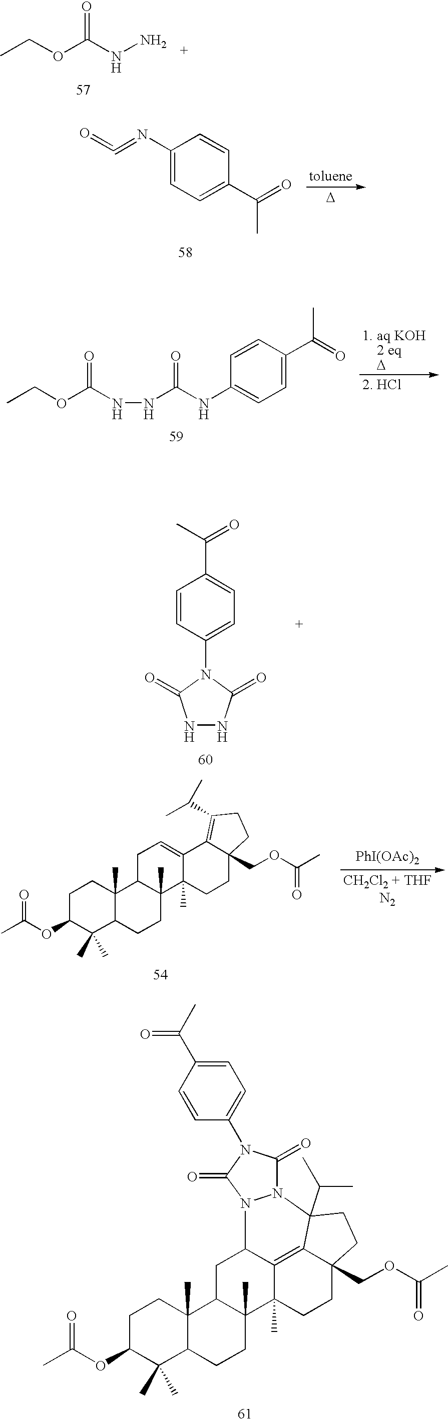

To ethylhydrazin 57 (2.64 mmol) in toluene (5 ml), 4-acetylphenyl isocyanate 58 (2.64 mmol) dissolved in 5 ml of toluene was added dropwise under an inert atmosphere. Agitation was continued for 2 hours at room temperature, and at 80° C. for 2 hours. Filtering of the precipitate formed and drying thereof in the oven gave p-acetyl-4-phenyl-1-carbethoxy semicarbazide 59 (yield: 90%).

This p-acetyl-4-phenyl-1-carbethoxy semicarbazide 59 (1.13 mmol) was heated at 70° C. in an aqueous 4M KOH solution (2.26 mmol) for 1.5 hours. The precipitate was filtered off, followed by acidification of the cooled filtrate with concentrated HCl solution. The precipitate formed was filtered and dried in a desiccator, thus giving p-acetyl-4-phenylurazole 60 (yield: 65%).

A mixture of p-acetyl-4-phenylurazole 60 (50 mg, 0.229 mmol), and iodobenzene diacetate ((PhI(OAc)₂, 74 mg, 0.229 mmol) was agitated under Ar gas in an anhydrous THF:CH₂Cl₂ mixture (4 ml, 1:1) for 15 minutes yielding a red colour. 3β,28-diacetoxylupa-12,18-diene 54 (100 mg, 0.191 mmol) was dissolved in a THF:CH₂Cl₂ mixture (4 ml, 1:1) and added to the reaction flask, and agitation was continued for 24 hours at room temperature. The solvent was evaporated in vacuum. Purification of the raw product by chromatography gave a Diels-Alder adduct of betulin with p-acetyl-4-phenylurazole 61 with a yield of 30%. Table 3 below shows the percent yields of the Diels-Alder adducts of betulin with urazole for different groups R:

TABLE 3

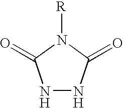

| R | Yield (%) |
|---|---|
| 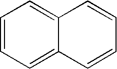 | 53 |
| 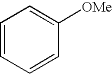 | 47 |
| H | 40 |
| 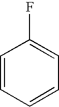 | 44 |
| 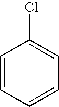 | 74 |
| 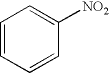 | 60 |
| 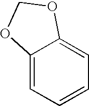 | 51 |
| 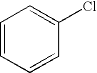 | 38 |
| 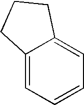 | 53 |
| 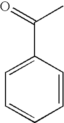 | 30 |
| 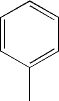 | 62 |

### Example 20

### Preparation of betulin 3-acetoxy-28-1′,2′,3′-triazoles and betulin 3-acetoxy-28-tetrazoles

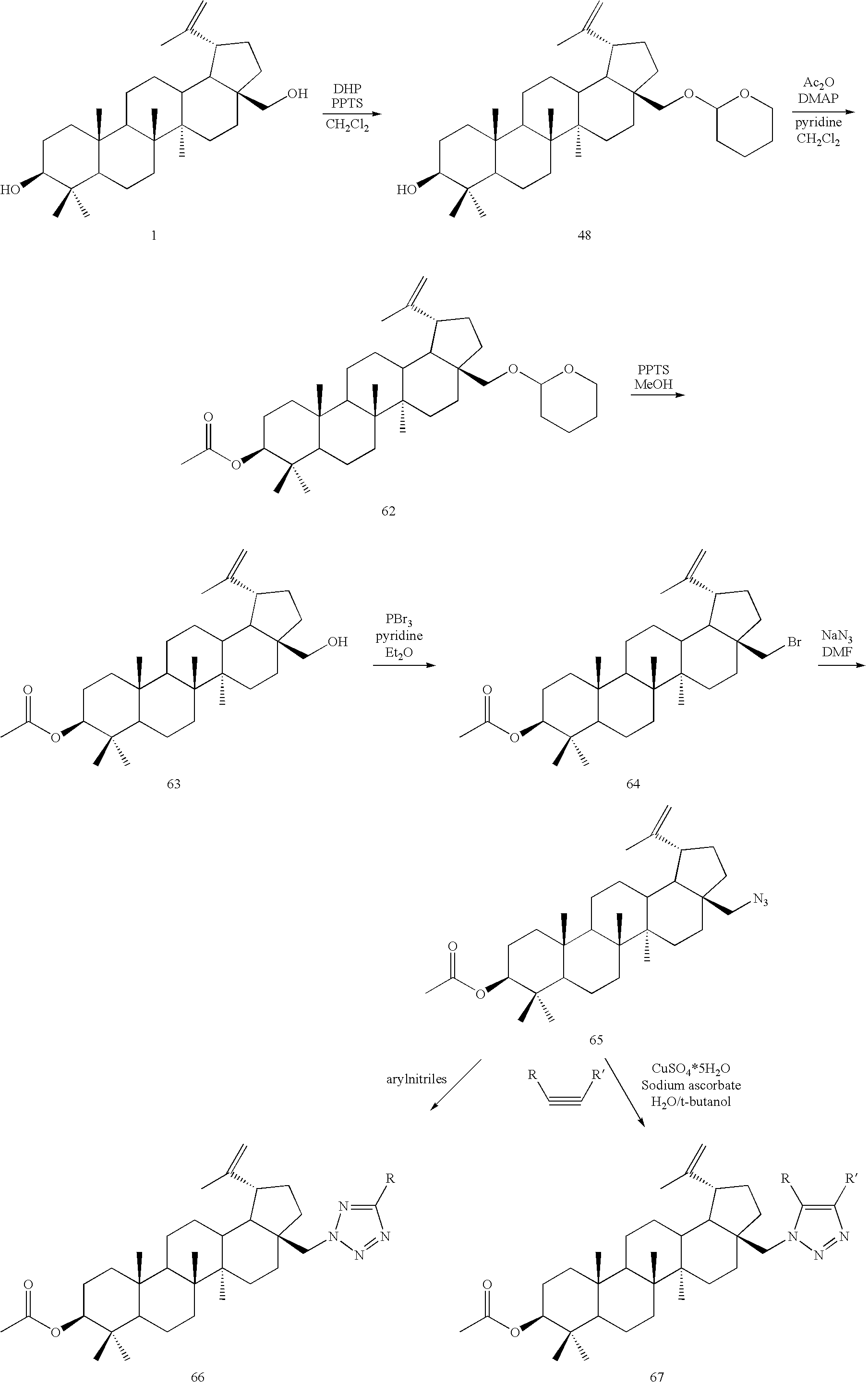

To betulin 1 (10.0 g, 22.6 mmol) in dichloromethane (330 ml), pyridinium-p-toluenesulfonate (PPTS) (0.68 g, 2.71 mmol), and dihydropyrane (DHP) (2.09 g, 24.9 mmol) were added under inert atmosphere, followed by agitation of the reaction mixture at room temperature for 5 days. After completion of the reaction, the organic phase was washed with saturated NaHCO₃ solution (150 ml) and water (150 ml), then dried over Na₂SO₄. The solvent was evaporated in vacuum, and then the raw product was purified by chromatography, thus giving betulin 28-tetrahydropyrane ether 48 (3.46 g, 6.55 mmol, 29%).

To a mixture of betulin 28-tetrahydropyrane ether 48 (5.00 g, 9.49 mmol), N,N-dimethylamino pyridine (DMAP, 0.12 g, 0.95 mmol), pyridine (10 ml, 124 mmol), and dichloromethane (50 ml), acetic anhydride (5.4 ml, 57 mmol) was added. The reaction mixture was agitated at room temperature for 20 hours. The organic phase was washed with 10% hydrochloric acid solution (300 ml), saturated NaHCO₃ solution (400 ml), water (100 ml), and dried over Na₂SO₄. The solvent was evaporated in vacuum, thus giving betulin 3-acetoxy-28-tetrahydropyrane ether 62 (yield: 95%).

A mixture of betulin 3-acetoxy-28-tetrahydropyrane ether 62 (3.00 g, 5.27 mmol), pyridinium-p-toluenesulfonate (PPTS) (226 mg, 1.06 mmol), and methanol (100 ml) was agitated at room temperature under an inert atmosphere for 2 weeks. After completion of the reaction, NaHCO₃ solution (100 ml) was added to the reaction mixture. The aqueous phase was extracted with ethyl acetate (400 ml), followed by washing with water (800 ml), dried over Na₂SO₄, the solvent was evaporated in vacuum, thus giving betulin 3-acetate 63 (yield: 94%).

To a mixture of betulin 3-acetate 63 (100 mg, 0.21 mmol) and diethyl ether (10 ml), pyridine (163 mg, 2.1 mmol) and phosphorus tribromide (PBr₃) (280 mg, 1.0 mmol) were added at −5° C. under an inert atmosphere. The reaction mixture was allowed to warm to room temperature while continuing mixing for 24 hours. After completion of the reaction, the organic phase was washed with water (100 ml), NaHCO₃ solution (80 ml) and dried over Na₂SO₄. The solvent was evaporated in vacuum, thus giving betulin 3-acetoxy-28-bromide 64 (yield: 63%).

A mixture of betulin 3-acetoxy-28-bromide 64 (200 mg, 0.36 mmol), NaN₃ (230 mg, 3.6 mmol), and DMF (20 ml) was heated at 100° C. under an inert atmosphere for 24 hours. After completion of the reaction, the solvent was evaporated in vacuum and the residue was taken up in ethyl acetate (100 ml). The organic phase was washed with water (225 ml), dried over Na₂SO₄ and the solvent was evaporated in vacuum, thus giving 149 mg of the raw product comprising 20% of betulin 3-acetoxy-28-azide 65.

Using known methods, betulin 3-acetoxy-28-azide 65 may be reacted with arylnitriles, giving betulin 3-acetoxy-28-tetrazoles 66, or with a functional alkyne in the presence of CuSO₄±5H₂O and sodium ascorbate in an aqueous butanol solution, giving betulin 3-acetoxy-28-1′,2′,3′-triazoles 67.

### Example 21

### Preparation of betulin 3,28-dibetaine ester

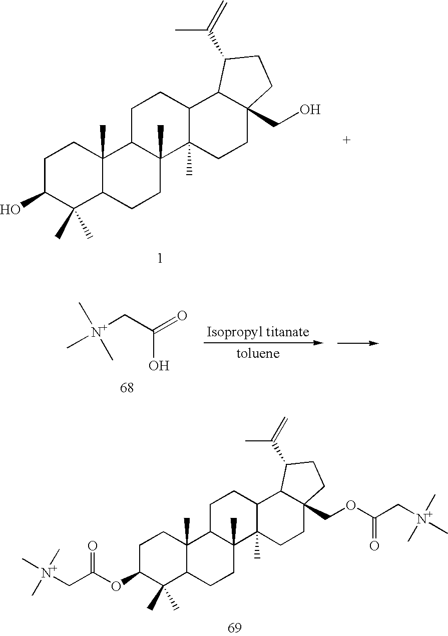

Betulin 1 (7.0 g, 16 mmol) and betaine 68 (3.8 g, 32 mmol) were dissolved in toluene (150 ml) while heating. Thereafter, isopropyl titanate Ti(OCHMe₂)₄ catalyst (0.85 g, 3 mmol) was added, and the mixture was refluxed for 3 hours. The solid final product was separated by filtration. Tetrahydrofurane was added to remove by-products, and filtering was repeated. Yield of the final product 69 (betulin 3,28-dibetaine ester) was 2.7 g (4.1 mmol, 26%).

### Example 22

### Preparation of 28-acetate of betulonic alcohol

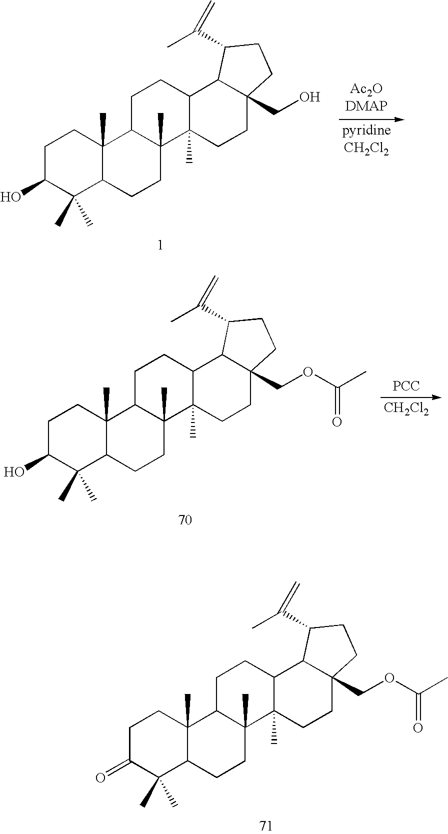

a) To a mixture of betulin 1 (8.00 g, 18.1 mmol) and 4-dimethylamino pyridine (DMAP) (0.8 g, 6.55 mmol) in dichloromethane (72 ml), pyridine (72 m) and acetic anhydride (1.8 ml, 19.1 mmol) were added, and the reaction mixture was agitated at room temperature for 22 hours. The organic layer was washed with 10% hydrochloric acid solution, water, saturated NaHCO₃ solution, and dried over Na₂SO₄. The solvent was evaporated in vacuum, followed by purification of the raw product obtained by chromatography, thus giving 28-acetoxybetulin 70 (3.80 g, 45%).
b) A mixture of betulin 28-acetate (590 mg, 1.23 mmol) and pyridinium chlorochromate (PCC) (1.32 g, 3.14 mmol) in dichloromethane (60 ml) was agitated at room temperature for 24 hours. The reaction mixture was diluted with diethyl ether (30 ml), agitated for 10 minutes, and the precipitate was filtered off. The filtrate was evaporated in vacuum and the raw product was purified by chromatography, thus giving 28-acetate of betulonic alcohol 71 (330 mg, 57%).

### Example 23

### Preparation of Betulonic and Betulinic Acids (Comparative)

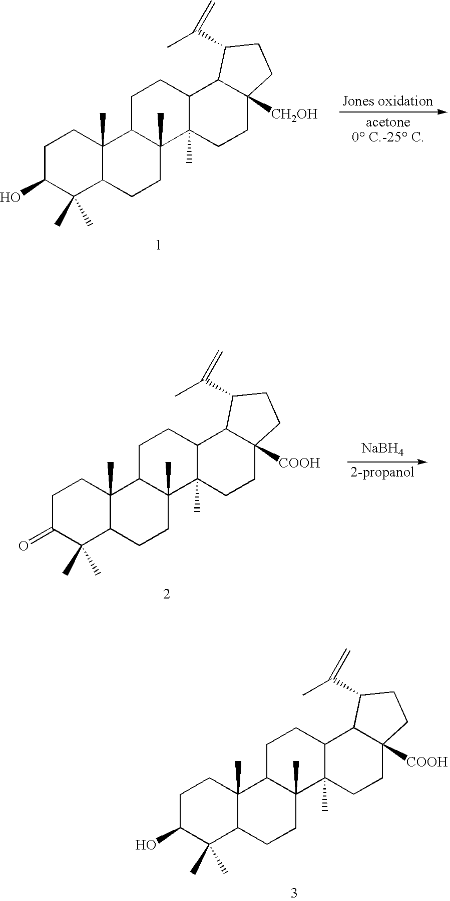

a) To a solution of betulin 1 (50 g, 113 mmol) in acetone (1500 ml), a Jones reagent was added during 1 hour in an ice bath. The reaction mixture was allowed to warm to room temperature, and agitation was continued for 21 hours. Methanol (700 ml) and water (1000 ml) were added to the reaction mixture. The precipitate was filtered, dried in vacuum, taken up in diethyl ether (600 ml) and washed with water, 7.5% hydrochloric acid, water, saturated NaHCO₃ solution, and water. Half of the diethyl ether was evaporated in vacuum and the residue was treated with 10% NaOH solution. The precipitate was filtered, dried in vacuum, and dissolved in boiling methanol, followed by the addition of acetic acid (10 ml) thereto. The product was precipitated with water, filtered and dried in vacuum, thus giving betulonic acid 2 (22.3 g, 44%).
b) To betulonic acid 2 (10 g, 22 mmol) in 2-propanol (400 ml), NaBH₄ (1.76 g, 44.2 mmol) was added, and the reaction mixture was agitated at room temperature for 2 hours. 10% hydrochloric acid solution (600 ml) was added, the precipitate was filtered, washed with water and dried in vacuum. The raw product obtained was crystallized in ethanol, thus giving betulinic acid 3 (8.25 g, 18 mmol).

### Example 24

### Preparation of Betulonic Aldehyde (Comparative)

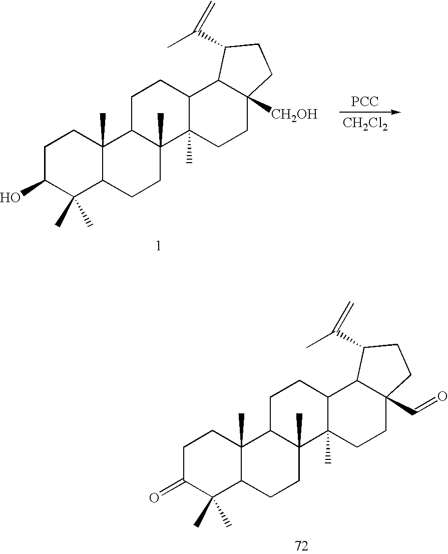

A mixture of betulin 1 (3.0 g, 6,8 mmol), pyridinium chlorochromate (PCC) (8.8 g, 41 mmol) and dichloromethane was agitated at room temperature for 1 hour. The reaction mixture was dissolved with diethyl ether and filtered through alumina. The filtrate was washed with water, 5% hydrochloric acid, again with water and dried over Na₂SO₄. The solvent was evaporated in vacuum and the raw product was crystallized in a mixture of hexane and ethyl acetate, thus giving betulonic aldehyde 72 (2.4 g, 82%).

### Example 25

### Preparation of 28-methyl ester of betulinic acid

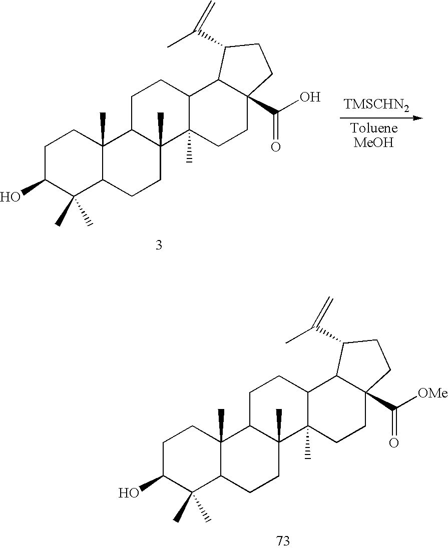

To a mixture of betulinic acid 3 (100 mg, 0.22 mmol), methanol (1 ml) and toluene (1.5 ml), a 2M solution of trimethylsilyl diazomethane in diethyl ether (0.17 ml, 0.33 ml) was added and the reaction mixture was agitated at room temperature for 40 minutes. The solvent was evaporated in vacuum, thus giving 28-methyl ester of betulinic acid 73 (68 mg, 66%).

### Example 26

### Preparation of betulin aldehyde, betulin 28-oxime and betulin 3,28-dioxime

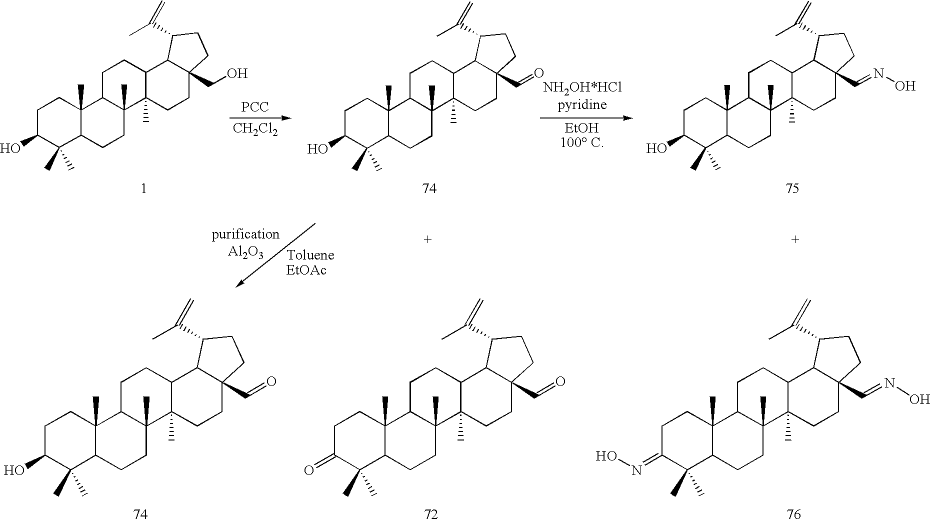

a) A mixture of betulin 1 (8.0 g, 18 mmol) and pyridinium chlorochromate (PCC) (7.0 g, 33 mmol) in dichloromethane (800 ml) was agitated at room temperature for 40 minutes. The reaction mixture was diluted with diethyl ether (200 ml) and filtered through alumina. The solvent was evaporated in vacuum and the raw product was purified by chromatography, thus giving betulin aldehyde 74 (0.36 g, 18%).
b) To a mixture of betulonic aldehyde 72, betulinic aldehyde 74, pyridine (40 ml) and ethanol (120 ml), hydroxylamine hydrochloride (10 g, 144 mmol) was added, followed by refluxing the reaction mixture for 18 hours. The solvent was evaporated in vacuum, treated with water and filtered. The precipitate was dried in desiccator and the mixture of betulin 28-oxime 75 and betulin 3,28-dioxime 76 obtained was purified by chromatography, thus giving betulin 28-oxime 75 (0.97 g, 2.1 mmol) and betulin 3,28-dioxime 76 (0.32 g, 0.7 mmol).

### Example 27

### Preparation of Betulonic Alcohol

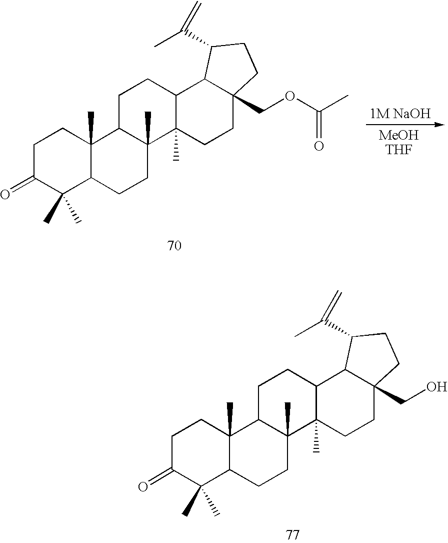

A mixture of betulonic 28-acetate 70 (15 mg, 0.032 mmol), methanol (0.3 ml), tetrahydrofurane (0.45 ml) and 1 M NaOH solution (0.16 ml) was agitated at room temperature for 20 hours. Water (4 ml) was added and the reaction mixture was made acidic with dilute hydrochloric acid. The aqueous phase was extracted with ethyl acetate, which was dried over Na₂SO₄ and evaporated in vacuum, thus giving 77 (7.0, 50%).

### Example 28

### Preparation of betulin 3-acetoxyoxime-28-nitrile

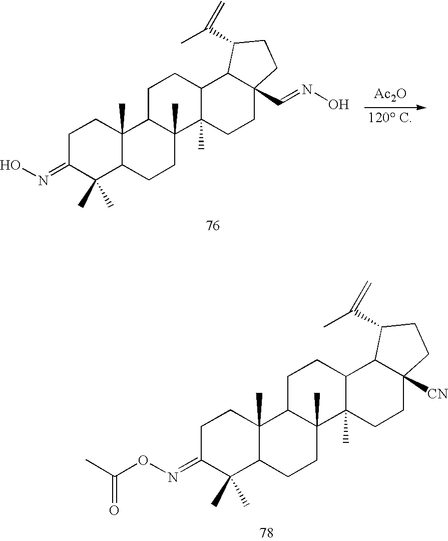

A mixture of betulin 3,28 dioxime 76 (100 mg, 0.2 mmol) and acetic anhydride (2.5 ml) was agitated at 120° C. for 2 hours. The reaction mixture was diluted with water and the precipitate was filtered off. The precipitate was taken up in chloroform, washed with water, saturated NaHCO₃ solution, water and dried over Na₂SO₄. The solvent was evaporated in vacuum and the raw product was purified by chromatography, thus giving betulin 3-acetoxyoxime-28-nitrile 78 (37 mg, 34%).

### Example 29

### Preparation of betulin 28-acetic acid methyl ester

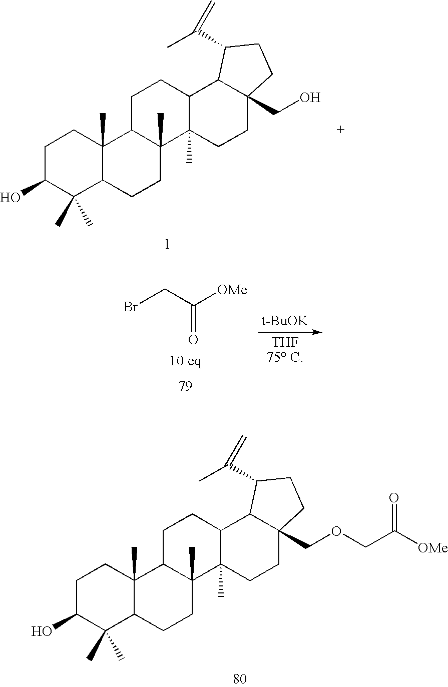

A mixture of betulin 1 (1.0 g, 2.3 mmol) and potassium tert-butoxide (2.5 g, 23 mmol) in tetrahydrofurane (50 ml) was agitated at 75° C., followed by the addition of methylbromoacetate 79 (2.1 ml, 23 mmol). The reaction mixture was agitated for 10 minutes, allowed to cool and then diluted with water. The precipitate was filtered and the raw product was purified by chromatography, thus giving betulin 28-acetic acid methyl ester 80 (0.2 g, 15%).

### Example 30

### Preparation of 20,29-dihydrobetulin and 20,29-dihydrobetulonic acid

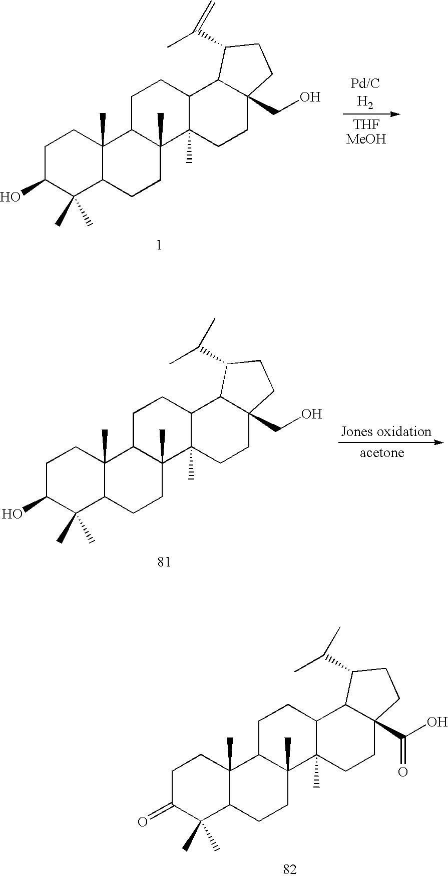

a) To a mixture of betulin 1 (2.0 g, 4.5 mmol), tetrahydrofurane (40 ml) and methanol (80 ml), 5% Pd/C (0.2 g) was added, followed by agitating the reaction mixture under hydrogen atmosphere for 22 hours. The reaction mixture was filtered, and the filtrate was evaporated in vacuum, thus giving 20,29-dihydrobetulin 81(2.0 g, 99%).
b) To a mixture of 20,29-dihydrobetulin 81 (1.0 g, 2.3 mmol) and acetone (75 ml), Jones reagent was added. The reaction mixture was agitated for 20 hours. Methanol (20 ml) and water (40 ml) were added to the reaction mixture. The organic solvent was evaporated in vacuum and the aqueous phase was extracted with ethyl acetate, which was washed with water and dried over Na₂SO₄. The solvent was evaporated in vacuum and the raw product was purified by chromatography, thus giving 20,29-dihydrobetulonic acid 82 (320 mg, 31%).

### Example 31

### Preparation of a Diels-Alder adduct of 4-methylurazole

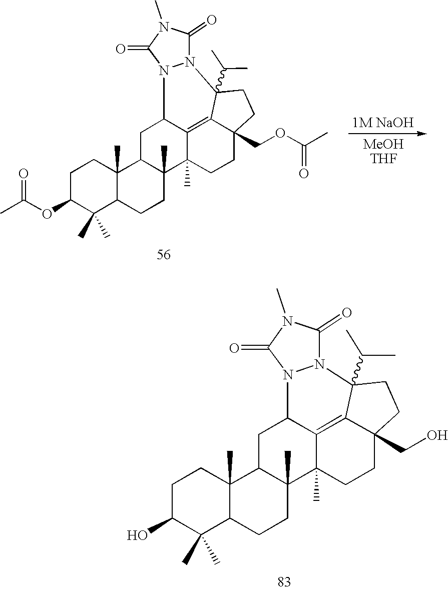

A mixture of Diels-Alder adduct of 4-methylurazole 56 (50 mg, 0.07 mmol), methanol (0.5 ml), tetrahydrofurane (0.8 ml) and 1 M aqueous NaOH solution (0.3 ml) was agitated at room temperature for 20 hours. The product was precipitated with water, the precipitate was filtered and dried, thus giving the Diels-Alder adduct of 4-methylurazole 83 (40 mg, 91%).

### Example 32

### Cytotoxicity Tests of the Betulin Derived Compounds

Caco-2 cells (cell line used as a model for human intestine) were introduced in a 96 well plate in an amount of 35 000 cells (for LDH method), 45 000 cells (for WST-1 method), or 25 000 cells (for ATP method) per well. After cultivation of 24 hours, the cells were exposed to the compounds being tested for 24 hours by adding said compounds to the culture medium to give a concentration of 500 μM (as stock solutions in DMSO).

The influence of the compounds on the viability of the cells was measured by three different methods. Polymyxin B was used as the control. Lactate dehydrogenase (LDH) is an enzyme found in cells, and accordingly, increased amounts thereof outside cells result from cell membrane damage. The amount of LDH in the sample due to exposure was quantified by means of an enzymatic reaction using the INT (iodonitrotetrazolium) colour reagent wherein the coloured reaction product formed was determined photometrically at 490 nm. In the WST-1 method, the metabolic activity of the cells after exposure was measured using the WST-1 reagent. Metabolic activity of a cell results in the generation of a coloured product from the reagent, said product being then used to evaluate the viability of the cells by photometric measurements (absorbance at 440 nm). In the ATP method, the amount of ATP within cells decreasing rapidly due to cellular damage was measured. In the method, ATP was luminometrically quantified by means of the ATP dependent luciferase-luciferin reaction.

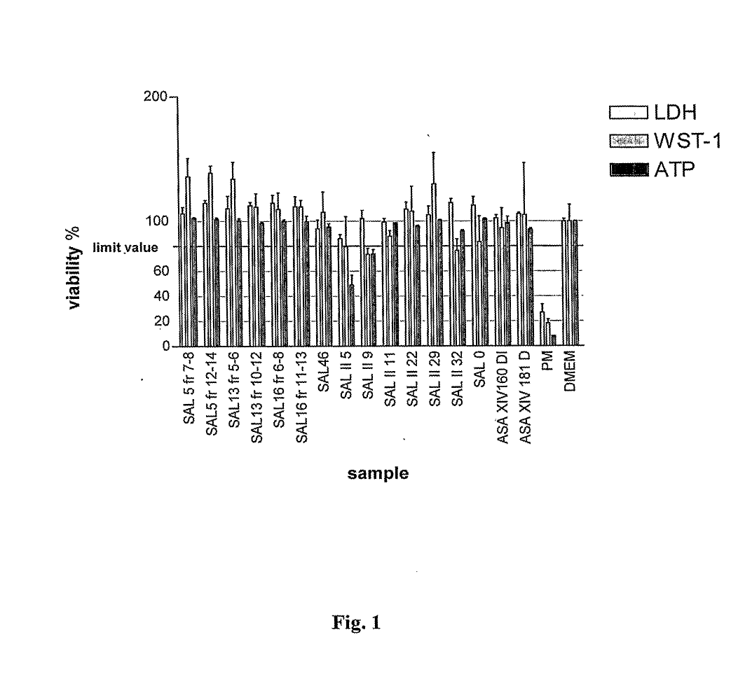

Appended FIG. 1 shows effects on the viability of Caco-2 cells (%) after exposure for 24 hour as measured by three methods for the determination of cellular viability (LDH, WSR-1 and ATP methods). Compounds exceeding the limit value, i.e. 80% viability, are considered to have no significant negative effect on the viability of cells in vitro. The compounds of the Table 4 below were used for testing.

TABLE 4

| Code | Compound |
|---|---|
| PM | positive control (polymyxin B sulfate) |
| Sal-5 fr. 7-8 | 3,28-O-isostearylic acid diester of betulin |
| Sal-5 fr. 12-14 | 28-O-isostearylic acid ester of betulin |
| Sal-13 fr. 5-6 | 3,28-O-oleic acid diester of betulin |
| Sal-13 fr. 10-12 | 28-O-oleic acid ester of betulin |
| Sal-16 fr. 6-8 | 3,28-O-octanylic acid diester of betulin |
| Sal-16 fr. 11-13 | 28-O-octanylic acid ester of betulin |
| Sal-46 | betulin 3,28-diacetate |
| Sal-II-5 | betulin 28-acetate |
| Sal-II-9 | betulin 3-oxo-28-acetate |
| Sal-II-11 | betulinic acid |
| Sal-II-22 | 3-deoxy-2,3-didehydrobetulin |
| Sal-II-29 | 3-deoxy-2,3-didehydrobetulin 28-acetate |
| Sal-II-32 | betulonic acid |
| Sal-0 | betulin |
| Asa-XIV-160-DI | 28-N-acetylanthranilic acid ester of betulin |
| Asa-XIV-181-D | 28-nicotinic acid ester of betulin |

### Example 33

### Determination of the Antimicrobial Efficiency of Betulin Derived Compounds

The antimicrobial efficiency of betulin derived compounds against Staphylococcus aureus, Staphylococcus epidermidis, Micrococcus luteus and Bacillus subtilis was studied using a turbidometric method on a 96 well plate.

After regeneration, a suspension cultivation was prepared from the bacterial strains in the Todd-Hewitt broth. The suspension was introduced with a pipette to a 96 well plate, followed by the addition of the compound to be tested (3 parallel tests for each compound). First, stock solutions in DMSO were made of the compounds, and then, said stock solutions were diluted with the cultivation broth to give working solutions having a concentration of 1 μg/ml. Erythromycin was used as the control. Bacterial growth was monitored by measuring the absorbances of the samples at 620 nm at 0, 1, 2, 3, 4 and 24 hours. The sample plate was incubated at 37° C. in a shaker (250 rpm) between the measurements. Effects of the compounds on bacterial growth were evaluated by comparison of the growths of exposed and unexposed samples. The results are presented in Table 5 below as percent growth inhibition.

TABLE 5

| | S. | S. | S. epi- | S. epi- | B. sub- | B. sub- | M. | M. | |
|---|---|---|---|---|---|---|---|---|---|
| | aureus | aureus | dermidis | dermidis | tilis | tilis | luteus | luteus | |
| Yhdiste | 4 h | 24 h | 4 h | 24 h | 4 h | 24 h | 4 h | 24 h | |
| 1 | 2.1 | 0.0 | 0.0 | 0.0 | 0.0 | 8.9 | 8.8 | 7.6 | * |
| 5 | 51.6 | 0.0 | 52.7 | 0.0 | 102.3 | 0.0 | 91.3 | 0.0 | |
| 6 | 0.0 | 0.0 | 0.0 | 0.0 | 0.0 | 3.7 | 0.0 | 1.5 | * |
| 8 | 0.0 | 0.0 | 0.0 | 0.0 | 0.0 | 5.8 | 0.0 | 0.0 | |
| 10 | 0.0 | 0.0 | 0.0 | 1.7 | 0.0 | 2.6 | 0.0 | 2.7 | |
| 20 | 0.0 | 0.0 | 0.0 | 0.0 | 0.0 | 9.0 | 0.0 | 12.6 | |
| 21 | 0.0 | 0.0 | 0.0 | 0.0 | 0.0 | 6.9 | 0.0 | 10.1 | |
| 23 | 9.9 | 11.1 | 6.8 | 0.0 | 0.7 | 7.3 | 0.0 | 0.1 | * |
| 25 | 6.9 | 0.0 | 3.2 | 12.7 | 0.0 | 15.2 | 1.6 | 12.4 | |
| 29 | 102.6 | 103.4 | 66.6 | 20.2 | 100.8 | 54.6 | 89.9 | 60.5 | |
| 30 | 13.0 | 0.0 | 8.5 | 0.0 | 5.9 | 11.1 | 0.0 | 0.0 | |
| 31 | 53.6 | 11.7 | 100.0 | 90.8 | 95.6 | 100.0 | 96.1 | 100.0 | |
| 32 | 79.8 | 72.1 | 100.0 | 100.0 | 100.0 | 100.0 | 100.0 | 100.0 | |

* solubility problems

The compounds tested are as follows:

1=3,28-diisostearic acid ester of betulin
5=betulonic acid
6=betulin 3,28-diacetate-18,19-ene
8=28-aspartate dimethylesteramide of betulonic acid
10=3,28-dioctanic acid ester of betulin
20=3,28-C₁₈-dialkenylsuccinic acid diester of betulin
21=28-C₁₈-alkenylsuccinic acid ester of betulin
23=28-carvacrolacetic acid ester of betulin
25=betulin 3-acetate-28 mesylate
29=28-N-acetylanthranilic acid ester of betulin
30=28-cinnamic acid ester of betulin
31=erythromycin (0.1 μg/ml) (control)
32=erythromycin (1 μg/ml) (control)

### Composition Examples

Following compositions are examples of particularly preferable formulations for topical use.

### Composition Example 1

### Water-in-Oil Emulsion

| | |
|---|---|
| Active agent | 0.01-20% |
| Emulsifier¹ | 1-25% |
| Humectant² | 5-80% |
| Preserving agent³ | 0.01-0.5% |
| Water | 20-50% |

¹For instance fatty acid esters of sorbitan (e.g. sorbitan sesquioleate, sorbitan monostearate, sorbitan mono-oleate, sorbitan trioleate, sorbitan tristearate, sorbitan monolaurate, sorbitan monopalmitate), wool alcohols and monoglycerides
²For instance glycerine, propylene glycol
³For instance methyl paraben, ethyl paraben, propyl paraben, sorbic acid

### Composition Example 2

### Oil-in-Water Emulsion

| | |
|---|---|
| Active agent | 0.01-20% |
| Emulsifier¹ | 1-25% |
| Humectant² | 5-80% |
| Preserving agent³ | 0.01-0.5% |
| Water | 20-50% |

¹For instance sulfated fatty alcohols, sodium soaps, polysorbates, polyoxylic fatty acids, and esters of fatty alcohols
²For instance glycerine, propylene glycol
³For instance methyl paraben, ethyl paraben, propyl paraben, sorbic acid

### Composition Example 3

### Gel

| | |
|---|---|
| Active agent | 0.01-1% |
| Gelling agent¹ | 0.5-6% |
| Solvent² | 10-45% |
| Preserving agent³ | |
| Water | 20-50% |

¹For instance starch, cellulose derivatives, carbomers, and magnesium aluminium silicates
²For instance ethanol, isopropanol
³For instance methyl paraben, ethyl paraben, propyl paraben, sorbic acid

### Composition Example 4

### Ointment

| | |
|---|---|
| Active agent | 0.01-20% |
| Ointment base¹ | 1-25% |
| Preserving agent² | 0.01-0.5% |

¹For instance liquid paraffins, plant oils, animal fats, synthetic glycerides, macrogols
²For instance methyl paraben, ethyl paraben, propyl paraben, sorbic acid

### Composition Example 5

### Oil-in-Water Emulsion

| | |
|---|---|
| Active agent | 1.0% |
| Cetostearyl alcohol | 25.0% |
| Glycerine | 4.0% |
| Glyceryl monostearate | 4.8% |
| Methyl paraben | 0.1% |
| Propyl paraben | 0.1% |
| Water | 65.0% |

### Composition Example 6

### Water-in-Oil Emulsion

| | |
|---|---|
| Active agent | 1.0% |
| Stearyl alcohol | 35.0% |
| Macrogol stearate | 8.0% |
| Propylene glycol | 10.0% |
| Mineral oil | 5.0% |
| Methyl paraben | 0.1% |
| Propyl paraben | 0.1% |
| Water | 40.8% |

### Composition Example 7

### Ointment

| | |
|---|---|
| Active agent | 1.0% |
| Vaseline | 63.8% |
| Liquid paraffin | 15.0% |
| Glyceryl stearate | 10.0% |
| Propylene glycol | 10.0% |
| Sorbic acid | 0.2% |

### Composition Example 8

### Gel

| | |
|---|---|
| Active agent | 1.0% |
| Carbomer | 3.0% |
| Glycerine | 10.0% |
| Ethanol | 33.9% |
| Water | 53.0% |

### Composition Example 9

### Multi-Vitamin Cream

%, by weight

A

| | |
|---|---|
| PEG-7 hydrogenated castor oil | 6.00 |
| Paraffin oil/mineral oil | 10.00 |
| Vaseline | 3.00 |
| Caprylic/capric triglyceride | 5.00 |
| PEG-45/dodecylglycol copolymer | 2.00 |
| Jojoba oil/Jojoba (Buxus chinensis) oil | 5.00 |
| Quaternium-18 bentonite | 1.00 |

B

| | |
|---|---|
| 10% betulonic acid in propylene glycol | 3.00 |
| EDTA | 0.10 |
| Preserving agent | q.s. |
| Water | 62.90 |

C

| | |
|---|---|
| Sodium ascorbyl phosphate | 1.00 |
| Retinol | 1.00 |
| Perfume | q.s. |

The polyvitamin cream is prepared by separately heating the ingredients of phases A and B to about 80° C. Phase B is stirred into phase A while homogenizing, homogenization being continued for a while. The mixture is cooled to about 40° C., ingredients of phase C are added, and homogenization is repeated. The viscosity of the composition is about 14 000 mPas (Haake Viscotester VT-02).

### Composition Example 10

### Sun Screen Foam

%, by weight

A

| | |
|---|---|
| Cremophor A 25/Ceteareth-25 | 5.00 |
| Palmitic acid | 2.00 |
| Alkyl benzoate | 5.00 |
| PPG-3 myristyl ether | 5.00 |
| Octylmethoxy cinnamate | 6.00 |
| Octyl triazone | 0.50 |
| 4-methylbenzylidene camphore | 1.00 |

B

| | |
|---|---|
| 10% betulonic acid in pentylene glycol | 5.00 |
| Preserving agent | q.s. |
| Water | 70.30 |

C

| | |
|---|---|
| Triethanol amine | 0.20 |
| Perfume | q.s. |

The ingredients of phases A and B are separately heated to about 80° C. Phase B is stirred into phase A while homogenizing. Ingredients of phase C are added, and homogenization is repeated. The mixture is cooled to about 40° C., ingredients of phase D are added, and homogenization is repeated. Filling: 90% of the active ingredient, 10% of propane/butane mixture at the pressure of 3.5 bar (20° C.).

### Composition Example 11

### Soft Cream with Vitamin E

%, by weight

A

| | |
|---|---|
| Polyglyceryl 3-dioleate | 0.75 |
| Cetearyl octanoate | 7.50 |
| Alkyl benzoate | 5.00 |
| Caprylic/capric triglyceride | 4.00 |
| Cetyl diethicone copolyol | 2.25 |
| Dimethicone | 1.50 |
| BHT, ascorbyl palmitate, citric acid, | 0.20 |
| glyceryl stearate, propylene glycol | |

B

| | |
|---|---|
| 5% betulonic acid in TEA lactate | 0.75 |
| Sodium hydroxide | 0.25 |
| Panthenol | 1.50 |
| Sodium chloride | 1.50 |
| EDTA | 0.1 |
| Preserving agent | q.s. |
| Water | 69.80 |

C

| | |
|---|---|
| (−)-Alpha-bisabolole nat./bisabolol | 0.10 |
| Vitamin A palmitate 1 Mio./retinyl palmitate | 0.10 |
| Vitamin E acetate/tocopheryl acetate | 5.00 |
| Perfume | q.s. |

The ingredients of phases A and B are separately heated to about 80° C. Phase B is stirred into phase A while homogenizing. The mixture is cooled to about 40° C., ingredients of phase C are added, and homogenization is repeated. Viscosity of the composition is about 18 000 mPas.

### Composition Example 12

### Sun Protection Gel

%, by weight

A

| | |
|---|---|
| Octylmethoxy cinnamate | 8.00 |
| Octocrylene | 5.00 |
| Benzophenone-3 | 2.00 |
| Butylmethoxy dibenzoylmethane | 0.80 |
| Vitamin E acetate/tocopheryl acetate | 2.00 |
| PEG-40 hydrogenated castor oil | 1.00 |
| Perfume | q.s. |

B

| | |
|---|---|
| Acrylates/C₁₀₋₃₀ alkylacrylate crosspolymer | 0.30 |
| Carbomer | 0.20 |
| 10% betulonic acid in dioctylglycol | 5.00 |
| EDTA | 0.20 |
| Preserving agent | q.s. |
| Water | 75.30 |

C

| | |
|---|---|
| Sodium hydroxide | 0.20 |

Ingredients of phase A are dissolved. Ingredients of phase B are stirred into phase A while homogenizing, followed by neutralization with ingredients of phase C, and homogenization is repeated. Viscosity of the composition is about 5 500 mPas (Haake Viscotester VT-02), pH value being about 9.1.

### Composition Example 13

### Compact Powder

%, by weight

A

| | |
|---|---|
| Talc | 72.00 |
| Magnesium stearate | 10.00 |
| Calcium carbonate | 2.00 |
| Titanium dioxide | 9.00 |
| Iron oxides | 1.00 |
| Powder containing betulonic acid | 5.00 |

B

| | |
|---|---|
| Paraffin oil/mineral oil | 0.50 |
| Vaseline | 0.50 |

The powder is prepared by mixing and homogenizing the ingredients of phase A. Ingredients of phase B are added. The mixture is compressed at 40° C.

### Composition Example 14

### Fluid Foundation with Granlux® Melanin Mimic™ TB Concentrate

| A | Amount (%) |
|---|---|
| Magnesiumaluminium silicate | 0.70 |
| Xanthane gum | 0.30 |
| 10% betulonic acid in propylene glycol | 6.00 |
| Glycerine | 4.00 |
| Deionized water | q.s. |

Xanthane gum is wetted in the mixture of water+glycerine+10% betulonic acid in propylene glycol. The mixture is homogenized with a turboemulsifier, and then magnesiumaluminium silicate is added while mixing, and heated to 75° C.

B

| | |
|---|---|
| Granlux ® Melanin Mimic ™ TB | 27.50 |
| Limnanthes Alba; | 3.50 |
| Butyrospermum Parkii | |
| Glyceryl stearate | 0.80 |
| Isopropyl myristate | 4.00 |
| Isohexadecane | 10.00 |
| Stearic acid | 2.00 |
| Dimethicone (Dow Corning) | 1.00 |

Ingredients of phase B are melted at 65° C., slowly homogenized for about 5 minutes and heated to 75° C.

B1

| | |
|---|---|
| Talc | 1.00 |

Ingredients of phase A are added to phase B while homogenizing. Once an emulsion has been formed, ingredients of phases B1 and C are slowly added with constant homogenization.

C

| | |
|---|---|
| Triethanol amine | 1.50 |

D

| | |
|---|---|
| PPG 25 Laureth 25 (Vevy) | 0.20 |
| Propylene glycol; diazolidinyl urea; | 1.00 |
| methylparaben; propylparaben (ISP) | |

Ingredients of phase D are added at 40° C. while homogenizing. The mixture is cooled to room temperature while mixing.

### Characteristics:

pH about 7

### Viscosity: 6000

### SPF: 21-24

### Composition Example 15

### Soft Coloured Cream (SCC/EM/98)

| A | Amount (%) |
|---|---|
| Magnesiumaluminium silicate | 0.50 |
| Xanthane gum | 0.50 |
| Propylene glycol | 6.00 |
| 10% betulonic acid in glycerine | 4.00 |
| Deionized water | up to 100% |

Xanthane gum is wetted in the mixture of water+glycerine+propylene glycol. The mixture is homogenized with a turboemulsifier, and then magnesiumaluminium silicate is added while mixing, and heated to 75° C.

B

| | |
|---|---|
| Granlux ™ EM-50 (Granula Ltd) | 10.00 |
| Butyrospermum Parkii | 3.50 |
| Glyceryl stearate | 0.80 |
| Isopropyl myristate | 4.00 |
| Isohexadecane | 10.00 |
| Polydecene | 4.00 |
| Polyhydroxystearic acid | 0.50 |

The ingredients of phase B are melted at 65° C., the ingredients of phase B1 are added while slowly homogenizing for about 5 minutes, followed by heating to 75° C.

B1

| | |
|---|---|
| Ariabel yellow, Warner & Jenkinson | 1.40 |
| Ariabel sienna, Warner & Jenkinson | 0.30 |
| Ariabel umber, Warner & Jenkinson | 0.30 |
| Titanium dioxide | 6.00 |

Ingredients of phase A are added to the ingredients of phases B and B1 while homogenizing. Once an emulsion is formed, ingredients of phase C are added using constant homogenization.

C

| | |
|---|---|
| Talc | 1.00 |
| Aluminium starch octenyl succinate | 3.00 |

D

| | |
|---|---|
| PPG 25 Laureth 25 (Vevy) | 0.20 |
| Propylene glycol; diazolidinyl urea; | 1.00 |
| methylparaben; propylparaben (ISP) | |

Ingredients of phase D are added at 40° C. while homogenizing. The mixture is cooled to room temperature while mixing. Note: during formulation, the phase inversion temperature (PIT) may be clearly seen. (PIT is about 40° C.) since the water-in-oil emulsion formed earlier separates to give two phases: liquid and creamy. The final oil-in-water emulsion is readily obtained by continuing homogenization. Low PIT value is not associated with instability, in fact, the formulation is still stable after storage for 4 months at 42° C.

### Characteristics:

pH about 7
Viscosity: 180 000 mPas RVT Brookfield (5 rpm, 298 K, Helipath Stand T-D)
SPF: 21-23 in vitro, UVA/UVB=0.77

### Composition Example 16

### Cell Protective Composition

| | |
|---|---|
| Ingredients | [%] |

A

| | |
|---|---|
| Ectoin | 1.00 |
| 10% betulonic acid in glycerol | 3.00 |
| Preserving agents | q.s. |
| Water | to 100 |

B

| | |
|---|---|
| Sucrose distearate | 2.70 |
| Sucrose stearate | 0.90 |
| Dicaprylic ether | 5.00 |
| Caprylic/capric glyceride | 2.00 |
| Isopropyl palmitate | 2.00 |
| Ethylhexyl palmitate | 7.00 |
| Carbomer | 0.20 |

C

| | |
|---|---|
| Sodium hydroxide | q.s. |

Ingredients of phase A are heated to 75° C., ingredients of phase B are dispersed and heated to 75° C., ingredients of phase B are added to the ingredients of phase A, homogenized, pH-value is adjusted with sodium hydroxide, cooled to room temperature while stirring. pH (22° C.): 6.50, viscosity (21° C.): 109 000 mPas (Brookfield RVT, spindle C, 5 rpm, Helipath).

### Composition Example 17

### Body Milk

%, by weight

A

| | |
|---|---|
| Ceteareth-6, stearyl alcohol | 1.00 |
| Ceteareth-25 | 1.00 |
| Glyceryl monostearate | 2.00 |
| Cetyl stearyl alcohol | 2.00 |
| Paraffin oil/mineral oil | 3.00 |
| Cetearyl octanoate | 5.00 |

B

| | |
|---|---|
| 10% betulonic acid in propylene glycol | 5.00 |
| Polyquaternium-11 | 4.00 |
| Preserving agent | q.s. |
| Water | 77.00 |

C

| | |
|---|---|
| Perfume | q.s. |

Ingredients of phases A and B are separately heated to about 80° C. Ingredients of phase B are stirred into ingredients of phase A while homogenizing, homogenization being continued for a while. The mixture is cooled to about 40° C., ingredients of phase C are added, and homogenization is repeated. Viscosity: about 3000 mPas, pH value: about 6.

### Composition Example 18

### Aftersun Rehydrating Body Spray

| | |
|---|---|
| Ingredient | %, by weight |

A

| | |
|---|---|
| Deionized water | 89.10 |
| Hydroxy ethyl cetyldimonium phosphate | 2.00 |
| D-panthenol (BASF) | 0.50 |
| 10% betulonic acid in propylene glycol | 5.00 |
| Dimethicone copolyol | 0.50 |
| Sodium lactate & sodium PCA & sorbitol & | 2.00 |
| hydrolyzed collagen & proline | |
| Nipaguard ® DMDMH (DMDM hydantoin) (Nipa) | 0.50 |

B

| | |
|---|---|
| PEG-40 hydrogenated castor oil | 0.30 |
| Fragrance | 0.10 |

Ingredients of phase A are mixed together and stirred to give a clear mixture. Ingredients of phase B are mixed together. Hydrogenated castor oil is melted and mixed with fragrance. Ingredients of phase B are added to ingredients of phase A and mixed to give a clear mixture. pH of the final product is 6.

### Composition Example 19

### After Shave Gel without Alcohol

%, by weight

A

| | |
|---|---|
| Carbomer | 0.30 |
| Demineralized water | 40.00 |

B

| | |
|---|---|
| PEG-40/hydrogenated castor oil | 3.00 |
| Perfume | q.s. |
| Menthol | 0.10 |
| D-panthenol 50 P/panthenol | 0.10 |
| 10% betulonic acid in propylene glycol | 4.00 |
| Triethanol amine | 0.40 |
| Preserving agent | q.s. |
| Demineralized water | 52.20 |

Ingredients of phase A are allowed to swell. Ingredients of phase B are dissolved and stirred with ingredients of phase A. Viscosity: about 4 000 mPas (Brookfield RVT), pH value about 7.

### Composition Example 20

### Cream with High Protection Factor

Amount (%)

A

| | |
|---|---|
| GranLux ® GAI-45 TS (Granula Ltd) | 25.0 |
| 10% betulonic acid in pentylene glycol dispersion | 3.0 |

B

| | |
|---|---|
| Water | 10.0 |
| Nipagin M, Germail | 0.1 |

C

| | |
|---|---|
| Isononyl isononanoate | 22.0 |

D

| | |
|---|---|
| Water | 39.0 |
| Perfume | q.s. |

1) A is mixed at room temperature.
2) B is prepared and added to A. The mixture is mixed for about 3 to 5 min until all water has been taken up. Water is retained by diffusion, thus hydrating polar parts and forming a liquid crystalline phase. Initially, the polar phase and the hydrophobic phase seem to be totally separated but by time and mixing the water phase will be taken up.
3) C is added to the mixture of A+B, while mixing. Viscosity is lowered.
4) D is slowly added to the mixture of C+A+B (during about 5 minutes), proceeding carefully for a total processing time of 15 min. (Ystral speed 3 to 5).
SPF: well over 30 (SPF in vitro 49-3)
UVA: fulfills Australian standard.

### Composition Example 21

### Moisturizing Cream with High Protection Factor

| A | Amount (%) |
|---|---|
| Granlux ® EM-50 (Granula Ltd) | 10 |
| Heptanoic triglyceride | 20 |
| Dimethicone | 5 |
| 4-methylbenzylidene camphor | 5 |
| Butylmethoxy dibenzoylmethane | 2 |

Melt A and heat the mixture to 70° C.

B

| | |
|---|---|
| Magnesiumaluminium stearate | 1 |
| 10% dispersion of betulonic acid in butylene glycol | 2.0 |
| Water | 58.2 |

B is separately warmed and A is added while continuously emulsifying with a suitable mixer.

C

| | |
|---|---|
| Cyclomethicone (Dow Corning) | 5 |

C (volatile silicone) is added at 60° C.

D

| | |
|---|---|
| Phenoxyethanol/C₁/C₂/C₃/C₄-alkyl paraben | 0.50 |
| Perfume | 0.30 |

D (preserving agent and perfume) is added at 40° C.

### Characteristics:

Appearance: smooth shiny cream
pH: about 7
SPF: 30 to 35 in vitro

### Composition Example 22

### Cream with High Protection Factor (SPF=20)

Amount (%)

A

| | |
|---|---|
| GranLux ® GAI-45 (Granula Ltd) | 9.45 |
| Polyglycerol-4-isostearate (and) cetyl | 4.00 |
| Dimethicone copolyol (and) hexyl laureate | |
| Isononyl isononanoate | 18.95 |
| Cyclomethicone | 7.50 |
| Cetyl dimethicone | 3.00 |
| Methylglucosides sesquistearate | 0.50 |
| Tridecyl neopentanoate | 2.00 |
| Nonsaponifiable constituents of | 4.00 |
| hydrogenated olive oil (and) olive oil | |
| unsaponifiables | |
| Sorbitan olivate | 3.00 |

B

| | |
|---|---|
| Water | 40.80 |
| Xanthane gum | 0.20 |
| 10% betulonic acid in butylene glycol | 3.00 |
| Sodium chloride | 0.50 |
| PEG-150 copolymer | 2.50 |

C

| | |
|---|---|
| Phenoxyethanol and methyl paraben and | 0.60 |
| ethyl paraben and propyl paraben and | |
| butyl paraben | |

1) Mix B with a propeller at room temperature.
2) Once B is completely dissolved, add the premixture C to B.
3) First heat A to 80° C., then cool to 65° C., and homogenize.
4) Using the propeller, add step 2) to step 4).
pH: 7.05
SPF in vitro=20

### Composition Example 23

### Oil-in-Water Lotion Comprising Granlux® TEM-45

%, by weight

Phase A

| | |
|---|---|
| 10% betulonic acid in glycerine | 3.0 |
| Xanthane gum | 0.3 |
| EDTA | 0.2 |
| Water | q.s. |

Phase B

| | |
|---|---|
| Granlux ® TEM-45 (Granula) | 12.0 |
| Octylmethylcinnamate | 4.0 |
| Butylmethoxy dibenzoylmethane | 1.5 |
| C12-C15-alkylbenzoate | 5.0 |
| Methylglycose sesquistearate | 3.0 |
| Dimethicone | 0.5 |

Phase C

Perfume, preserving agents as desired.

Dissolve xanthane gum as described by the manufacturer to ingredients of phase A. Heat the ingredients of the phases A and B to 75° C. while agitating. Then mix the ingredients of the phases A and B, and homogenize. Once the temperature is below 30° C., add the selected preserving agents and perfumes as desired. Expected SPF+20.

### Composition Example 24

### SPF 15 Stick

| | Amount (%) |
|---|---|
| Hydrogenated vegetable oil | 15.0 |
| Vegetable oil | 68.0 |
| Candelilla wax | 6.0 |
| Betulonic acid | 1.0 |
| Granlux CCA-50 (Oy Granula) | 10.00 |

Heat the ingredients to 75 to 80° C. Mix to give a uniform mixture. Cool to 50° C. Pour into moulds.

### Characteristics:

SPF: 13-15 in vitro
UVA/UVB ratio: 0.56

### Composition Example 25

### SPF 30 Stick Composition

| | Amount (%) |
|---|---|
| Beeswax | 12.0 |
| Caprylic/capric triglycerides | 12.5 |
| Macadamia nut oil | 9.5 |
| Cetearyl alcohol | 7.5 |
| Petrolatum | 36.5 |
| Granlux CCA-50 (Granula) | 20.00 |
| Betulonic acid | 2.0 |

Heat the ingredients to 75 to 80° C. Mix to give a uniform mixture. Pour into moulds.

### Characteristics:

SPF: 28-30 in vitro

### Composition Example 26

### Night Cream

%, by weight

A

| | |
|---|---|
| PEG-7 hydrogenated castor oil | 6.00 |
| Cetearyl octanoate | 5.00 |
| Microcrystalline wax | 2.00 |
| Beeswax | 0.50 |
| Shea butter (Butyrospermum Parkii) | 0.50 |
| Jojoba oil/Jojoba (Buxus Chinensis) oil | 2.00 |
| Paraffin oil/mineral oil | 10.00 |

B

| | |
|---|---|
| 10% betulonic acid in propylen glycol | 5.00 |
| Preserving agent | q.s. |
| Water | 67.00 |

C

| | |
|---|---|
| Sodium ascorbyl phosphate | 2.00 |
| Perfume | q.s. |

Heat the ingredients of phases A and B separately to about 80° C. Add phase B to phase A while homogenizing, homogenization being then continued for a while. Cool to about 40° C., add the ingredients of phase C, and homogenize again. Viscosity about.

### Composition Example 27

### Oil-in-Water Type UVA/UVB Sun Protection Lotion with TINOSORB® M

Lotion having a very high SPF and providing an excellent UVA protection due to photostable UVA filter TINOSORB® M. This emulsion is smooth and spreads easily. SPF in vivo=38, broadband.

| | |
|---|---|
| Composition | %, by weight |

Part A

| | |
|---|---|
| Potassium cetylphosphate | 2.00 |
| Tricontanyl PVP | 1.00 |
| Caprylic/capric triglyceride | 5.00 |
| C₁₂₋₁₅ alkylbenzoate | 5.00 |
| Cetearyl isononanoate | 5.00 |
| Glyceryl stearate | 3.00 |
| Cetyl alcohol | 1.00 |
| Dimethicone | 0.10 |
| Ethylhexyl methoxy cinnamate | 5.00 |

Part B

| | |
|---|---|
| Water | q.s. to 100 |
| 10% betulonic acid in glycerine | 3.00 |

Part C

| | |
|---|---|
| Steareth-10 allylether/acrylates copolymer | 0.50 |

Part D

| | |
|---|---|
| Methylene bis-benzotriazolyl tetramethyl butyl phenol | 20.00 |
| (and) water (and) decyl glucoside (and) | |
| propylene glycol (and) xanthane gum | |

Part E

| | |
|---|---|
| Phenoxyethanol (and) methyl paraben (and) | 1.00 |
| ethyl paraben (and) butyl paraben (and) | |
| propyl paraben (and) isobutyl paraben | |

Part F

| | |
|---|---|
| Sodium hydroxide (10% solution) | q.s. to a pH |
| | value of 7.00 |

Part G

| | |
|---|---|
| Perfume | q.s. |
| Technical data: | |
| pH value | 7.00 |
| Appearance: | while lotion |
| Viscosity (Brookfield DVIII + LV4/80 rpm) | 3000 mPas |
| UVA/UVB ratio*/critical wave length* | 0.75/384 nm |

1-62. (canceled)

63. A lipstick or a sun protection composition, characterized in that it comprises between 0.01 and 20% by weight of betulonic acid and optionally one or more betulin derivative(s) formulated with one or more constituent(s) or excipient(s) selected from the group consisting of additives, fillers, carriers, vectors, surfactants, solvents, UV protection agents, antioxidants, preserving agents, colouring agents, alcohols, waxes, oils, fats, perfumes, thickeners and pharmaceutically and/or cosmetically active agents.

64. Composition according to claim 63, characterized in that it comprises between 0.1 and 10% by weight of betulonic acid.

65. Composition according to claim 63, characterized in that it comprises between 0.01 and 20% by weight of one or more betulin derivatives.

66. Composition according to claim 63, characterized in that the betulin derivatives are selected from the group consisting of compounds of the general formula I and pharmaceutically acceptable salts thereof, where 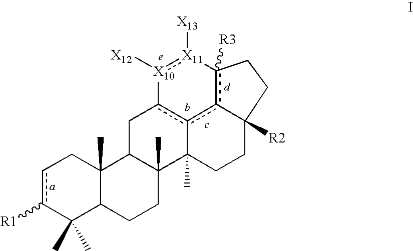
A. R1=OH;
B. R2=CH₂O(C═O)CH₂(CHR_{g})COOY where R_{g}═C₄-C₂₂ linear or branched alkyl or alkenyl group, Y═H, Na, K, Ca, Mg, C₁-C₄-alkyl group or NRₕ where Rₕ═H or C₁-C₄-alkyl group;
C. R3=CH₂═CCH₃; and
D. X₁₀═X₁₁═H, X₁₂═X₁₃=absent, a, b, c, and d each represent a single bond and e=absent; or
R1=OH;
R2=CH₂ORᵢ where Rᵢ=2,5-diaminopentanoyl, nicotinoyl, 2-(acetylamino)benzoyl, N,N,N-trimethyl-2-oxoethanaminium or isostearoyl group;
R3=CH₂═CCH₃; and
X₁₀═X₁₁═H, X₁₂═X₁₃=absent, a, b, c, and d each represent a single bond and e=absent; or
R1=OH;
R2=CH₂ORₙ, or CH₂O(C═O)CH₂OR′ where R′=verbenzyl, terpinyl, thymyl, carvacryl, menthyl, cinnamyl, curcuminyl, eugenyl, bornyl, isobornyl, longifolyl, isolongifolyl, globutyl, epiglobutyl, cedryl, or epicedryl group and Rₙ=chrysanthemoyl, cinnamoyl or retinoyl group; and
R3=CH₂═CCH₃; and
X₁₀═X₁₁═H, X₁₂═X₁₃=absent, a, b, c, and d each represent a single bond and e=absent; or
R1=O(C═O)CH₂(CHR_{c})COOY where R_{c}═C₄-C₂₂ linear or branched alkyl or alkenyl group, Y═H, Na, K, Ca, Mg, C₁-C₄ alkyl group or NRₕ where Rₕ═H or a C₁-C₄ alkyl group;
R2=CH₂O(C═O)CH₂(CHR_{d})COOY where R_{d}═C₄-C₂₂ linear or branched alkyl or alkenyl group, Y═H, Na, K, Ca, Mg, C₁-C₄ alkyl group or NRₖ where Rₖ═H or a C₁-C₄ alkyl group;
R3=CH₂═CCH₃; and
X₁₀═X₁₁═H, X₁₂═X₁₃=absent, a, b, c, and d each represent a single bond and e=absent; or
R1=ORᵣ where Rᵣ=2,5-diaminopentanoyl, nicotinoyl, 2-(acetylamino)benzoyl, N,N,N-trimethyl-2-oxoethanaminium or isostearoyl group;
R2=CH₂ORₚ where Rₚ=2,5-diaminopentanoyl, nicotinoyl, 2-(acetylamino)benzoyl or N,N,N-trimethyl-2-oxoethanaminium or isostearoyl group;
R3=CH₂═CCH₃; and
X₁₀═X₁₁═H, X₁₂═X₁₃=absent, a, b, c, and d each represent a single bond and e=absent; or
R1=ORᵥ or O(C═O)CH₂OR′ where R′=verbenzyl, terpinyl, thymyl, carvacryl, menthyl, cinnamyl, curcuminyl, eugenyl, bornyl, isobornyl, longifolyl, isolongifolyl, globutyl, epiglobutyl, cedryl or epicedryl group and Rᵥ=chrysanthemoyl, cinnamoyl or retinoyl group;
R2=CH₂ORᵤ or CH₂O(C═O)CH₂OR′ where R′=verbenzyl, terpinyl, thymyl, carvacryl, menthyl, cinnamyl, curcuminyl, eugenyl, bornyl, isobornyl, longifolyl, isolongifolyl, globutyl, epiglobutyl, cedryl or epicedryl group and Rᵤ=chrysanthemoyl, cinnamoyl or retinoyl group;
R3=CH₂═CCH₃; and
X₁₀═X₁₁═H, X₁₂═X₁₃=absent, a, b, c, and d each represent a single bond and e=absent; or
R1=OH,
R2=(C═O)NHCHRₓ,COOY where Y═H, Na, K, Ca, Mg, C₁-C₄ alkyl group or NR_{y} where R_{y}═H or a C₁-C₄ alkyl group, and Rₓ═H, C₁-C₄-alkyl, benzyl, 4-hydrozybenzyl, 4-imidazolylmethyl or 3-indolylmethyl group, or L-aspartate, L-histidine, L-glutamine or L-lysine residue;
R3=CH₂═CCH₃; and
X₁₀═X₁₁═H, X₁₂═X₁₃=absent, a, b, c, and d each represent a single bond and e=absent; or
R1=oxo group;
R2=(C═O)NHCHRₓCOOY where Y═H, Na, K, Ca, Mg, C₁-C₄ alkyl group or NR_{y} where R_{y}═H or a C₁-C₄ alkyl group, and Rₓ═H, C₁-C₄ alkyl, benzyl, 4-hydroxybenzyl group, CH₂CH₂CH₂CH₂NH₂, 4-imidazolylmethyl, 3-indolylmethyl, CH₂COOZ or CH₂CH₂COOZ group where Z═R_{y};
R3=CH₂═CCH₃; and
X₁₀═X₁₁═H, X₁₂═X₁₃═absent, a, b, c, and d each represent a single bond and e=absent; or
R1=oxo group;
R2=(C═O)OR_{w}, where R_{w}=verbenzyl, terpinyl, thymyl, carvacryl, menthyl, cinnamyl, curcuminyl, eugenyl, bornyl, isobornyl, longifolyl, isolongifolyl, globutyl, epiglobutyl, cedryl or epicedryl group
R3=CH₂—CCH₃ or CH₃—CH—CH₃; and
X₁₀═X₁₁═H, X₁₂═X₁₃=absent, a, b, c, and d each represent a single bond and e=absent; or
R1=OH or O—(C═O)R_{b} where R_{b}═C₃-C_{g} cyclic or heterocyclic residue, substituted or unsubstituted phenyl or benzyl residue or C₁-C₂₂ alkyl or alkenyl group;
R2=CH₂OH or CH₂O—(C═O)R_{f} where R_{f}═C₃-C_{g} cyclic or heterocyclic residue, substituted or un substituted phenyl or benzyl residue or C₁-C₂₂ alkyl or alkenyl;
R3=H₂C═CCH₂R_{q} or CH₃CCH₂R_{q} where R_{q}=3-dihydrofuran-2,5-dione or 3-pyrrolidine-2,5-dione or CH(COORo)CH₂COORz where Rₒ═H, Na, K, Ca, Mg or a C₂₂ linear or branched alkyl or alkenyl group and R_{z}═H, Na, K, Ca, Mg or a C₁-C₂₂ linear or branched alkyl or alkenyl group; and
X₁₀═X₁₁═H, X₁₂═X₁₃=absent, a, b, c, and d each represent a single bond and e=absent; or
R1=H, OR_{z}, O(C═O)R_{b}, NRₐR_{z}, CN, ═NORₐ, CHO, (C═O)OR_{z}, SR_{z}, ═O, ═S, where R_{z}═H, C₁-C₆ linear or branched alkyl or alkenyl group or an aromatic group ZZ shown below, and Rₐ═H, C₁-C₆ linear or branched alkyl or alkenyl group or an aromatic group ZZ, and R_{b}═H, C₁-C₂₂ linear or branched alkyl or alkenyl group or an aromatic group ZZ, or R₁ corresponds to the partial structure XX shown below;
R2 ═CH₂OR_{z}, CH₂O(C═O)R_{b}, (C═O)OR_{b}, CH₂NRₐR_{z} CH₂CN, CH═NORa, CN, CH₂CHO, CH₂(C═O)OR_{z}, CH₂SR_{z}, CH—O, CH═S where R_{z}═H, C₁-C₆ linear or branched alkyl or alkenyl group or an aromatic group ZZ, and Rₐ═H, C₁-C₆ linear or branched alkyl or alkenyl group or an aromatic group ZZ, and R_{b}═C₁-C₂₂ linear or branched alkyl or alkenyl group or an aromatic group ZZ, or R2 corresponds to the partial structure YY shown below;
R3=CH₂═C—CH₃ or CH₃—CH—CH₃;
X₁₀═X₁₁═H; X₁₂═X₁₃=“absent”; a, b, c, and d independently represent a single or a double bond and e=“absent”;
said partial structures XX and YY, where YY═CH₂XX being selected from the group consisting of: 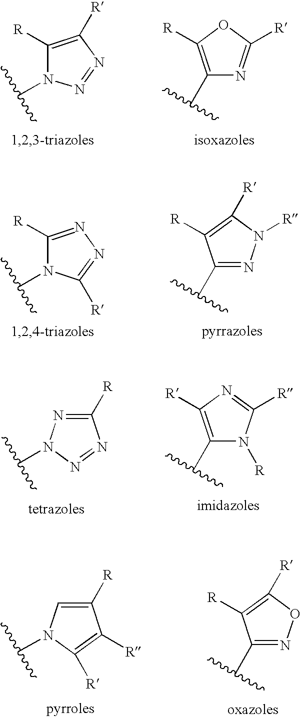
in which structures R, R′, and R″ independently represent H, an aromatic group ZZ, C₁-C₆ linear or branched alkyl or alkenyl group; the aromatic group ZZ being of the form: 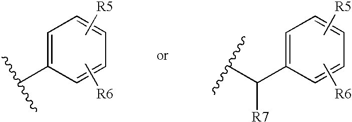
where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen, nitro, carboxy, carboxyl, acetyl, R5-R6 forms a cyclic methylenedioxide group, sulfate, cyano, hydroxy or trifluoromethyl group; or
R1=H, OR_{z}, NRₐR_{z}, CN, CHO, (C═O)OR_{z}, O(C═O)R_{b}, O(C═O)NHR_{f}, SR_{z}, ═O, or ═S where R_{z}═H, C₁-C₆ linear or branched alkyl or alkenyl group or an aromatic group ZZ, and Rₐ═H, C₁-C₆ linear or branched alkyl or alkenyl group or an aromatic group ZZ, and R_{b}═H, C₁-C₂₂ linear or branched alkyl or alkenyl group or an aromatic group ZZ, or R_{b} corresponds to the partial structure YX shown below, and R_{f}═H, C₁-C₆ linear or branched alkyl or alkenyl group or an aromatic group ZZ or R_{f} corresponds to the partial structure YX shown below;
R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{f}, CH₂SR_{z}, CH═O, CH═S where R_{z}═H, C₁-C₆ linear or branched alkyl or alkenyl group or an aromatic group ZZ, and Rₐ═H, C₁-C₆ linear or branched alkyl or alkenyl group or an aromatic group ZZ, and R_{b}═C₁-C₂₂ linear or branched alkyl or alkenyl group or an aromatic group ZZ, or R_{b} corresponds to the partial structure YX shown below, and R_{f}═H, C₁-C₆ linear or branched alkyl or alkenyl group or an aromatic group ZZ, or R_{f} corresponds to the partial structure YX shown below;
R3=CH₂═C—CH₃ or CH₃—CH—CH₃; X₁₀═X₁₁═H, X₁₂═X₁₃=“absent”; a, b, c, and d independently represent a double or a single bond; e=absent; and said aromatic group ZZ is of the form: 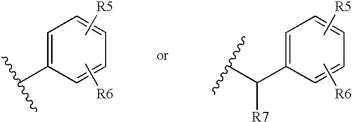
where R5, R6 and/or R7 is H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen, nitro, carboxy, carboxyl, acetyl, R5-R6 forms a cyclic methylenedioxy group, sulfate, cyano, hydroxy or trifluoromethyl; and the partial structure R_{f} or R_{b} is of the form YX: 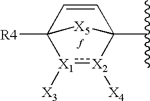
where R4=H or a C₁-C₂₀ linear or branched alkyl or alkenyl group or an aromatic group ZZ; X₅=“absent”, C, O, N or S; X₁—X₂ forms a cyclic partial structure of the form: X₁—(X₃═X₆)—X₇—(X₄═X₈)—X₂ where X₁═X₂═C or N; X₃═X₄═C; X₆═X₈═O, S or “absent”; X₇═C, O, S, or N—X₉ where X₉═H, C₁-C₆ linear or branched alkyl or alkenyl group or an aromatic group ZZ; and f is a single or a double bond; or
R1=H, OR_{z}, NRₐR_{z}, CN, CHO, (C═O)OR_{z}, O(C═O)R_{b}, O(C═O)NHR_{z}, SR_{z}, ═O, or ═S where R_{z}═H, C₁-C₆ linear or branched alkyl or alkenyl group or an aromatic group ZZ, and Rₐ═H, C₁-C₆ linear or branched alkyl or alkenyl group or an aromatic group ZZ, and R_{b}═H, C₁-C₂₂ linear or branched alkyl or alkenyl group or an aromatic group ZZ;
R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{z}, CH₂SR_{z}, CH═O, CHS where R_{z}═H, C₁-C₆ linear or branched alkyl or alkenyl group or an aromatic group ZZ and Rₐ═H, C₁-C₆ linear or branched alkyl or alkenyl group or an aromatic group ZZ, and R_{b}═C₁-C₂₂ linear or branched alkyl or alkenyl group or an aromatic group ZZ;
R3=CH₂═C—CH₃ or CH₃—CH—CH₃;
ZZ is of the form: 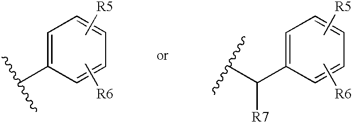
where R5, R6 and/or R7 may be H, a C₁-C₆ linear or branched alkyl or alkenyl group, a C₆ linear or branched alkyl or alkenyl ether, R5-R6 forms a cyclic C₂-C₅ alkyl or alkenyl group, halogen, nitro, carboxyl, acetyl, R5-R6 forms a cyclic methylenedioxy group, sulfate, cyano, hydroxy or trifluoromethyl; at X₁₀—X₁₁, a cyclic or heterocyclic partial structure having the form X₁₀—(X₁₂═X₁₄)—X₁₅—(X₁₃═X₁₆)—X₁₁ is present where X₁₀═X₁₁═C or N; X₁₂═X₁₃═C; X₁₄═X₁₆═O, S or absent; X₁₅═C, O, S, or N—X₁₇ where X₁₇═H, a C₁-C₆ linear or branched alkyl or alkenyl group or an aromatic group ZZ; and a, b, c, d and e independently represent double or single bonds; and
betulin, betulin 28-acetic acid methyl ester, 20,29-dihydrobetulin, 20,29-dihydrobetulonic acid, betulinic aldehyde, betulonic aldehyde, betulinic acid, 3-deoxy-2,3-dihydrobetulin, betulin 28-oxime, betulin 3,28-dioxime and betulin 3-acetoxyoxime-28-nitrile.

67. The composition according to claim 63, characterized in that the betulin derivative is selected from the group consisting of betulin 3,28-C₁₈-dialkenylsuccinic acid diester, betulin-28-yl (5-isopropyl-2-methyl-phenoxy)acetate, betulinic acid, betulin-3,28-yl bis(N,N,N-trimethyl-2-oxoethanaminiumyl), betulin 3-acetate-28-mesylate, betulin-28-yl 2-(acetylamino)benzoate, and betulin-28-yl cinnamate.

68. The composition according to claim 63, characterized in that the composition is a sun protection product for animals.

69. A betulin derivative of the general formula I′, or a pharmaceutically acceptable salt thereof, where in formula I′ 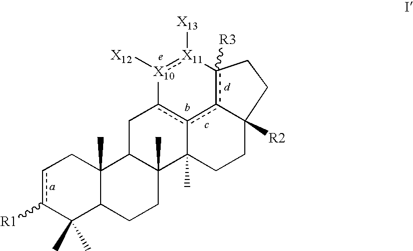
E. R1=OH;
F. R2=CH₂O(C═O)CH₂(CHR_{g})COOY where R_{g}═C₄-C₂₂ linear or branched alkyl or alkenyl group, Y═H, Na, K, Ca, Mg, C₁-C₄-alkyl group or NRₕ where Rₕ═H or C₁-C₁-alkyl group;
G. R3=CH₂═CCH₃; and
H. X₁₀═X₁₁═H, X₁₂═X₁₃=absent, a, b, c, and d each represent a single bond and e is absent; or
R1=OH;
R2=CH₂ORᵢ where Rᵢ=2,5-diaminopentanoyl, 2-(acetylamino)benzoyl, N,N,N-trimethyl-2-oxoethanaminium or isostearyl group;
R3=CH₂═CCH₃; and
X₁₀═X₁₁═H, X₁₂═X₁₃=absent, a, b, c, and d each represent a single bond and e is absent; or
R1=OH;
R2=CH₂ORₙ or CH₂O(C═O)CH₂OR′ where R′=verbenzyl, terpinyl, thymyl, carvacryl, menthyl, cinnamyl, curcuminyl, eugenyl, bornyl, isobornyl, longifolyl, isolongifolyl, globutyl, epiglobutyl, cedryl or epicedryl group and Rₙ=retinoyl group; and;
R3=CH₂—CCH₃; and
X₁₀═X₁₁═H, X₁₂═X₁₃=absent, a, b, c, and d each represent a single bond and e is absent; or
R1=(C═O)CH₂(CHR_{c})COOY where Rₓ═C₄-C₂₂ linear or branched alkyl or alkenyl group, Y Na, K, Ca, Mg, C₁-C₄ alkyl group or NRₕ, where Rₕ═H or a C₁-C₄ alkyl group;
R2=CH₂O(C═O)CH₂(CHR_{d})COOY where R_{d}═C₄-C₂₂ linear or branched alkyl or alkenyl group, Y═H, Na, K, Ca, Mg, C₁-C₄ alkyl group or NRₖ where Rₖ═H or a C₁-C₄ alkyl group;
R3=CH₂═CCH₃; and
X₁₀═X₁₁═H, X₁₂═X₁₃=absent, a, b, c, and d each represent a single bond and e is absent; or
R1=ORᵣ where Rr=2,5-diaminopentanoyl, 2-(acetylamino)benzoyl, N,N,N-trimethyl-2-oxoethanaminium or isostearoyl group;
R2=CH₂ORₚ where Rₚ=2,5-diaminopentanoyl, 2-(acetylamino)benzoyl, N,N,N-trimethyl-2-oxoethanaminium or isostearoyl group
R3=CH₂═CCH₃; and
X₁₀═X₁₁═H, X₁₂═X₁₃=absent, a, b, c, and d each represent a single bond and e is absent; or
R1=ORᵥ or O(C═O)CH₂OR′ where R′=verbenzyl, terpinyl, thymyl, carvacryl, menthyl, cinnamyl, curcuminyl, eugenyl, bornyl, isobornyl, longifolyl, isolongifolyl, globutyl, epiglobutyl, cedryl or epicedryl group and retinoyl group
R2=CH₂ORᵤ or CH₂O(C═O)CH₂OR′ where R′=verbenzyl, terpinyl, thymyl, carvacryl, menthyl, cinnamyl, curcuminyl, eugenyl, bornyl, isobornyl, longifolyl, isolongifolyl, globutyl epiglobutyl, cedryl or epicedryl group and Rᵤ=retinoyl group;
R3=CH₂═CCH₃; and
X₁₀═X₁₁═H, X₁₂═X₁₃=absent, a, b, c, and d each represent a single bond and e is absent; or
R1=OH;
R2=(C═O)NHCHRₓCOOY where Y═H, Na, K, Ca, Mg, C₁-C₄ alkyl group or NR_{y} where R_{y}═H or a C₁-C₄ alkyl group and Rₓ═CH₂CH₂CH₂CH₂NH₂ or 4-imidazolylmethyl group;
R3=CH₂═CCH₃; and
X₁₀═X₁₁═H, X₁₂═X₁₃=absent, a, b, c, and d each represent a single bond and e is absent; or
R1=oxo group;
R2=(C═O)NHCHRₓCOOY where Y═H, Na, K, Ca, Mg, C₁-C₄ alkyl group or NR_{y} where R_{y}═H or a C₁-C₄ alkyl group and Rₓ═CH₂CH₂CH₂CH₂NH₂, 4-imidazolylmethyl, 3-indolylmethyl CH₂COOZ or CH₂CH₂COOZ group and Z═R_{y};
R3=CH₂═CCH₃; and
X₁₀═X₁₁═H, X₁₂═X₁₃=absent, a, b, c, and d each represent a single bond and e is absent; or
R1=oxo group;
R2 (C═O)OR_{w} where R_{w}=verbenzyl, terpinyl, thymyl, carvacryl, menthyl, cinnamyl, curcuminyl, eugenyl, bornyl, isobornyl group, longifolyl, isolongifolyl, globutyl, cedryl or epicedryl group;
R3=CH₂═CCH₃; and
X₁₀═X₁₁═H, X₁₂═X₁₃=absent, a, b, c, and d each represent a single bond and e is absent; or
R1=OH or O—(C═O)R_{b} where R_{b}═C₃-C₈ cyclic or heterocyclic residue, substituted or unsubstituted phenyl or benzyl residue, C₁-C₂₂ alkyl or alkenyl group;
R2=CH₂OH or CH₂O—(C═O)R_{f} where R_{f}═C₃-C₈ cyclic or heterocyclic residue, substituted or un substituted phenyl or benzyl residue, C₁-C₂₂ alkyl or alkenyl group;
R3=H₂C═CCH₂R_{q} or CH₃CCH₂R_{q} where R_{q}=3-dihydrofuran-2,5-dione, 3-pyrrolidine-2,5-dione or CH(COORₒ)H₂COOR_{z} where Rₒ═H, Na, K, Ca, Mg or a C₁-C₂₂ linear or branched alkyl or alkenyl group and R_{z}═H, Na, K, Ca, Mg or a C₁-C₂₂ linear or branched alkyl or alkenyl group; and X₁₀═X₁₁═H, X₁₂═X₁₃=absent, a, b, c, and d each represent a single bond and e is absent; or
R1=H, OR_{z}, O(C═O)R_{b}, NRₐR_{z}, CN, ═NORₐ, CHO, (C═O)OR_{z}, SR_{z}, ═O or ═S where R_{z}═H, C₁-C₆ linear or branched alkyl or alkenyl group or an aromatic group ZZ shown below and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ or R1 corresponds to the partial structure XX shown below;
R2=CH₂OR_{z}, CH₂O(C═O)R_{b}, (C═O)OR_{b}, CH₂NRₐR_{z}, CH₂CN, CN, CH═NORₐ, CH₂CHO, CH₂(C═O)OR_{z}, CH₂SR_{z}, CH═O or CH═S where R_{z}═H, C₁-C₆ linear or branched alkyl or alkenyl group or an aromatic group ZZ and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ or R2 corresponds to the partial structure YY shown below, with the proviso that R1 or R2 comprises the group XX;
R3=CH₂═C—CH₃ or CH₃—CH—CH₃;
X₁₀═X₁═H, X₁₂═X₁₃=absent, a, b, c, and d independently represent a single or a double bond and e is absent;
the partial structures XX and YY where YY═CH₂XX being selected from the group consisting of: 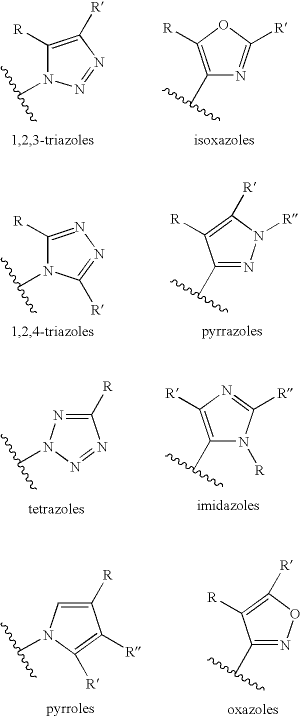
in which structures R, R′, and R″ independently represent H, an aromatic group ZZ, C₁-C₆ linear or branched alkyl or alkenyl group, the aromatic group ZZ being of the form: 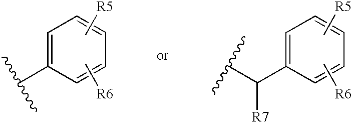
where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen, nitro, carboxy, carboxyl, acetyl, R5-R6 forms a cyclic methylenedioxy group, sulfate, cyano, hydroxy or trifluoromethyl group; or
R1=H, OR_{z}, NRₐR_{z}, CN, CHO, (C═O)OR_{z}, O(C═O)R_{b}, O(C═O)NHR_{f}, SR_{z}, ═O or ═S where R_{z}═H, C₁-C₆ linear or branched alkyl or alkenyl group or an aromatic group ZZ and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ or R_{b} corresponds to the partial structure YX shown below and R_{f}H, C₁-C₆ linear or branched alkyl or alkenyl group or an aromatic group ZZ or R_{f} corresponds to the partial structure YX shown below;
R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{f}, CH₂SR_{z}, CH═O or CH═S where R_{z}═H, C₁-C₆ linear or branched alkyl or alkenyl group or an aromatic group ZZ and Rₐ═H, C₁-C₆ linear or branched alkyl or alkenyl group, or an aromatic group ZZ and R_{b}═H, C₁-C₂₂ linear or branched alkyl or alkenyl group or an aromatic group ZZ or R_{b} corresponds to the partial structure YX shown below and R_{f}═H, C₁-C₆ linear or branched alkyl or alkenyl group or an aromatic group ZZ or R_{f} corresponds to the partial structure YX shown below, with the proviso that R1 or R2 comprises the group YX;
R3=CH₂═C—CH₃ or CH₃—CH—CH₃;
X₁₀═X₁₁═H, X₁₂═X₁₃=absent, a, b, c, and d independently represent a single or a double bond; and e is absent, said aromatic group ZZ being of the form: 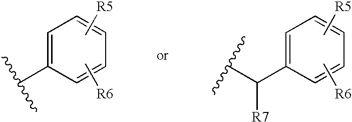
where R5, R6 and/or R7 may be H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen (fluoro, chloro, bromo, iodo), nitro, carboxy, carboxyl, acetyl, R5-R6 forms a cyclic methylenedioxy group, sulfate, cyano, hydroxy or trifluoromethyl group; and
the partial structure R_{f} or R_{b} is of the form YX: 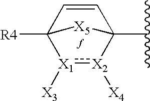
where R4=H or a C₁-C₂₀ linear or branched alkyl or alkenyl group or an aromatic group ZZ, X₅=absent, C, O, N or S, X₁-X₂ forms a cyclic partial structure of the form: X₁—(X₃═X₆)—X₇—(X₄═X₈)—X₂ where X₁═X₂═C or N, X₃═X₄═C, X₆═X₈═O, S or absent, X₇═C, O, S or N—X₉ where X₉═H, C₁-C₆ linear or branched alkyl or alkenyl group or an aromatic group ZZ, and f is a single or a double bond; or
R1=H, OR, NRₐR_{z}, CN, CHO, (C═O)OR_{z}, O(C═O)R_{b}, O(C═O)NHR_{z}, SR_{z}, ═O or ═S where R_{z}═H, C₁-C₆ linear or branched alkyl or alkenyl group or an aromatic group ZZ and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ;
R2=CH₂OR_{z}, (C═O)OR_{b}, CH₂NRₐR_{z}, CH₂CN, CH₂CHO, CH₂(C═O)OR_{z}, CH₂O(C═O)R_{b}, CH₂O(C═O)NHR_{z}, CH₂SR_{z}, CH═O or CH═S where R_{z}═H, C₁-C₆ linear or branched alkyl or alkenyl group or an aromatic group ZZ and Rₐ═H, C₁-C₆ linear or branched alkyl or alkenyl group or an aromatic group ZZ and R_{b}═H, C₁-C₂₂ linear or branched alkyl or alkenyl group or an aromatic group ZZ;
R3=CH₂═C—CH₃ or CH₃—CH—CH₃;
ZZ being of the form: 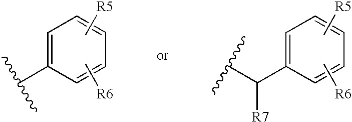
where R5, R6 and/or R7 is H, a C₁-C₆ linear or branched alkyl or alkenyl group, a C₁-C₆ linear or branched alkyl or alkenyl ether, R5-R6 forms a cyclic C₂-C₆ alkyl or alkenyl group, halogen, nitro, carboxy, carboxyl, acetyl, R5-R6 forms a cyclic methylenedioxy group, sulfate, cyano, hydroxy or trifluoromethyl, at X₁₀—X₁₁, a cyclic or heterocyclic partial structure having the form X₁₀—(X₁₂═X₁₄)—X₁₅—(X₁₃═X₁₆)—X₁₁ is present where X₁₀═X₁₁═C or N, X₁₂═X₁₃═C, X₁₄═X₁₆═O, S or absent, X₁₅═C, O, S or N—X₁₇ where X₁₇═H, a C₁-C₆ linear or branched alkyl or alkenyl group or an aromatic group ZZ, with the proviso that X₁₇ is not phenyl; and a, b, c, d and e independently represent double or single bonds; and
betulin 28-acetic acid methyl ester.

70. Betulin derivative according to claim 69, characterized in that the betulin derivative is selected from the group consisting of betulin 3,28-C₁₈-dialkenylsuccinic acid diester, betulin-28-yl (5-isopropyl-2-methylphenoxy)-acetate, 28-aspartate amide dimethylester of betulonic acid, betulin-28-yl 2-(acetylamino)benzoate, Diels-Alder adduct of 3β-28-diacetoxylupa-12,18-diene and urazole, Diels-Alder adduct of 3β-28-diacetoxylupa-12,18-diene and 4-methylurazole, Diels-Alder adduct of 3β-28-diacetoxylupa-12,18-diene and p-fluoro-4-phenylurazole, Diels-Alder adduct of 3β-28-diacetoxylupa-12,18-diene and m-methoxy-4-phenylurazole, Diels-Alder adduct of 3β-28-diacetoxylupa-12,18-diene and 1-naphthylurazole, and Diels-Alder adduct of 3β-28-diacetoxylupa-12,18-diene and 1,3-dioxol-5-ylurazole.

71. Cosmetic or pharmaceutical composition, characterized in that it comprises between 0.01 and 20% by weight of betulonic acid and between 0.01 and 20% by weight of one or more betulin derivative(s) according to claim 69.

72. Cosmetic or pharmaceutical composition according to claim 71, characterized in that it comprises between 0.1 and 10% by weight of betulonic acid.

73. Cosmetic or pharmaceutical composition according to claim 71, characterized in that it is a powder containing betulonic acid and one or more betulin derivative(s) powdered as such or in combination with one or more constituent(s) or excipient(s) selected from the group consisting of additives, fillers, carriers, vectors, surfactants, solvents, UV protection agents, antioxidants, preserving agents, colouring agents, alcohols, waxes, oils, fats, perfumes, thickeners, and pharmaceutically and/or cosmetically active agents.

74. Cosmetic or pharmaceutical composition according to claim 71, characterized in that it is a sun protection agent containing betulonic acid and one or more betulin derivative(s) formulated with one or more constituent(s) or excipient(s) selected from the group consisting of additives, fillers, carriers, vectors, surfactants, solvents, UV protection agents, antioxidants, preserving agents, colouring agents, alcohols, waxes, oils, fats, perfumes, thickeners, and pharmaceutically and/or cosmetically active agents.

75. Cosmetic or pharmaceutical composition according to claim 71, characterized in that it is a preparation for skin containing betulonic acid and one or more betulin derivative(s) formulated with one or more constituent(s) or excipient(s) selected from the group consisting of additives, fillers, carriers, vectors, surfactants, solvents, UV protection agents, antioxidants, preserving agents, colouring agents, alcohols, waxes, oils, fats, perfumes, thickeners, and pharmaceutically and/or cosmetically active agents.

76. Cosmetic or pharmaceutical composition according to claim 71, characterized in that it is a preparation for hair containing betulonic acid and one or more betulin derivative(s) formulated with one or more constituent(s) or excipient(s) selected from the group consisting of additives, fillers, carriers, vectors, surfactants, solvents, UV protection agents, antioxidants, preserving agents, colouring agents, alcohols, waxes, oils, fats, perfumes, thickeners, and pharmaceutically and/or cosmetically active agents.

77. Cosmetic or pharmaceutical composition according to claim 71, characterized in that it is a lipstick containing betulonic acid and one or more betulin derivative(s) formulated with one or more constituent(s) or excipient(s) selected from the group consisting of additives, fillers, carriers, vectors, surfactants, solvents, UV protection agents, antioxidants, preserving agents, colouring agents, alcohols, waxes, oils, fats, perfumes, thickeners, and pharmaceutically and/or cosmetically active agents.

78. Cosmetic or pharmaceutical composition according to claim 71, characterized in that it is a preparation for animals, containing betulonic acid and one or more betulin derivative(s) formulated with one or more constituent(s) or excipient(s) selected from the group consisting of additives, fillers, carriers, vectors, surfactants, solvents, UV protection agents, antioxidants, preserving agents, colouring agents, alcohols, waxes, oils, fats, perfumes, thickeners, and pharmaceutically and/or cosmetically active agents.
